(19) United States
(12) Patent Application Publication
(10) Pub. No.: US 2023/0338674 A1
(43) Pub. Date: Oct. 26, 2023

(54) OPEN SURGERY PATIENT INTERFACE

(71) Applicant: Fisher & Paykel Healthcare Limited, Auckland (NZ)

(72) Inventors: Jesus Antonio AMADOR NORIEGA, Auckland (NZ); Zane Paul GELL, Auckland (NZ); Eu-Lee TEH, Auckland (NZ); Bernard Tsz Lun IP, Auckland (NZ); Rory Alexander MONRO, Auckland (NZ); James Robert Jarmey GREENFIELD, Auckland (NZ); Donald Roy KURIGER, Auckland (NZ); Ali Ghalib Abdul Rahman GHALIB, Auckland (NZ); Jemma Tamsin SOMERVILLE, Auckland (NZ); Scott Hamish FREEBAIRN, Auckland (NZ); Zach Jonathan WARNER, Auckland (NZ); Benjamin Elliot Hardinge PEGMAN, Auckland (NZ)

(21) Appl. No.: 18/001,756

(22) PCT Filed: Jun. 17, 2021
(86) PCT No.: PCT/IB2021/055346

§ 371 Date: Dec. 14, 2022

Related U.S. Application Data
(60) Provisional application No. 63/040,376, filed on Jun. 17, 2020.
(60) Provisional application No. 63/061,694, filed on Aug. 5, 2020.

(51) Int. Cl. A61M 13/00

(52) U.S. Cl. CPC A61M 13/003; A61M 2206/11; A61M 2205/362

(57) ABSTRACT

A device for delivering gas into a surgical cavity comprising a patient interface having an inlet, fluidly connected with a gas source, and an outlet, the patient interface being configured to deliver a conditioned flow of gas into the surgical cavity. The conditioned flow of gas may have the properties of having a reduced outlet velocity relative to the velocity of the inlet gas, or having a diffuse flow, or being heated, or being substantially laminar or non-turbulent, or a combination thereof. The invention may also comprise an attachment mechanism to attach to or anchor the patient interface.

### TECHNICAL FIELD

The present disclosure relates to an open surgery medical gases delivery system, and more particularly a patient interface for use in an open surgery medical gases delivery system.

### BACKGROUND ART

Insufflation gases can be used in surgery for a variety of purposes. In open surgery, gas can be insufflated into a body cavity for de-airing, as in cardiac or thoracic surgery, for example. The insufflation gas can be selected from air or carbon dioxide (CO₂).

One of the biggest barriers that prevents the successful delivery of gas into a surgical wound or cavity is the integration of the gas delivery system into the surrounding surgical work field. This is primarily due to the large amount of variation that exists in surgical procedures, incision types and locations, patient demographics, surgeon preferences, wound preparation techniques, wound retraction systems and patient draping and warming systems.

In this specification, where reference has been made to external sources of information, including patent specifications and other documents, this is generally for the purpose of providing context for discussing the features of the present disclosure. Unless stated otherwise, reference to such sources of information is not to be construed, in any jurisdiction, as an admission that such sources of information are prior art or form part of the common general knowledge in the art.

### SUMMARY

Disclosed is a patient interface for protecting a surgical cavity, comprising:

one or more enclosing or outer layers defining at least in part a gas flow path configured or configurable to direct gases to the surgical site, the gas flow path having a first end and an opposed second end,
an inlet positioned at the first end, and
an outlet positioned at the second end.

Disclosed is a device, for delivering gas into a surgical cavity, comprising
a patient interface fluidly connected with a gas source, the patient interface having an outlet to deliver a conditioned flow of gas into the surgical cavity.

Disclosed is a device, for delivering gas into a surgical cavity, comprising
a patient interface having an inlet fluidly connected with a gas source and an outlet, the patient interface configured to reduce velocity of gas between the inlet and the outlet.

Disclosed is a device, for delivering gas into a surgical cavity, comprising
a patient interface having an inlet fluidly connected with a gas source and an outlet, the patient interface comprising a gas-permeable material and being configured to reduce the velocity of gas between the inlet and the outlet.

Disclosed is a device, for delivering gas into a surgical cavity, comprising
a patient interface fluidly connected with a gas source, the patient interface comprising a support structure, an inlet to the support structure fluidly connected with the outlet of the delivery assembly, and an outlet from the support structure to deliver a conditioned flow of gas into the surgical cavity.

Disclosed is a device, for delivering gas into a surgical cavity, comprising
a delivery assembly having an inlet fluidly connected with a gas source, and an outlet,
a patient interface comprising a support structure, an inlet to the support structure fluidly connected with the outlet of the delivery assembly, and an outlet from the support structure to deliver a conditioned flow of gas into the surgical cavity.

Disclosed is a device for delivering gas into a surgical cavity, the device comprising:
a patient interface having an inlet fluidly connectable with a gas source, and an outlet, and
wherein the patient interface is adapted to manipulate the flow of gas.

Disclosed is a device for delivering gas into a surgical cavity, the device comprising:
a patient interface having an inlet fluidly connectable with a gas source, and an outlet, and
wherein the patient interface comprises a gas permeable material configured to reduce velocity of gas flow between the inlet and the outlet.

Disclosed is a device, for delivering gas into a surgical cavity, comprising
a delivery assembly having an inlet fluidly connected with a gas source, and an outlet,
a patient interface comprising
   a support structure comprising a gas-permeable material,
   an inlet to the support structure fluidly connected with the delivery assembly, and
   an outlet from the support structure to deliver a conditioned flow of gas into the surgical cavity.

Disclosed is a device for delivering gas into a surgical cavity, the device comprising:
a patient interface having an inlet fluidly connectable with a gas source, and an outlet,
a mechanism to condition the flow of gas from the outlet, and
an attachment means.

Disclosed is a device, for delivering gas into a surgical cavity, comprising
a delivery assembly having an inlet and an outlet, the inlet in fluid communication with a gas source to provide gas flow in the delivery assembly,
an interface comprising
   a support structure having an inlet in fluid communication with the delivery assembly, and an outlet to deliver a conditioned flow of gas to a gas-permeable material that is adjacent the support structure outlet,
   one or more gas-impermeable layers that coats or layers at least a portion of the support structure and gas-permeable material, the one or more gas-impermeable layers adapted to maintain the gas-permeable material adjacent the outlet of the support structure,
   an interface outlet defined by the surface of the gas-permeable material not covered or layered in the one or more gas-impermeable layers, such that the flow of gas from the interface inlet to the interface outlet must pass through the gas-permeable material.

Disclosed is an interface, for delivering gas into a surgical cavity, comprising
a support structure having an inlet for gas flow, and an outlet to deliver a conditioned flow of gas to a gas-permeable material that is adjacent the support structure outlet,
one or more gas-impermeable layers that coats or layers at least a portion of the support structure and gas-permeable material, the one or more gas-impermeable layers adapted to maintain the gas-permeable material adjacent the outlet of the support structure,
an interface outlet defined by the surface of the gas-permeable material not covered or layered in the one or more gas-impermeable layers, such that the flow of gas from the interface inlet to the interface outlet must pass through the gas-permeable material.

Disclosed is a device, for delivering gas into a surgical cavity, comprising
a delivery assembly having an inlet and an outlet, the inlet in fluid communication with a gas source to provide gas flow in the delivery assembly,
an interface comprising
   a support structure having a volume, in which a portion of the volume comprises a gas-permeable material,
   an inlet to the support structure in fluid communication with the delivery assembly, and
   an outlet from the support structure to deliver a conditioned flow of gas into the surgical cavity, and
   wherein the gas-permeable material is:
      located at, or defines, the outlet to define an unfilled portion of support structure between the gas-permeable material and the support structure inlet, or located distally of the outlet to define an unfilled portion of support structure between the gas-permeable material and the support structure outlet,
such that the flow of gas from the interface inlet to the interface outlet must pass through the gas-permeable material.

Disclosed is a device, for delivering gas into an open surgical cavity, comprising
a delivery assembly having an inlet and an outlet, the inlet in fluid communication with a gas source,
a patient interface having an inlet in fluid communication with the outlet of the delivery assembly and comprising a diffuser,
the device configured to deliver gas via a patient interface outlet into the open surgical cavity, the diffuser having an outlet area of at least 180 mm².

Disclosed is a device, for delivering gas into a surgical cavity, comprising
a delivery assembly having an inlet and an outlet, the inlet in fluid communication with a gas source to provide gas flow of about 5 to about 20 L/min in the delivery assembly,
an interface comprising
   a support structure in which about 25 to about 75% by volume of the support structure comprises a gas-permeable material,
   an inlet to the support structure in fluid communication with the delivery assembly, and
   an outlet from the support structure to deliver a conditioned flow of gas into the surgical cavity, the gas being conditioned to have a velocity of flow at the outlet of less than about 2 m/s.

Disclosed is a device, for delivering gas into a surgical cavity, comprising
a delivery assembly having an inlet and an outlet, the inlet in fluid communication with a gas source to provide gas flow of less than about 20 L/min,
an interface comprising
   a support structure in which about 25% to about 75% by volume of the support structure comprises a gas-permeable material,
   an inlet to the support structure in fluid communication with the delivery assembly, and
   an outlet having an effective outlet area of at least 1 mm², the outlet venting a conditioned flow of gas from the support structure into the surgical cavity at an outlet velocity of less than about 2 ms⁻¹.

Disclosed is a device for delivering gas into a surgical cavity comprising
a patient interface having an inlet, fluidly connected with a gas source, and an outlet,
the patient interface being configured to deliver a conditioned flow of gas to the surgical site,
the conditioned flow of gas
   i) having a reduced outlet velocity relative to the velocity of the inlet gas, or
   i) having a diffuse flow, or
   iii) being heated, or
   iv) being substantially laminar or non-turbulent, or
   v) any combination of (i) to (iv).

Disclosed is a patient interface for protecting a surgical cavity, comprising an inlet and an outlet, one or more enclosing or outer layers at least partly defining a gas flow path between the inlet and the outlet, the gas flow path including a formable section, and at least one deformable element positioned within the formable section, the at least one deformable element configured to form at least one bending direction for the formable section.

Disclosed is a patient interface for protecting a surgical cavity, comprising
an interface configured to condition gas flow into a substantially non-turbulent or laminar flow, the interface being shape for placement onto a surface at or adjacent a surgical site or surgical cavity for providing gas flow that prevents the entrance of ambient air into the non-turbulent or laminar gas flow and/or into the surgical cavity, and
the interface comprises
   a chamber having an inlet to receive gas from a gas source and an outlet spaced from the inlet, the height and width of the outlet configured to at least in part define the height and width of the gas flow for shielding the surgical site or surgical cavity from ambient air, and
   porous media within the chamber at least proximate the outlet through with the gas received in the inlet is passed to provide substantially non-turbulent or laminar flow from the outlet.

Disclosed is a method of treatment of a surgical cavity comprising positioning an interface as described in or adjacent a surgical site.

Disclosed is a method of protecting a patient from a loss of moisture and/or loss of heat during surgery comprising positioning an interface as described on a surface in or adjacent the surgical site.

Disclosed is a method of protecting a patient from an infection of a surgical site comprising positioning an interface as described on a surface adjacent a surgical site such that the gas from the outlet of the interface forms a protective barrier about the surgical site.

Any one or more of the following embodiments or configurations may relate to any of the above disclosures.

In some configurations the enclosing or outer layer is flexible.

In some configurations the enclosing or outer layer is a film or membrane.

In some configurations the enclosing or outer layer is formed of a breathable material that allows the passage of water molecules through the material

In some configurations the enclosing or outer layer is permeable to water vapour.

In some configurations the enclosing or outer layer is formed at least in part of a material having qualities that resist puckering and/or damage from surgical instruments.

In some configurations the enclosing or outer layer is resiliently deformable.

In some configurations the enclosing or outer layer is formed from polyurethane.

In some configurations the interface further comprises a second layer that surrounds at least in part the enclosing or outer layer.

In some configurations the second layer
a) defines at least a portion of the gas flow path, or
b) is located about the enclosing wall or outer membrane, or
c) has a thickness greater than the thickness of the enclosing wall or outer membrane, or
d) has a flexibility that is less than the flexibility of the enclosing wall or outer membrane, or
e) has a puncture-resistant that is greater than the puncture-resistant of the enclosing wall or outer membrane, or
f) is impermeable to fluids, or
g) any combination of one or more of (a) to (f).

In some configurations the conditioned flow of gas comprises a diffused flow.

As used herein, the term “diffused” or “diffused flow” and its grammatical equivalents means that the gas flow has reduced velocity compared to the flow of gas in the delivery assembly.

In one configuration a diffused flow has a velocity reduced by about 10, 20, 30, 40, 50, 60, 70 or 80% relative to the velocity of the gas flow in the delivery assembly, and suitable ranges may be selected from between any of these values.

In one configuration a diffused flow has an expanded cross-sectional gas flow relative to the cross-sectional gas flow in the delivery assembly.

In one configuration the diffused flow has a gas flow expanded by at least 50, 60, 70, 80, 90, 100, 110, 120, 130, 140, or 150% relative to the gas flow in the delivery assembly

As used herein, the term diffuser and its grammatical equivalents means that a device or apparatus that delivers a diffused flow of gas.

In some configurations the interface comprises a delivery assembly fluidly connected between a gas source and the interface.

In some configurations the delivery assembly comprises a conditioning source.

In some configurations the conditioning source is a humidifier, a heater, or a humidifier and a heater.

In some configurations the conditioned flow of gas is humidified.

In some configurations the delivery assembly comprises a gas regulator, a flow meter, or a flow meter and a gas meter.

In some configurations the delivery assembly comprises one or more conduits adapted to connect to the gas supply or gas source and to connect to the interface.

In some configurations the interface comprises a filter between the gas source and the interface.

In some configurations the filter is located filter upstream of the conditioning source, downstream of the conditioning source, or forms part of the conditioning source.

In some configurations the device comprises an attachment mechanism for retaining the delivery assembly, the patient interface, or the delivery assembly and patient interface in a position within, or adjacent to the surgical cavity.

In some configurations the device has a gas flow between 0.1 to 20 Umin.

In some configurations the gas flow path exiting outlet is substantially in a single plane.

In some configurations the gases exiting the outlet are substantially laminar and/or non-turbulent.

In some configurations the gas flow path has substantially the same height or cross sectional area.

In some configurations the gases flow path has a constant width.

In some configurations the interface is configured such that the gas flow exiting the outlet has a velocity that is less than the gas flow velocity at the interface inlet.

In some configurations the device is configured to deliver gas via an interface outlet into the open surgical cavity at a supply pressure of less than 30 mmHg.

In some configurations the gas source is selected from bottled gases, a gas cylinder that provides pressurized gas, wall gases source, reticulated gases source, articulated gas source (e.g. pendant supply), ambient air or a flow generator or compressor that generates a gas flow.

In some configurations the gases source provides a continuous or intermittent flow of gases, that can be at a desired flow rate.

In some configurations the gas source can be any gas or gas mixture suitable for a particular surgical application, including therapeutic gases. Preferably the gas is selected from air, carbon dioxide (CO₂), nitrogen gas (N₂), nitrogen dioxide N₂O, Argon (Ar), Helium (He), or a mixture thereof.

In some configurations the density of the gas provided by the gas source is greater than air. Preferably the density of the gas provided by the gas source is greater than air where the outlet of the patient interface is at, adjacent, or above, the surgical cavity.

In some configurations the density of the gas provided by the gas source is the same, or similar, to air. Preferably the density of the gas provided by the gas source is the same, or similar, to air where the outlet of the patient interface is located within the surgical cavity.

In some configurations the velocity of gas flow in the delivery assembly is at least 2-fold higher than the outlet velocity of the patient interface.

In some configurations the device comprises a gas inlet tube that defines the gas inlet into the patient interface.

In some configurations the gas-permeable material is located distally to the outlet of the patient interface.

In some configurations the surface area of the outlet of the patient interface is about 6 to about 20% of the surface area of the gas-permeable material.

In some configurations the gas-permeable material is located at, or defines, the outlet.

In some configurations the delivery assembly fluidly connected between the gas source and the patient interface.

In some configurations the delivery assembly comprises a conditioning source.

In some configurations the conditioning source is a humidifier, a heater, or a humidifier and a heater.

In some configurations the delivery assembly comprises a gas regulator, a flow meter, or a flow meter and a gas meter.

In some configurations the patient interface comprises a support structure.

In some configurations the interface comprises a support structure at the first end, the support structure defining a housing inlet and housing outlet, the housing outlet being in fluid communication with the interface inlet and/or interface outlet.

In some configurations the support structure is a chamber.

In some configurations the chamber comprises a gas-permeable material.

In some configurations the support structure abuts the gas permeable substrate.

In some configurations the support structure encloses a portion of the gas permeable substrate.

In some configurations the height of the support structure is substantially the same height as the gas permeable substrate.

In some configurations the interface further comprises a tube having a first and second end, the first end connectable to the inlet of the support structure housing inlet.

In some configurations the tube first end connects to the housing inlet via a luer connection, a press fit connection, screw threat or friction fit connection.

In some configurations the tube second end has a luer lock connector.

In some configurations the tube is flexible.

In some configurations the gas permeable material is configured to reduce velocity of the gas flow.

In some configurations the gas-permeable material is: located at, or defines, the outlet of the patient interface to define a cavity in the support structure between the gas-permeable material and the inlet to the support structure inlet, or located distally to the outlet of the patient interface to define a cavity in the support structure between the gas-permeable material and the support structure outlet.

In some configurations about 25 to about 75% by volume of the support structure comprises a gas-permeable material.

In some configurations the gas permeable substrate has a front and rear face. An end of each of the front and rear face of the gas permeable substrate can abut the support structure.

In some configurations the support structure is a chamber and the gas permeable substrate fills at least a portion of the chamber. The gas permeable substrate may be contained within the chamber or at least a portion may extend outwardly of the chamber at or adjacent the outlet.

In some configurations the patient interface includes one or more adhesive layers.

In some configurations the interface defines a front surface and a rear surface.

In some configurations the rear surface further comprises an adhesive.

In some configurations the patient interface includes an adhesive layer on at least a portion of the rear surface of the patient interface, the front surface of the patient interface, or both the front and rear surface of the patient interface.

In some configurations the patient interface includes an adhesive layer on at least a portion of the front surface of the patient interface device.

In some configurations the adhesive is an adhesive layer.

In some configurations the adhesive layer comprises a removable backing that once removed exposes the adhesive layer.

In some configurations the interface comprises a removal tab.

In some configurations the removal tab is provided by the enclosing wall or outer membrane, or the second layer.

In some configurations the enclosing or outer layer includes a cut-out on the rear surface adjacent the interface outlet.

In some configurations at least a portion of a side region of the gas permeable material is absent any covering, as might otherwise be provided by the enclosing wall or outer membrane, the second layer, or the adhesive layer.

In some configurations the interface outlet is substantially rectangular in cross section.

In some configurations the edge of interface outlet is rounded.

In some configurations the patient interface includes a layer of material intended to protect the interface.

In some configurations the layer of material intended to protect the interface is provided by a separate protective layer of material.

In some configurations the layer of material intended to protect the interface is provided by a thickening of a layer of the interface.

In some configurations the support structure or chamber is formed of a rigid material such as plastic. The chamber may be at least partly formed of a flexible material such as silicon.

In some configurations the gas permeable substrate is enclosed by the wall or outer membrane.

In some configurations a portion at least of the surface area of the gas permeable substrate is coated or layered in one or more gas-impermeable layers.

In some configurations a gas-impermeable layer is a gas-impermeable film.

In some configurations a portion of the gas permeable substrate not coated or layered in a gas-impermeable layer defines the interface outlet.

In some configurations the gas permeable substrate is, or comprises, an open cell foam.

In some configurations the gas permeable substrate is flat.

In some configurations the gas permeable substrate is rectangular.

In some configurations the length and/or width of the gas permeable substrate is greater than height.

In some configurations the length of the gas permeable substrate is greater than the width.

In some configurations the width of the gas permeable substrate is greater than the length.

In some configurations the patient interface is configured to locate at least partly in the surgical cavity with at least part of the rear face of the patient interface adhered to a surface in or adjacent the surgical cavity, such as a surface of a surgical retractor.

In some configurations the patient interface is configured to locate with the outlet arranged at or adjacent the wound edge of the surgical cavity.

In some configurations at least a portion of the patient interface is flexible and/or deformable to allow the patient interface to be bent and to retain its shape.

In some configurations the patient interface comprises a deformable element to allow the patient interface to be bent and to retain its shape.

In some configurations the deformable element attaches to the support structure.

In some configurations the deformable element is malleable.

In some configurations the deformable element is formed from stainless steel or plastic.

In some configurations the deformable element is resiliently deformable.

In some configurations the deformable element is located within the gas permeable substrate, between an enclosing or outer layer and the gas permeable substrate, within an enclosing or outer layer, or outside of an enclosing or outer layers.

In some configurations the deformable element is shaped to conform substantially to the shape of a perimeter of the gas permeable substrate.

In some configurations the patient interface is bendable to substantially correspond with a configuration of the wound edge, and to retain its shape once bent into that configuration.

In some configurations the gas permeable substrate is removable from the support structure or chamber.

In some configurations the gas permeable substrate reduces the velocity of gases at the outlet relative to the inlet.

In some configurations the velocity of gas flow at the outlet of the patient interface is less than about 2 m/s.

In some configurations the velocity of gas flow at the outlet of the patient interface is less than about 0.8 ms⁻¹.

In some configurations the patient interface has an outlet having an effective outlet area of at least 1 mm², the outlet venting a conditioned flow of gas at an outlet velocity of less than about 2 ms⁻¹.

In some configurations the gas source provides a gas flow of about 5 to about 20 L/min.

In some configurations the patient interface has an effective outlet area of at least 180 mm².

In some configurations the pressure of the gas flow at the outlet of the patient interface is less than 30 mmHg.

In some configurations the delivery assembly comprises one or more conduits adapted to connect to the gas supply or gas source and to connect to the patient interface.

In some configurations the device comprises a retractor, to maintain the surgical cavity in an accessible condition, wherein the retractor is
separate to the patient interface, or
is part of the patient interface.

In some configurations the delivery assembly comprises a conditioning source to condition the gases prior to delivery to the surgical cavity. In some configurations, the conditioning source is a humidifier to humidify the gases delivered to the surgical cavity.

In some configurations the delivery assembly comprises a gas control unit, wherein the gas control unit controls the supply of gas.

In some configurations the device comprises a heating source, for heating the gas flow.

In some configurations the device comprises a gas control unit inlet and outlet, a humidifier inlet and outlet, and a patient interface inlet and outlet, the gas control unit in fluid communication with a gas source, the gas control outlet in fluid communication with the humidifier inlet, and the humidifier outlet in fluid communication with the interface inlet.

In some configurations the cross-sectional size of the patient interface expands between the patient interface inlet and patient interface outlet.

In some configurations the patient interface outlet is expanded relative to the patient interface inlet.

In some configurations the cross-sectional size of the total outlet area of the patient interface is 10, 15, 20, 25 or 30 times that of the cross-sectional size of the patient interface inlet.

In some configurations the patient interface comprises a smooth transition between the patient interface inlet and outlet.

In some configurations the flow rate of the gas flow through the delivery assembly is less than 10 L/min.

In some configurations the interface outlet surface area is about 6 to about 20% of the surface area of the gas-permeable material.

In some configurations the device is adapted to deliver gas at a flow rate of up to 20 L/min.

In some configurations the patient interface is adapted to vent gas at a velocity of less than 2 ms⁻¹.

In some configurations the patient interface is adapted to function as a retractor to maintain the surgical cavity in an open condition.

In some configurations the retractor and the patient interface are integrated.

In some configurations the patient interface is an extendable and lockable scaffold and comprises one or more outlets, the scaffold adapted for radial extension and retraction within the surgical cavity.

In some configurations the patient interface comprises an inflatable member, and wherein at least a portion of the inflatable member is located at, or about, the surgical cavity and comprises one or more outlets.

In some configurations the patient interface is an inflatable tube comprising one or more outlets and is adapted for inflation to apply radial force to the walls of the surgical cavity.

In some configurations the outlet(s) are positioned on the internal edge of the inflatable tube.

In some configurations the patient interface forms part of a surgical drape that at least partly surrounds the surgical cavity, the surgical drape having one or more gas pathways and one or more outlets adjacent the surgical cavity.

In some configurations the patient interface is L-shaped.

In some configurations the patient interface is U-shaped.

In some configurations the patient interface outlet on a lower surface of the patient interface, to direct air downwards into the surgical cavity.

In some configurations the patient interface outlet on a vertical surface of the patient interface, to direct air horizontally.

In some configurations the patient interface is a flexible pad comprising a gas outlet zone having one or more gas flow paths for delivering air into the surgical cavity.

In some configurations the flexible pad includes an adhesive zone, preferably in the form of a film having a front gas venting surface and a rear adhesive surface.

In some configurations the pad is formed at least in part by a material that retains its shape when subjected to plastic deformation.

In some configurations the flexible pad includes an adhesive zone.

In some configurations the form of a flexible pad comprising a gas outlet surface an adhesive surface on an opposed side of the pad to the outlet.

In some configurations the pad is formed at least in part by a material that may be manipulated into a desired shape and retain to that desired shape

In some configurations the patient interface comprises one or more elongate portion(s) having a substantially tubular profile.

In some configurations a plurality of secondary elongate portions that stem off from a primary elongate portion.

In some configurations the elongate portion(s) (have one or more articulations. In one embodiment the primary elongate portion(s) has one or more articulations.

In some configurations the elongate portion(s) are made from material that may be manipulated into a desired shape and retain to that desired shape.

In some configurations at least one of the elongate portions are adapted to locate above the surgical cavity and wherein the outlet is directed downwards to the surgical cavity.

In some configurations the patient interface comprises a projection adapted to penetrate the tissue of patient.

In some configurations the projection is a hook.

In some configurations the patient interface includes a zone of greater weight and/or density.

In some configurations the zone of greater weight and/or density is located distal to the patient interface inlet.

In some configurations the patient interface comprises a grip handle and a shaft, the outlet of the patient interface located on the shaft distally from the handle.

In some configurations the patient interface comprises one or more arms that each comprises one or more interface outlets or a plurality of outlets for the patient interface.

In some configurations the patient interface is substantially “Y”-shaped.

In some configurations the patient interface comprises at least two separate arms.

In some configurations the patient interface comprises at least one outlet on the inner surface of each arm of the patient interface.

In some configurations the patient interface comprises one or more adjustable loop(s).

In some configurations the patient interface comprises a slidable collar that forms a loop.

In some configurations the patient interface has one or more gas colliding surfaces to which the gas collides prior to exiting the patient interface at the patient interface outlet.

In some configurations the patient interface comprises two or more interference gas flow paths between the patient interface inlet and patient interface outlet.

In some configurations the patient interface comprises two or more gas outlets, and wherein the outlets are directed substantially at each other.

In some configurations the outlets are positioned on opposed sides of the surgical cavity relative to each other.

In some configurations the patient interface is suspended over the surgical cavity, and wherein either the patient interface or the delivery assembly is positionable above the surgical cavity by an attachment mechanism.

In some configurations a portion of the patient interface, or the delivery assembly, is wound about a reel.

In some configurations the patient interface is held in position by a moveable elongate stand.

In some configurations the patient interface comprises a vent, and wherein the vent is located above the surgical cavity.

In some configurations the vent is located in the ceiling above the surgical cavity.

In some configurations the patient interface integrates to, or connects into, an existing air conditioning or ventilation system.

In some configurations the patient interface comprises a plurality of outlets.

In some configurations the outlets are distributed evenly along a length of the patient interface.

In some configurations the outlets are distributed irregularly along a length of the patient interface.

In some configurations the outlets are holes in the surface of the patient interface, and the holes are of uniform size.

In some configurations the outlets are holes in the surface of the patient interface, and the holes vary in size.

In some configurations the patient interface or the patient interface outlet comprises a porous medium, the porous medium having a plurality of gas flow paths and plurality of outlets.

In some configurations the porous medium is a soft porous medium selected from weaved fabric, felt, porous films, woven mesh, fibrous materials.

In some configurations the porous medium is a hard and/or rigid porous medium selected from sintered metal, sintered polymer, sintered plastic or sintered ceramic.

In some configurations the porous medium is a granular substrate selected from sand, carbon, garnet or anthracite.

In some configurations the patient interface comprises a hinged or articulated portion, wherein the patient interface outlet is located on the hinged or articulated portion.

In some configurations the hinged or articulated portion is connected to a first portion of the patient interface via a pair of pins and corresponding recesses.

In some configurations the hinged or articulated portion is connected to a first portion of the patient interface via a cylinder located at or adjacent the interface between the first portion and the hinged or articulated portion.

In some configurations the cylinder has a plurality of apertures.

In some configurations the retractor is formed of a malleable material and comprises one or more attachment mechanisms for the delivery assembly, patient interface, or delivery assembly and patient interface.

In some configurations the retractor includes a heating source to heat the gas.

In some configurations the retractor comprises one or more gas flow paths between the gas source and the patient interface outlet.

In some configurations the retractor comprises a replaceable component, and wherein the replaceable component comprises the gas flow path.

In some configurations the retractor comprises at least two arms to retract the surgical cavity, and wherein the two arms are located on opposed sides of the surgical cavity.

In some configurations at least a pair of arms of the retractor (each member located on opposed sides of the surgical cavity), comprise a gas flow path and a diffusion outlet.

In some configurations an attachment mechanism that attaches the patient interface and/or delivery assembly to any one or more of a surgical drape, the patient, a retractor, a surgical tool or furniture.

In some configurations the attachment mechanism incorporates a two-part clip or bracket that comprises a base and a bracket interposed over the base, wherein the clip comprises an aperture between the clip and bracket.

In some configurations the clip additionally comprises a slot.

In some configurations the two-part clip retains the retractor, the patient interface, or the retractor and patient interface.

In some configurations the retractor is held within the aperture.

In some configurations the bracket connects to the base with magnetic interactors.

In some configurations fasteners connect the bracket to the base.

In some configurations the base comprises a rotating joint.

In some configurations the clip is in the form of a ring clip element, comprising a ring with a central aperture and a pair of fastening arms.

In some configurations the clamping surface of the ring clip element comprises foam, rubber, silicone or a combination thereof.

In some configurations the ring clip element is fastened or clipped to the patient's skin, a surgical drape, or surgical equipment.

In some configurations the ring clip element is formed integrally with the delivery assembly.

In some configurations the attachment mechanism comprises a suction cup for attachment to a surgical drape, the patient, a surgical tool or furniture.

In some configurations the attachment mechanism comprises an adhesive pad or strip for attachment to a surgical drape, the patient, a surgical tool or furniture.

In some configurations the adhesive pad or strip comprises adhesive on both the top and lower surfaces of the adhesive pad or strip.

In some configurations the adhesive pad or strip is attached to a surface within the surgical site.

In some configurations the attachment mechanism attaches to the delivery assembly, patient interface, or delivery assembly and patient interface to the retractor.

In some configurations the attachment mechanism is a clip, a tie, a band, a releasable mechanical fastener such as a hook and loop structure (e.g. Velcro® strap), an elastic strap, bent wire or knotted string.

In some configurations the tie is a resilient adhesive tie.

In some configurations the attachment mechanism comprises a releasable mechanical fastener (e.g. Velcro® strap), having an adhesive surface for attachment to a surgical drape, the patient, a surgical tool or furniture, and a corresponding Velcro® sleeve about the delivery assembly, the patient interface or the delivery assembly and patient interface.

In some configurations the attachment mechanism is a bracket that covers the delivery assembly, the patient interface, or the delivery assembly and patient interface, the bracket attaching to the patient's skin, the surgical drape or surgical instrument or furniture.

In some configurations the cover is a sleeve.

In some configurations the bracket is secured to the patient's skin with sutures, glue or adhesive.

In some configurations the attachment mechanism comprises a magnetic pad, magnetic strip or pad for positioning on, or about, a surgical drape, the patient, a surgical tool or furniture, and a corresponding mating element about the delivery assembly, the patient interface or both the delivery assembly and the patient interface.

In some configurations the magnetic pad includes an adhesive surface.

In some configurations a surface of the patient interface is substantially planar and comprises glue for attachment to a surface of the surgical cavity, a surgical drape, a surgical tool or furniture.

In some configurations a cover that extends over the delivery assembly, the patient interface, or both the delivery assembly and the patient interface, the cover being attachable to a surgical drape, or patient surface by a suitable fastener.

In some configurations the cover is formed of a rigid material, a resilient material, or a soft and pliable material.

In some configurations the cover is moulded to form about or over the wound edge.

In some configurations the attachment mechanism is a clamp for attachment about the wall of the surgical cavity, the clamp comprising an attachment for the delivery assembly, the patient interface or the delivery assembly and patient interface.

In some configurations the device comprises two complementary pieces that combine, with one piece having an aperture or slot, and the other piece a complementary projection.

In some configurations the clamp is height adjustable.

In some configurations the height adjustable mechanism is a ratchet mechanism.

In some configurations the clamp is a C-clamp or a G-clamp.

In some configurations the attachment mechanism comprises
an elongate rail attachable to, or adjacent, the surgical cavity, and
a clip for attachment to the rail, the clip being moveable along said rail and the clip providing for attachment of the delivery assembly, the patient interface or the delivery assembly and patient interface.

In some configurations the elongate rail is integrated into a retractor.

In some configurations the rail may comprise two vertically spaced tracks for attachment of the delivery assembly, the patient interface, or both the delivery assembly and the patient interface.

In some configurations the rail comprises an “L”-shape or is in the form of a ring.

In some configurations the attachment mechanism is attached to a surgical drape, and wherein the drape covers at least the edge of the surgical cavity, and optionally at least a portion of the wall of the surgical cavity.

In some configurations the attachment mechanism is attached to a frame that extends the perimeter of the edge of the surgical cavity.

In some configurations the rail is a circular railing system provided in a ring configuration located in the surgical site such that it surrounds the surgical cavity.

In some configurations the attachment mechanism comprises one or more projections for passage through a surgical drape, each projection including a screw thread.

In some configurations the delivery assembly, the patient interface or the delivery assembly and patient interface comprise a plurality of projections less than 5 mm in length, the projections formed from a soft and/or flexible material.

In some configurations one or more zones of the delivery assembly, the patient interface or the delivery assembly and patient interface are covered in the projections, and wherein the projections lead to an at least 5-fold increase in the surface area of the zones.

In some configurations the attachment mechanism is one or more weighted anchors located on the patient interface, or on the delivery assembly adjacent the patient interface, and wherein the patient interface or delivery assembly are adapted to position the one or more weighted anchor in or adjacent the surgical cavity when in use.

In some configurations the attachment mechanism comprises a mechanical claw having two or more fingers, the mechanical claw adapted to include a release mechanism.

In some configurations the mechanical claw includes a mechanically or pneumatically operated release mechanism.

In some configurations the delivery assembly, patient interface, or delivery assembly and patient interface comprises an inflatable sleeve, the inflatable sleeve adapted for positioning inside the surgical cavity.

In some configurations the patient interface is suspended over the surgical cavity, and wherein the delivery assembly, patient interface, or delivery assembly and patient interface is positionable by at least two wires attached to opposed sides of the surgical cavity.

In some configurations the delivery assembly, patient interface, or delivery assembly and patient interface comprises a flexible region, wherein the flexible region wraps about a retractor, surgical instrument or furniture.

In some configurations the flexible region comprises corrugations.

In some configurations the delivery assembly, patient interface, or delivery assembly and patient interface is adapted for attachment to the limb of a medical practitioner.

In some configurations the patient interface delivers gas into the surgical cavity via insertion of the patient interface through an incision that creates a passage from the surgical site adjacent the surgical cavity to a patient wall of the surgical cavity.

In some configurations the patient interface comprises a plug, and wherein the plug is adapted for placement inside an incision located in the surgical cavity.

In some configurations the patient interface comprises a compressible component at or adjacent the patient interface outlet that presses into the surgical cavity.

In some configurations the compressible component is, or comprises, the patient interface outlet.

In some configurations the interface is located on a surface within the surgical cavity.

In some configurations the interface is located on or adjacent a wall of the surgical cavity.

In some configurations a gas source is activated to provide a gas flow to the gas flow path.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are considered to be expressly stated in this application in a similar manner.

It should be understood that alternative embodiments or configurations may comprise any or all combinations of two or more of the parts, elements or features illustrated, described or referred to in this specification.

Some embodiments of this disclosure may also be said broadly to consist or comprised in the parts, elements and features referred to or indicated in the specification of the application, individually or collectively, and any or all combinations of any two or more said parts, elements or features, and where specific integers are mentioned herein which have known equivalents in the art to which this disclosure relates, such known equivalents are deemed to be incorporated herein as if individually set forth.

The term “enclosing or outer layer”, and its grammatical variants, includes within its definition a film, barrier, wall or membrane and thus defines a layer that is configured to define at least a portion of the gas flow path of the interface.

The term “comprising” as used in this specification means ‘including’. When interpreting each statement in this specification that includes the term ‘comprising’, features other than that or those prefaced by the term may also be present. Related terms such as ‘comprise’ and ‘comprises’ are to be interpreted in the same manner.

As used herein the term ‘(s)’ following a noun means the plural and/or singular form of that noun.

As used herein the term ‘and/or’ means ‘and’ or ‘or’, or where the context allows both.

The disclosure discloses the foregoing and envisages constructions of which the following gives examples only.

### BRIEF DESCRIPTION OF THE DRAWINGS

Specific embodiments and modifications thereof will become apparent to those skilled in the art from the detailed description herein having reference to the Figures that follow, of which:

FIG. 1A shows a medical gases delivery system as described.

FIG. 1B shows an alternate medical gases delivery system as described.

FIG. 2A shows gradual flowing/flooding of gas over the upper edge of the surgical cavity.

FIG. 2B shows filling of a surgical cavity with gas with a small amount of jetting.

FIG. 2C shows filling of a surgical cavity with gas with jetting.

FIG. 3A shows a patient interface placed inside a surgical cavity.

FIG. 3B shows a patient interface placed adjacent to a surgical cavity.

FIG. 3C shows filling of a surgical cavity with top-down filing.

FIG. 3D shows filling of a surgical cavity with bottom-up filling.

FIGS. 4A to 4I show patient interfaces that comprise a support structure, which in FIGS. 4A and 4C-4H, the support structure comprises a chamber.

FIG. 4J shows one embodiment of a cross section through Z-Z of FIG. 4I.

FIG. 4K shows one embodiment of a cross section through Z-Z of FIG. 4I.

FIG. 4L shows an exploded view of a patient interface.

FIG. 4M shows a longitudinal cross section through the patient interface of FIG. 4L.

FIG. 5A shows a patient interface in the form of a gas-permeable substrate.

FIG. 5B demonstrates the flow of gas through the gas-permeable substrate.

FIGS. 6A and 6B shows a patient interface having a cross sectional area that expands along its length or from an inlet to an outlet.

FIG. 7A shows a patient interface with gas colliding flows.

FIGS. 7B and 7C shows the results of two gas colliding flows.

FIG. 7D shows a patient interface with internal baffles.

FIG. 8A shows a patient interface that is substantially U-shaped.

FIGS. 8B to 8D show a patient interface that is substantially L-shaped.

FIGS. 9A and 9B show a patient interface have a hinged or articulated portion.

FIGS. 10A and 10B show a patient interface having a hinged portion positioned close to the wound edge or upper patient wall of the surgical cavity.

FIG. 11 shows a patient interface in the form of a flexible pad.

FIG. 12A shows a patient interface in the form of a flexible pad with a highly flexible porous film.

FIG. 12B shows a patient interface of FIG. 12A located within the surgical cavity.

FIG. 13A shows a patient interface comprising one or more elongate body(s) having a substantially tubular profile.

FIG. 13B shows a patient interface of FIG. 13A with primary and secondary elongate bodies.

FIG. 14 shows a patient interface of FIG. 13A in the form of a loop.

FIG. 15 shows a patient interface that has one or more articulations.

FIGS. 16A and 16B shows a patient interface in the form of a flexible pad.

FIG. 17A shows a patient interface having one or more projections.

FIG. 17B shows a patient interface of FIG. 17A in position at the wound edge.

FIG. 18 shows a patient interface in the form of a surgical tool.

FIG. 19 shows a patient interface with arms in a bifurcated configuration.

FIG. 20 shows a patient interface comprising a slidable collar that forms a loop.

FIG. 21 shows a patient interface that locates above the surgical cavity.

FIG. 22 shows a patient interface that is suspended over the surgical cavity.

FIG. 23 shows a patient interface suspended above the surgical cavity.

FIG. 24 shows a patient interface integrated into, or forming part of, a vent positioned above the surgical site.

FIGS. 25A and 25B show a top perspective view of a patient interface formed as part of a surgical drape.

FIG. 25C shows a cross sectional view of the patient interface of FIGS. 25A and 25B.

FIG. 26 shows a patient interface that includes a surgical retractor or surgical retraction system.

FIGS. 27A to 27C show a retractor in the form of an expandable framework or linkage system.

FIG. 28 shows a retractor in the form of an inflatable conduit.

FIG. 29 shows an attachment system in the form of a malleable hook.

FIGS. 30A and 30B show an attachment system in the form of a two-part clip or bracket.

FIG. 31 shows an attachment system in the form of a ring clip.

FIGS. 32 and 33 shows an attachment system in the form of a clip.

FIGS. 34A and 34B shows an attachment system in the form of a clip fastener.

FIG. 35 shows an attachment system in the form of a wrap tie.

FIG. 36 shows an attachment system in the form of a resilient adhesive tie.

FIG. 37 shows an attachment system in the form of an attachment strip comprising complementary releasable mating structures.

FIGS. 38A and 38B show an attachment system that uses a magnetic element.

FIGS. 39A and 39B show an attachment system in the form of an adhesive pad or strip.

FIG. 40 shows an attachment system that uses that uses glue or other suitable surgical or medical adhesive.

FIG. 41 shows an attachment system that comprises a cover.

FIGS. 42 and 43A to 43C show an attachment system in the form of a clamp.

FIG. 44 shows an attachment system in the form of an elongate rail.

FIG. 45 shows an attachment system in the form of a rail with a ring configuration.

FIG. 46 shows an attachment system for attachment to a surgical drape.

FIGS. 47A and 47B show an attachment system comprising one or more projections for passage through a surgical drape.

FIG. 48 shows an attachment system comprising a plurality of projections.

FIG. 49A shows an attachment system comprising one or more weighted anchors.

FIG. 49B shows an enlargement of the attachment system of FIG. 49A.

FIG. 50 shows an attachment system in the form of a mechanical claw.

FIG. 51 shows an attachment system in the form of an inflatable sleeve.

FIG. 52 shows a pump for an inflatable sleeve of FIG. 51.

FIG. 53 shows an attachment system in the form of a flexible wrap.

FIG. 54 shows an attachment system for attachment to a limb of the surgeon or a surgical tool.

FIG. 55 shows an attachment system based on a side incision to the surgical cavity.

FIGS. 56A and 56B show an attachment system that includes a compressible component.

FIGS. 57A and 57B show a cross section through an interface having a plurality of slits/grooves.

FIGS. 57C to 57H show an interface having a plurality of slits/grooves.

FIG. 58 show an attachment mechanism, such as for attachment to a retractor.

### DETAILED DESCRIPTION

The present invention relates to open surgery medical gases delivery systems and to patient interfaces used in medical gases delivery systems.

Insufflation gases can be used in a variety of surgical procedures.

Surgical procedures of relevance are those that result in the creation of a surgical cavity. The surgical site is defined as the entirety of the operating space, including in and out of the surgical cavity. The surgical cavity is defined as the internal volume of the wound after incision, and is the volume intended to be filled with the gas. The wound edge is defined as the edges created by the incision which are then retracted back after the initial surgical incision. The upper patient wall is defined as the top layers of skin, fat, tissue that the surgeon cuts through to create an opening to create the surgical cavity.

In open surgical procedures, the patient's tissues are exposed to the atmospheric conditions of the operating theatre. This can result in cellular desiccation, evaporative cooling and airborne particles entering the wound. The delivery of heated and/or humidified gas has the potential to create a protective space while the surgeon is working within the wound that protects the patient's wound from the operating theatre environment. In open surgery, gases can be used in a body cavity, which can mitigate various risks associated with surgery, such as, but not limited to, decreasing the risk of air embolism or, decreasing the risk of infection. In open surgical applications, the insufflation system provides a generally constant flow of insufflation gas as a desired flow rate over an appropriate time period, which can encompass a portion or the entirety of the surgical procedure.

One or more components of the open surgery medical gases delivery systems may be biocompatible and/or sterilisable. The components that are sterilisable may be sterilizable by suitable means, such as sterilisation by heating and/or radiation.

The term “gases” is used herein broadly to refer to any gas and/or combination of gases that may be used in surgical applications, such as, carbon dioxide, helium, air, carbon dioxide combined with nitrous, carbon dioxide combined with oxygen, among others.

### 1. Overview

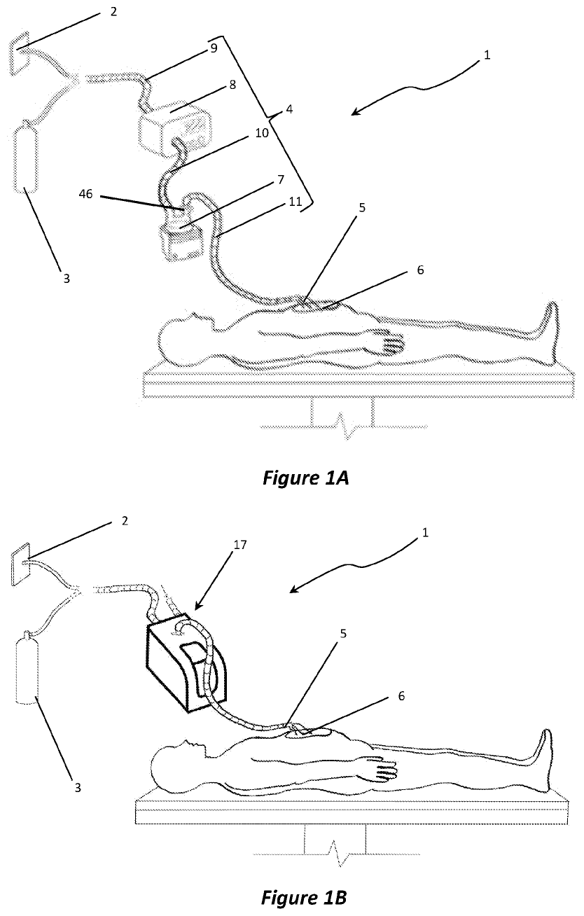

Shown in FIGS. 1A and 1B is an example medical gases delivery system 1 for open surgery. The medical gases delivery system 1 generally comprises a gas supply or gas source 2, 3, a delivery assembly 4 that has an inlet receiving gas from the gas supply or gas source 2, 3 and an outlet that connects to a patient interface 5 that delivers the gas into or adjacent the surgical cavity 6. The delivery assembly 4 may further comprise a gas control unit 8 and/or a gas conditioning source 7. The delivery assembly 4 may also comprise one or more filters 46. The one or more filters 46 can be located upstream and/or downstream of the gas conditioning source 7 and/or may be integral with the gas conditioning source 7 as shown in FIG. 1A. The one or more filters 46 can filter gas flow such that gas exiting the delivery assembly 4 is substantially sterile. The one or more filters 46 can comprise a high efficiency particulate air filter (HEPA), typically capable of removing at least 90% of airborne particles 0.3 micrometers and larger in diameter.

The gas supply or gas source 2, 3 can be from a range of sources and types. The gas supply or gas source 2, 3 can be any suitable supply source such as bottled gases, cylinder 3 that provides pressurized gas, wall gases source, reticulated gases source, articulated gas source (e.g. pendant supply), ambient air or a flow generator or compressor that generates a gas flow, for example, from ambient air. The gases supply or gases source 2, 3 can provide a continuous or intermittent flow of gases, that can be delivered at a desired flow rate. For example, FIGS. 1a and 1b shows two examples of a gas source. FIG. 1a shows that the gas source can be provided either by a bottled gas or cylinder 3, or a wall source 2 and FIG. 1b shows that the gas source is provided primarily from ambient air via a flow generator.

The gas supply or gas source 2, 3 can provide one or more insufflation gases. The insufflation gas can be any gas or gas mixture suitable for a particular surgical application, including therapeutic gases, which may be selected from air or carbon dioxide (CO₂) or a mixture thereof. Other insufflation gases that may be used, either singly or in combination with other gases, include nitrogen gas (N₂), nitrogen dioxide N₂O, Argon (Ar) and Helium (He).

In the medical gases delivery system 1 shown in FIG. 1a, the gas supply or gas source 2, 3 is in fluid communication with the gas control unit 8. The gas control unit 8 typically comprises a gas regulator and flow meter. The gas control unit 8 may be an insufflator. The gas control unit 8 may provide continuous or intermittent flow of gases. The continuous or intermittent flow of gases may be at a desired flow rate.

The gas control unit 8 is in fluid communication with the conditioning source 7, shown in FIG. 1A as a humidifier. The conditioning source 7 may condition the gas flow by providing heating and/or humidification to the gas flow. The conditioning source 7 may include a humidifier, a heater, or a humidifier and heater. The humidifier, heater, or humidifier and heater can be connected to or in fluid communication with the patient interface via patient interface conduit or insufflation tube 11.

The gas supply or gas source 2, 3 and control unit 8 can be in fluid communication with the conditioning source 7 via conduits 9, 10. The flow communication can be a serial connection. The connection may include a connector assembly that connects the gases supply or gases source 2, 3 to the gas control unit 8 and conditioning source 7. For example, the connector assembly may be a screw connector, friction connection, male-female lock connector or any other type of connection known in the art.

The conditioning source 7 may be a humidifier. The gases can be humidified as they pass through the humidifier, which can contain a volume of humidification fluid, such as water. The humidified gases can exit out through the humidifier and into the patient interface conduit/insufflation tube 11. The gases can move through the patient interface conduit/insufflation tube 11 into the surgical cavity 6 of the patient via the patient interface 5.

The conditioning source 7 may be a heater. The gases can be heated as they pass through the heater. The heating of the gas flow may reduce or prevent condensation and may assist in keeping the patient at a desired temperature. The gas may be heated using an appropriate means such as heating element. The heating element may comprise a heater plate. The heated gases can exit out through the heater and into the patient interface conduit 11. The gases can move through the patient interface conduit/insufflation tube 11 into the surgical cavity 6 of the patient via the patient interface 5.

The gases can be humidified and heated as they pass through the conditioning source 7. The conditioning source 7 can comprise a humidifier that both humidifies and heats the gas, as shown in FIG. 1a. The humidifier may be any humidifier suitable for surgical applications, such as the SH870 Surgical Humidifier from Fisher and Paykel Healthcare. Such humidifiers typically comprise a humidifier chamber to hold a volume of humidification fluid and a heating system which may include a heater plate. The humidified and heated gases can exit out through the conditioning source 7 and into the patient interface conduit/insufflation tube 11. The gases can move through the patient interface conduit/insufflation tube 11 into the surgical cavity 6 of the patient via the patient interface 5. The patient interface conduit/insufflation tube 11 may be heated to maintain humidity and/or temperature levels in the gas as the gas passes through the patient interface conduit/insufflation tube 11.

Environments within a surgical cavity 6 can include cells that are susceptible to damage when exposed to relatively dry and cold gases. Therefore, the application of warming, humidification, or warming and humidification of the insufflation gas flow may reduce or prevent cellular desiccation. This may have a positive effect on patient outcomes and can increase quality of care, reduce recovery time and reduce length of patient hospital stay. Warming and/or humidifying insufflation gases may also reduce intra-operative hypothermia, reduce post-operative complications and improve post-operative recovery.

Some embodiments described provide a surgical humidification system that includes a humidifier control system configured to determine a mode of operation, a mode of control, a heater plate set point, or any combination of these. The humidifier control system can base this determination at least in part on feedback from components of the humidification system. The components of the humidification system can provide feedback through sensors or other electrical components, and feedback can include, for example, outlet and/or inlet gas temperature, ambient temperature, heater plate temperature, heater plate power, gas flow rate, user input through user interface elements, duration of operation, and the like. Some embodiments of the humidifier control system can improve efficiency of the humidification system, provide an output gas with relatively consistent humidity and/or temperature, and provide greater control over temperature and/or humidity of the gas compared to control systems that do not incorporate system component feedback. The humidifier control system can provide at least some of these improvements through modules configured to process system component feedback and adjust output settings according to a control loop feedback mechanism.

The pressure of the gas flow is preferably such that it drives the flow of gas at operational parameters (such parameters are described below). The flow rate of the gas may contribute to how effectively the surgical cavity is filled with gas.

One or more medicaments may be entrained into the gas flow. For example, the medicament may be entrained into the gas flow as a mist or aerosol. The medicament can be entrained into the gas flow in the delivery assembly 4. For example, the medicament may be entrained into the gas flow by being injected into a delivery conduit 9, 10, 11.

Referring to FIG. 1B, there is shown a configuration of a medical gases delivery system 1 in which the gas source may be provided by a flow generator 17. The flow generator 17 may provide the gas instead of the gas source 2, 3. Alternatively the flow generator 17 may provide the gas in combination with the gas source 2, 3.

A flow generator 17 delivers a flow of gas at a desired pressure and/or flow to the patient interface 5. Use of a flow generator 17 may require fewer devices to provide a source of gas to the surgical cavity 6. The flow generator 17 may comprise a motor (with a pump or compressor) that can draw in ambient or room air, and optionally a secondary gas source (e.g. oxygen or CO₂), which may be provided by the gas supply or gas source 2, 3. The flow generator 17 may alternatively draw gas primarily or solely from the gas supply or gas source 2, 3. If a secondary gas source is supplied to the flow generator 17, ambient or room air and the secondary gas source may be mixed in the flow generator 17 as the gases flow past one or more sensors, including temperature, flow and oxygen sensors. The flow generator 17 operates to deliver gases to the patient interface 5, and optionally via a humidifier.

The flow generator 17, as the gas supply or gas source, may include an integrated humidifier, heater, or humidifier and heater. For example, the flow generator 17 may include a humidifier having a heater plate or other suitable heating element(s) for heating humidification liquid in the humidifier chamber for use during a humidification process. The heater plate can be in thermal communication with the humidifier heating element. The humidifier heating element may therefore transfer heat to the heater plate. The heater plate can thereby transfer heat from the humidifier heating element to the humidifier chamber.

The humidifier of the flow generator 17 may be controlled by at least one controller, which allows for monitoring and control of various flow and/or pressure parameters. At least one controller controls the humidifier, if present, to humidify the gas flow and/or heat the gas flow to a desired temperature. The controller can be programmed with or can determine a suitable target temperature and/or humidity of the gas flow. The controller can be programmed to, or can determine, a suitable target temperature and/or humidity of the gas flow and use one or more of the heating element(s), humidifier heating element and the flow generator to control flow and/or pressure to the target temperature and/or humidity.

The flow generator 17 may include a user interface (comprising, for example, a display and input device(s) such as button(s), a touch screen, or the like). The flow generator controller may be configured or programmed to control the components of the system, such as operating the flow generator 17 to create a flow of gas (gas flow) for delivery to the patient interface 5, operating the humidifier (if present) to humidify and/or heat the generated gas flow, receive one or more input/s from sensors (such as flow, temperature, humidity, and/or pressure) and/or the user interface for reconfiguration and/or user-defined operation of the flow generator, and output information (for example on the display of the device) to the user. The flow generator 17 may include the gas control unit 8 or have gas control unit functionality built into its controller or control system

### 2. Delivery Assembly

The delivery assembly 4 can direct gas flow from gas supply or gas source 2, 3 to the patient interface 5 at a desired flow rate, and at a pressure sufficient to deliver the desired flow rate. Broadly speaking, the delivery assembly 4 comprises one or more conduits 9, 10, 11 adapted to connect to the gas supply or gas source 2, 3, and to connect to the patient interface 5. The delivery assembly 4 may also include a conditioning source 7 (such as a humidifier, heater, or humidifier and heater) and/or gas control unit 8 which may comprise a gas regulator and/or flow meter.

The delivery assembly 4 may include a stand-alone humidifier 7. For example, the humidifier 7 may receive gas flow from the gas control unit 8, which receives gas flow from the gas supply or gas source 2, 3.

In one configuration, the gas control unit 8 is a separate unit to the humidifier 7. In an alternate configuration, the gas control unit 8 may be integrated with the humidifier 7, such as is shown in FIG. 1B. The gas control unit 8 may provide a continuous or intermittent flow of gases. The continuous or intermittent flow of gases may be at a desired flow rate.

In relation to the conduit 9, 10, 11, where the medical gases delivery system 1 comprises a gas supply or gas source 2, 3 and patient interface 5, the conduit 9, 10, 11 may be a single conduit between the gas supply or gas source 2, 3 and patient interface 5. In configurations where the gas supply or gas source is a flow generator 17 that optionally comprises a heater and/or humidifier, the conduit may comprise a conduit between the flow generator 17 and patient interface 5 and between flow generator 17 and gas source. In configurations comprising a flow generator 17 that can draw in ambient or room air, the conduit 9, 10, 11 may be a single conduit between the flow generator 17 and patient interface 5.

A flow generator 17 delivers a flow of gas at a desired pressure and/or flow to the patient interface 5. Use of a flow generator 17 may require fewer devices to provide a source of gas to the surgical cavity 6. The flow generator 17 may comprise a motor (with a pump or compressor) that can draw in ambient or room air, and optionally a secondary gas source (e.g. oxygen or CO₂), which may be provided by the gas supply or gas source 2, 3. The flow generator 17 may alternatively draw gas primarily or solely from the gas supply or gas source 2, 3. If a secondary gas source is supplied to the flow generator 17, ambient or room air and the secondary gas source may be mixed in the flow generator 17 as the gases flow past one or more sensors, including temperature, flow and oxygen sensors. The flow generator 17 operates to deliver gases to the patient interface 5, and optionally via a humidifier.

Where the medical gases delivery system 1 comprises a conditioning source 7 or gas control unit 8, the conduit 9, 10, 11 may comprise a conduit 9 between the gas supply or gases source 2, 3 and the conditioning source 7 or gas control unit 8 (i.e. a gas supply conduit 9). The medical gases delivery system 1 may have a further conduit 11 (patient interface conduit/insufflation tube) between the conditioning source 7 or gas control unit 8 and the patient interface 5 (i.e. a patient interface conduit 11/insufflation tube).

Where the medical gases delivery system 1 comprises a conditioning source 7 and gas control unit 8, the conduit 9, 10, 11 may comprise:
a conduit between the gas supply or gas source 2, 3 and the gas control unit 8 (i.e. a gas supply conduit 9),
a conduit between the gas control unit 8 and the conditioning source 7 (i.e. an intermediate conduit 10), and
a conduit between the conditioning source 7 and the patient interface 5 (i.e. a patient interface conduit or insufflation tube 11).

A delivery assembly 4 having the gas control unit 8 upstream from the humidifier 7 is preferred so that the humidifier chamber is subjected to lower pressure. It is typically preferable to locate the control unit 8 upstream from the conditioning source 7 (humidifier) so the humidifier chamber of the conditioning source 7 is subjected to lower pressure.

In some embodiments the conditioning source 7 may be upstream of the gas control unit 8.

The delivery assembly 4 is configured to provide a pneumatic connection between the gas source 2, 3 and the patient interface 5. The required pressure must be sufficient to overcome the inherent resistance to flow in the delivery assembly 4 and the patient interface 5.

The delivery assembly 4 may include a heated conduit to reduce internal condensation, such as by a heating element that extends through at least part of the delivery assembly conduit 9, 10, 11. The conduit 9, 10, 11, or one or more sections of the conduit (such as the patient interface conduit/insufflation tube 11) may be heated.

For example, in the presence of a conditioning source 7 including a humidifier, the conduit between the conditioning source 7 and the patient interface 5 (i.e. the patient interface conduit 11) may solely be heated. However, each of the conduits 9, 10, 11 may be heated as required.

The heated conduit may comprise a tube having one or more conductive elements embedded, encapsulated or otherwise located in the tube. The one or more conductive elements can be heating filaments (or more specifically, resistance heating filaments). The patient interface 5 may removably or permanently attach to the delivery assembly 4.

The gas control unit 8 may include one or more sensors such as a pressure sensor or flow sensor. The gas control unit 8 may control the pressure and/or flow of the gas being delivered to the patient interface 5. The gas control unit 8 may include a pressure regulator that reduces the input pressure of the gas flow. The gas control unit 8 may mix one or more gas sources and/or gas flow rates. The gas control unit 8 may be located between the gases supply or gases source and the patient interface 5.

The conduit 9 extending between the gas supply or gas source 2, 3 and the gas control unit 8 may be a high-pressure conduit. The high-pressure conduit may have an operating pressure rating of about 200 bar. The operating pressure of the high pressure conduit may depend on one or more of the type and source of gas and the procedure that the gas is being used for. For example, CO₂ bottles are typically 50 bar and wall supply CO₂ is typically less than 10 bar.

In some configurations, the conduit 9, 10, 11 comprises an electrical connector. The electrical connector may be coupled to one or more sensors and/or power connectors. In some configurations, the electrical connector projects from a portion of the delivery assembly 4.

In some configurations, the electrical connector of the conduit 9, 10, 11 is coupled to one or more sensors. In one embodiment the sensor may be one or more of a temperature, flow or humidity sensor. The sensor(s) may be configured to measure temperature, flow and/or humidity of gas flowing through the delivery assembly 4 at the location of the sensor(s).

### 3. Patient Interface

As discussed above, the medical gases delivery system 1 comprises a delivery assembly 4 for receiving gases from the gas supply or gas source 2, 3 and directing the gases to the patient interface 5.

The patient interface 5 is attachable to the outlet end of the delivery assembly 4, that being the end of the delivery assembly 4 proximal to the patient interface 5. For example, the patient interface 5 can be connected to the patient interface conduit/insufflation tube 11. The patient interface 5 may be attached with any suitable connection means, such as, for example a luer, screw thread or friction fit connection.

Where the delivery assembly 4 comprises a conditioning source 7 (such as a humidifier and/or heater) and/or gas control unit 8 the patient interface 5 is connected to patient interface conduit/insufflation tube 11 that delivers gas flow to the patient interface 5.

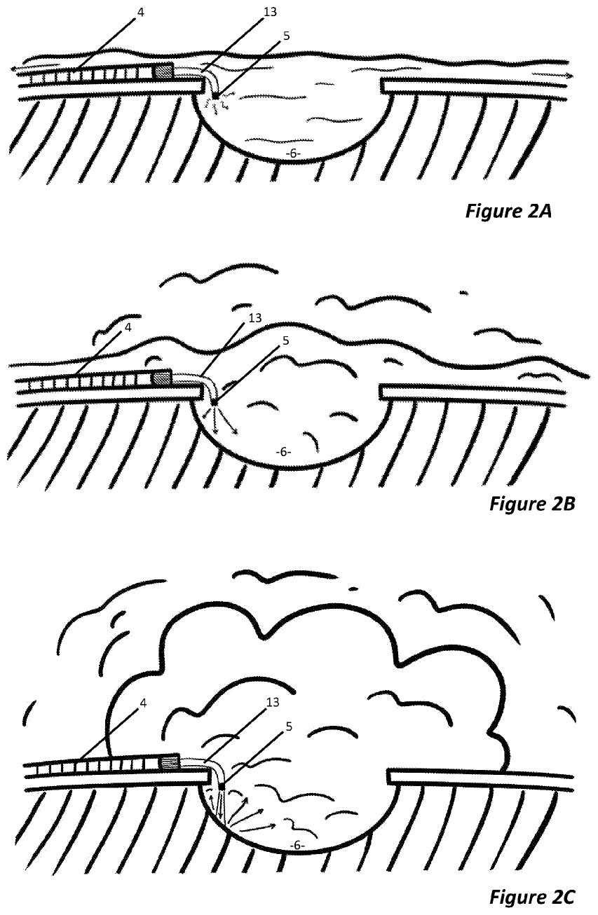

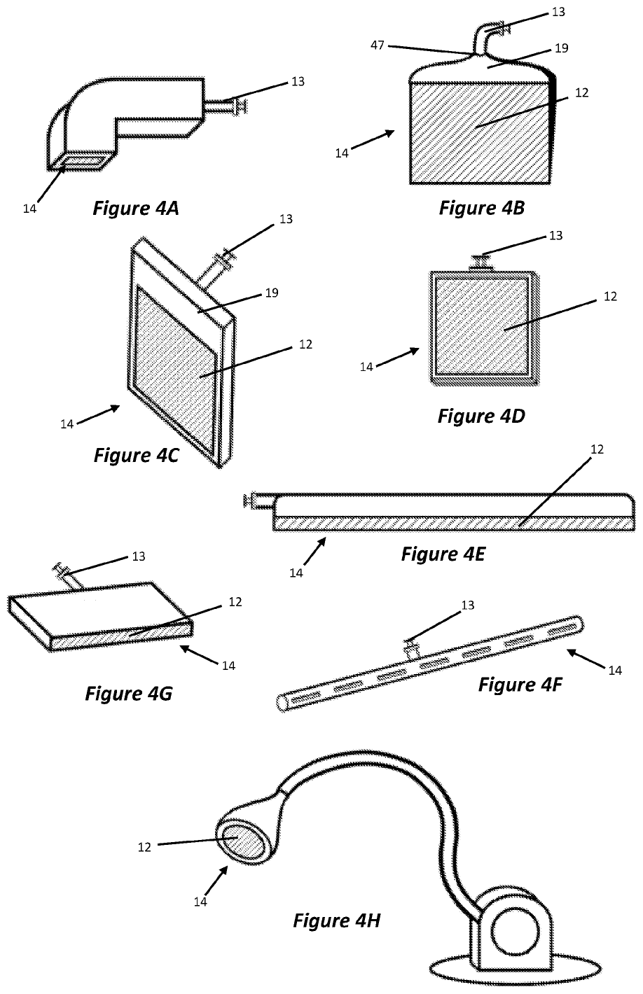

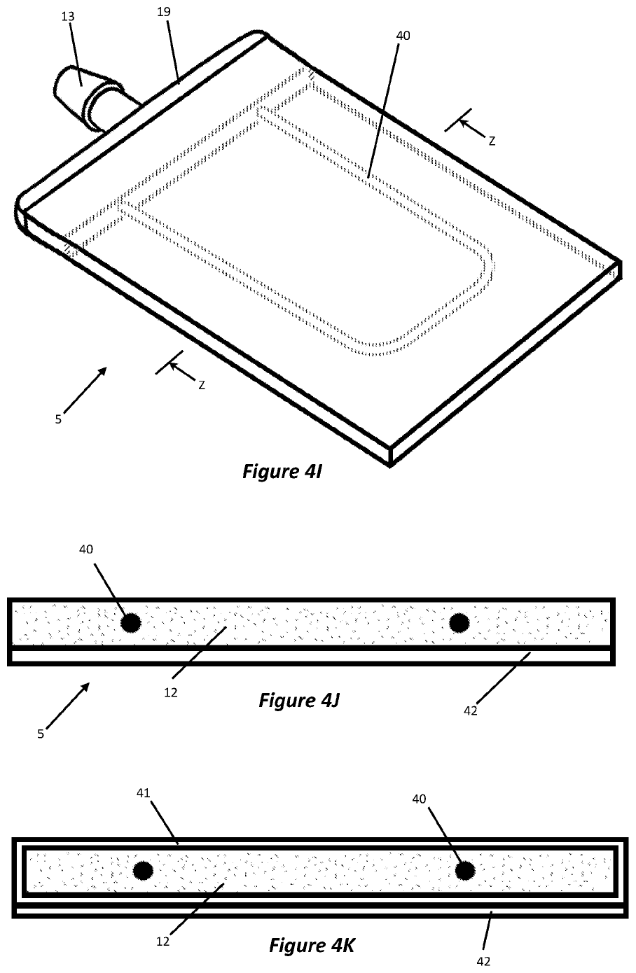

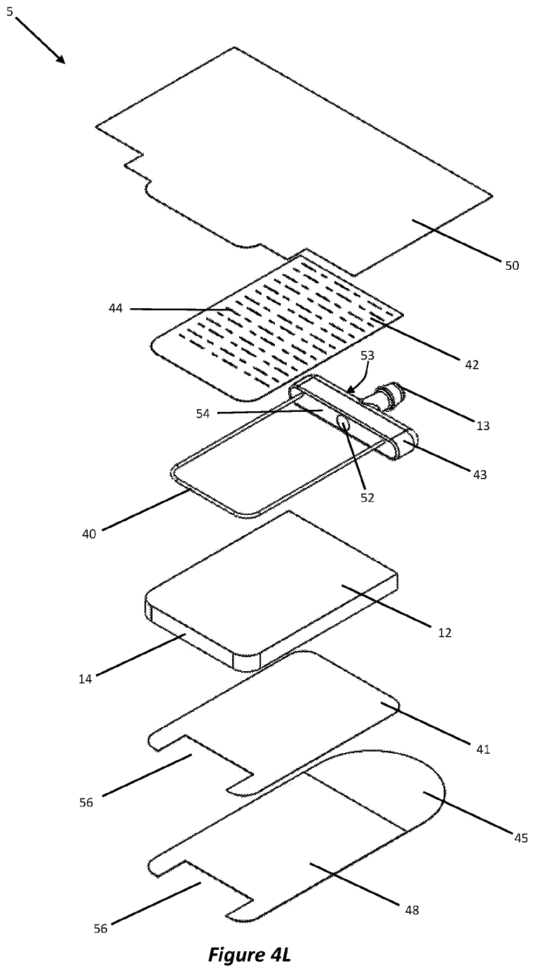

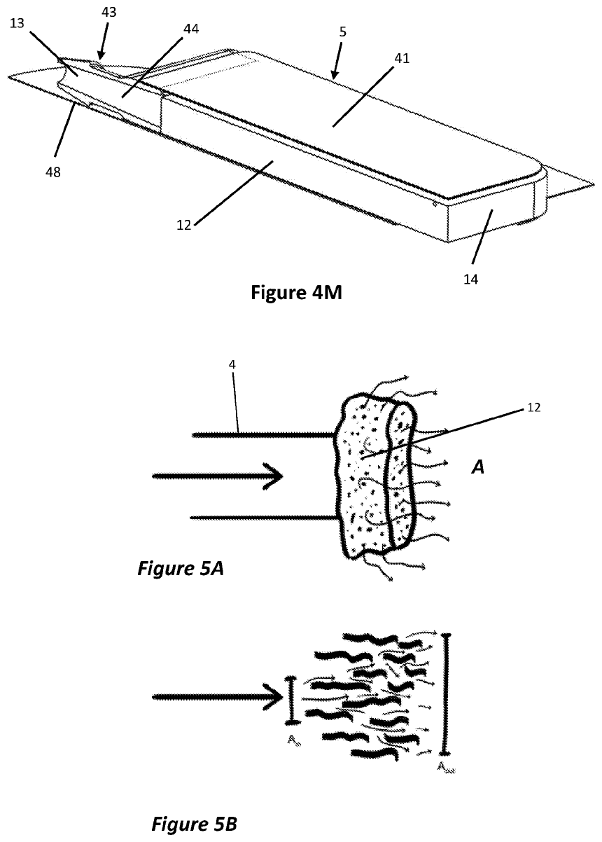

As shown in, for example, FIGS. 2A-C, 8C, 8D, 9A, 9B, 11, 12A, 16A and 19-23, the patient interface 5 may include an interface inlet tube 13. As shown in FIG. 4B the interface inlet tube 13 may connect to an interface inlet 47 at one end. The interface inlet tube 13 may then attach to the interface tube/insufflation tube 11 of the delivery assembly 4. The connection to the interface inlet 47 and the interface tube/insufflation tube 11 may be with any suitable connection means, such as, for example a luer, screw thread or friction fit connection. In some embodiments the interface inlet tube 13 may be integrally formed with the patient interface 5 and connect to the interface tube/insufflation tube 11 with any suitable connection means, such as, for example a luer, screw thread or friction fit connection. In one embodiment, the patient interface 5 may be integrated with the delivery assembly 4.

The use of an interface inlet tube 13 may provide for the delivery assembly 4 to be spaced from the surgical cavity 6 as the connection point between the interface inlet tube 13 and the delivery assembly 4 may be spaced from the surgical cavity 6. Such a design may assist in keeping the connection point and/or patient interface conduit/insufflation tube 11 out of, or away from the surgical cavity 6, thereby avoiding the connection point or patient interface conduit 11 taking up space within or impeding access to the surgical cavity 6.

The interface inlet tube 13 may be formed from a soft and/or flexible material such that the interface inlet tube 13 can be manipulated to form any shape but does not have to maintain that shape. Alternatively, the interface inlet tube 13 may be formed of a deformable material that is resilient, such that the material retains its shape when a force is applied.

In some embodiments the patient interface 5 and the interface inlet tube 13 may be integrally formed with the patient interface conduit 11/insufflation tube. In some embodiments, the patient interface 5 may be integrally formed with the patient interface conduit 11/insufflation tube.

The patient interface 5 reduces the velocity of the gas at the patient interface outlet 14 such that it fills the surgical cavity 6. Reduction of velocity of gas at the outlet 14 may influence the nature of the gas flow as it exits the outlet. For example, turbulence may be reduced and/or the gas may exit the outlet 14 as a substantially laminar flow. Depending on the configuration of the patient interface 5, including the configuration of the outlet 14, gas may exit the outlet 14 as a unidirectional, bidirectional or omnidirectional gas flow. The patient interface 5 preferably provides a substantially uniform and even fill of the surgical cavity 6, with gradual flowing/flooding of gas over the upper edge of the surgical cavity 6 as shown in FIG. 2A.

A substantial portion of the gas may exhibit laminar flow as it fills the surgical cavity 6. As used herein the term “laminar flow” means that there is not substantial disruption between layers of gas flow. As can be seen in FIG. 2A, there is no or negligible distribution of gas within the operating theatre. In other words, some gas may exit the surgical cavity 6, but any gas that exits is not distributed in or around the room. Gas that exits the surgical cavity 6 does so in a controlled manner and if the gas is denser than air, the gas exits and falls downwards. The gas is thus substantially localized to the surgical cavity 6. This may be advantageous not only in terms of forming a partial protective environment in the surgical cavity 6, but also in minimizing dispersion and distribution of fluid droplets out from the surgical cavity 6. Minimizing or otherwise reducing the release of droplets, particularly aerosolized droplets from the surgical cavity may be of particular use in mitigating risk of infection to the surgeon and other operating staff in situations where the patient has an infectious agent, e.g. virus, in their system.

Without wishing to be bound by theory, if the velocity of the gas at the patient interface outlet 14 is too high, jetting may occur and the gas may not form a partial protective environment in the surgical cavity 6. For example, as shown in FIG. 2B there is still a small amount of jetting, but the surgical cavity 6 is still filled with the gas with some overflow flooding. Uniformity of gas distribution in the surgical cavity 6 is not, however, as consistent as that shown in FIG. 2A.

Jetting may lead to turbulent mixing which may result in a poorly filled surgical cavity 6 as shown in FIG. 2C. FIG. 2C demonstrates a failure of filling where jetting occurs. The gas does not form a uniformly distributed body of gas in the surgical cavity 6 and there is no overflow flooding. Thus, jetting may reduce the ability of the gas to provide any therapeutic benefits to the patient.

The density of the gas provided by the gas source or gas supply 2, 3 may be greater than air. A gas with a density higher than air may assist distributing, settling and/or maintaining the gas in the surgical cavity 6, particularly where the patient interface 5 has an outlet 14 that is at, adjacent, or above, the surgical cavity 6.

In some embodiments the gas may have a density the same, or similar, to air. A gas having a density the same, or similar, to air may be used in conjunction with an interface located in the surgical cavity 6, such that the interface outlet 14 is located within the surgical cavity 6.

The flow rate of the gas in the delivery assembly 4 and/or conduit 8 may be less than 20 L/min, or less than less than 15 L/min, or less than 10 L/min, or less than 9 L/min, or less than 8 L/min, or less than 7 L/min, or less than 6 L/min, or less than 5 L/min, or less than 4 L/min, or less than 3 L/min, or less than 2 L/min, or less than 1 L/min L/min, or less than 0.5 L/min.

The velocity of the gas flow at the patient interface outlet 14 may be less than 2.0 ms⁻¹, or less than 1.5 ms⁻¹, or less than 1.0 ms⁻¹, or less than 0.9 ms⁻¹, or less than 0.8 ms⁻¹, or less than 0.7 ms⁻¹, or less than 0.6 ms⁻¹r, or less than 0.5 ms⁻¹, or less than 0.4 ms⁻¹. The gas flow velocity at the outlet 14 is important to avoid jetting and mixing. The patient interface outlet 14 gas flow is reduced by the passage of the gas flow through or across the patient interface 5. The velocity of gas flow at the patient interface outlet 14 is thus less than the velocity of gas flow entering the inlet tube 13 and/or patient interface inlet. The design of the patient interface 5 is such that it distributes the flow over a large surface area, reducing the velocity of the flow. Preferably the surface area is at least 1110 mm². A range of designs can be used to achieve this, which are described below.

The volumetric flow rates through the medical gases delivery system 1 may be about 0.1, 1, 2, 3, 4, 5, 6, 7, 8, 9, 10, 11, 12, 13, 14, 15, 16, 17, 18, 19 or 20 L/min, and useful ranges may be selected between any of these values (for example 0.1 to about 20, about 0.1 to about 150.1 to about 12, about 0.1 to about 10, about 0.1 to about 5, about 1 to about 20, about 1 to about 16, about 1 to about 13, about 1 to about 10, about 1 to about 8, about 2 to about 20, about 2 to about 15, about 2 to about 10, about 3 to about 20, about 3 to about 17, about 3 to about 12, about 3 to about 10, about 4 to about 20, about 4 to about 13, about 4 to about 10, about 5 to about 20, about 5 to about 16, about 5 to about 12, about 5 to about 10, about 6 to about 20, about 6 to about 15, about 6 to about 13, about 6 to about 10, about 7 to about 20, about 7 to about 16, about 7 to about 10, about 8 to about 20, about 8 to about 14, about 8 to about 10, about 9 to about 20, about 9 to about 15, about 10 to about 20, about 10 to about 15 or about 15 to about 20 L/min).

The velocity of the gas flow into the surgical cavity 6 is about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 m/s, and useful ranges may be selected between any of these values (for example 0.05 to about 2.0, about 0.05 to about 1.5, about 0.05 to about 1.0, about 0.1 to about 2.0, about 0.1 to about 1.6, about 0.1 to about 1.0, about 0.1 to about 0.8, about 0.2 to about 2.0, about 0.2 to about 1.4, about 0.2 to about 1.0, about 0.2 to about 0.8, about 0.3 to about 2.0, about 0.3 to about 1.6, about 0.3 to about 1.0, about 0.3 to about 0.8, about 0.4 to about 2.0, about 0.4 to about 1.6, about 0.4 to about 1.0, about 0.4 to about 0.8, about 0.5 to about 2.0, about 0.5 to about 1.6, about 0.5 to about 1.0, about 0.6 to about 2.0, about 0.6 to about 1.6, about 0.6 to about 1.0, about 0.7 to about 2.0, about 0.7 to about 1.6, about 0.8 to about 2.0, about 0.8 to about 1.6, about 0.8 to about 1.0, about 0.9 to about 2.0, about 0.9 to about 1.6, about 1.0 to about 2.0, about 1.0 to about 1.6, about 1.1 to about 2.0, about 1.1 to about 1.6, about 1.2 to about 2.0 or about 1.2 to about 1.6 m/s).

In one embodiment the patient interface 5 slows the gas flow entering the surgical cavity 6 by converting a portion of the flow's kinetic energy into potential energy of pressure.

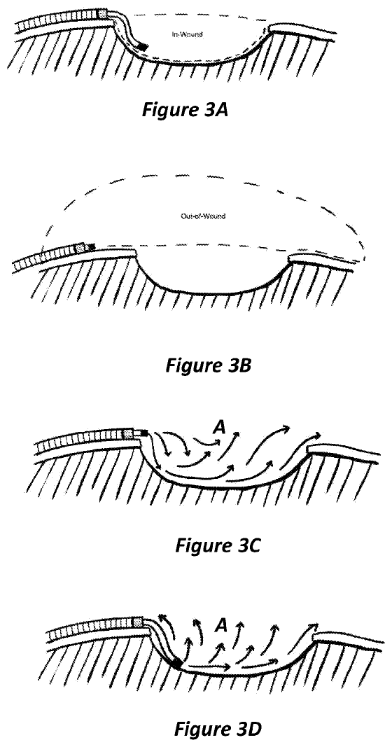

In one embodiment the patient interface 5 is placed inside the surgical cavity 6 as shown in FIG. 3A. In such an embodiment the gas flow is delivered directly into the surgical cavity 6. In such an embodiment, density of the gas is preferably at least the same, or similar, to air.

In an alternative embodiment, the patient interface 5 may be placed adjacent to the surgical cavity 6 as shown in FIG. 3B. In such an embodiment, density of the gas is preferably greater than air. In such an embodiment, the patient interface 5 may be more adaptable to variations in the volume and/or geometry of the surgical cavity, less obtrusive in the surgical working space, and/or may limit interactions between the patient interface 5 and body fluids.

In embodiments where the patient interface 5 is configured to be placed adjacent to the surgical cavity 6, the interface 5 may be configured to direct a flow of gas across the surgical cavity 6. In other words, gas flow may be directed outwardly of the outlet 14 from one side of the surgical cavity 6 to an opposite side thereof. The gas flow may prevent entry of ambient air into the gas flow and/or into the surgical cavity 6. In such embodiments, the patient interface 5 may be placed on a surface adjacent to where a surgical incision is to be made. The interface 5 may be placed on a surface of the patient skin, on film/drape, placed over the patient skin or similar. The interface 5 may be placed such that the interface 5 is close enough to the surface to achieve attachment of a boundary layer of flow of gas toward the surgical site/surgical cavity 6. The flow of gas may be substantially non-turbulent or laminar as it exits the outlet 14 of the interface 5. The flow of gas may be substantially unidirectional, i.e. flows outwardly from the outlet 14 in substantially one direction only.

A substantially non-turbulent or laminar and/or unidirectional flow can be formed by features of the patient interface 5, as will be described below. In such embodiments, the interface 5 may be placed close enough to the surface to achieve attachment of a boundary layer of unidirectional and/or non-turbulent or laminar flow of gas toward the surgical site/surgical cavity 6 along substantially the width of the interface 5 or the outlet 14. In such an embodiment, airborne particles may be prevented from entering the gas flowing out of the outlet 14 and over the surgical cavity 6. Air and airborne particles may be prevented from being entrained into the gas flow or beneath the gas flow and into the surgical cavity 6. The flow of gas may provide a protective layer or seal over the surgical cavity 6. The protective layer may be formed with or without filling of the surgical cavity 6 with the gas.

The position of the patient interface 5 may govern how the surgical cavity 6 is filled. The surgical cavity 6 may be filled from the top of the surgical cavity 6 (top-down filling) as shown in FIG. 3C. This may mitigate the possibility of gas pumping/moving internal organs, blood/body fluid splatter and/or undesirable noise.

Gas pumping or organ pumping can occur where the outlet of the patient interface 5 becomes located in gaps between the internal anatomy and body tissues. This may create an inflatable pocket to push body tissue apart and open the pocket to release the now pressurised gas. As the pressure drops, the pocket may contract back to its original shape, causing the body tissue to move back and recreate the sealed pocket. Movement of body tissue may hinder or at least inconvenience surgeons' ability to carry out surgical procedures.

Alternatively, the surgical cavity 6 may be filled from the bottom (bottom-up filling) as shown in FIG. 3D. Bottom-up filling may assist in eliminating the possibility of entrainment. Entrainment can occur when a shear force is induced between the environment and fluid stream when a stream of fluid moves through an ambient environment. The shear force causes some of the ambient environment to be pulled in the direction of the moving fluid stream, which is known as entrainment. In the context of a gas being delivered into a surgical cavity, entrainment is the unintentional transport of the ambient air (that is adjacent to the therapeutic gas stream) into the surgical cavity. This may have the undesirable consequence of pulling airborne contaminants (such as dust) into the surgical cavity.

In top-down filling, gas is delivered above, at the top of, or adjacent an upper edge of the surgical cavity 6. The gas then floods down into the surgical cavity 6 to fill. In bottom-up filling, gas is delivered towards the bottom of the surgical cavity 6 and the gas fills the surgical cavity 6.

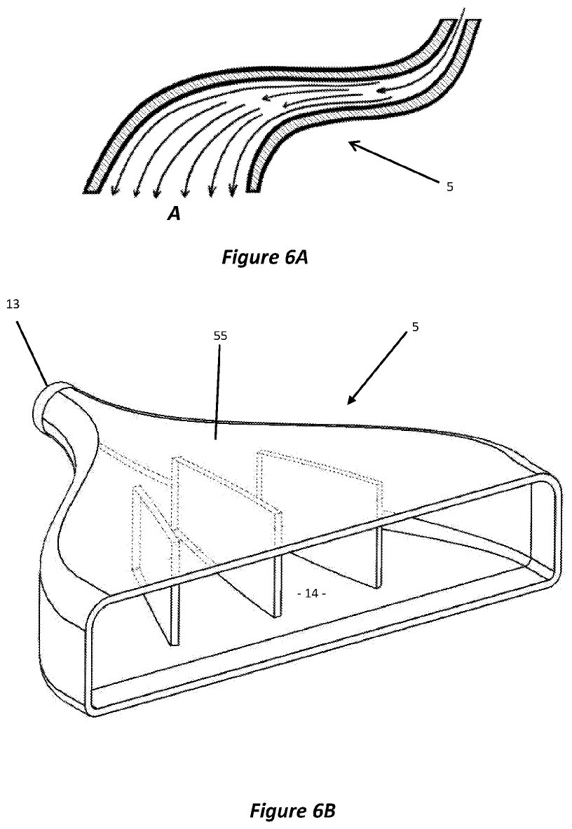

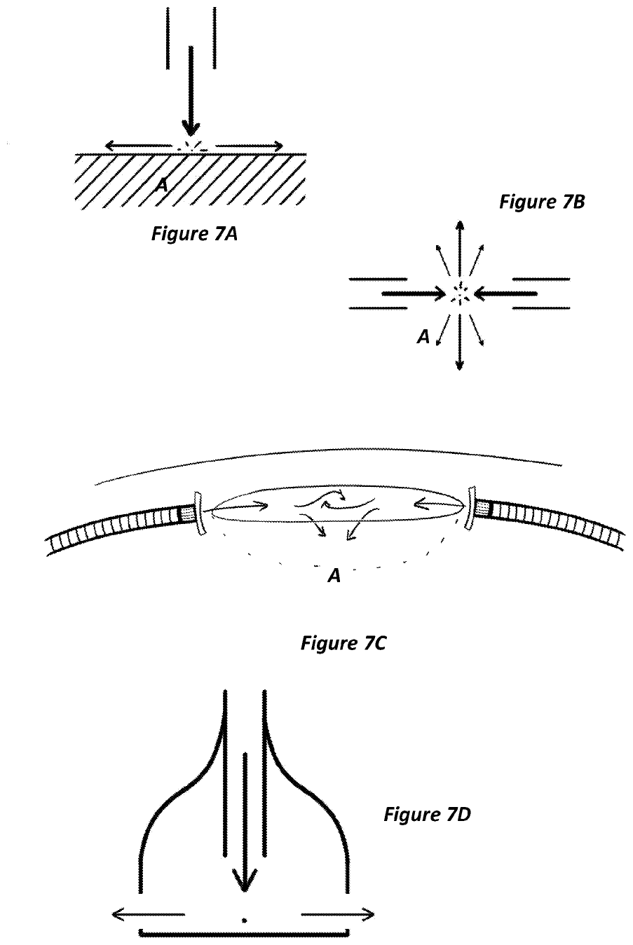

It may be advantageous to use a patient interface 5 with an outlet surface area of at least 180, 185, 190, 195, 200, 205, 210, 215, 220, 225, 230, 235, 240, 240 or 250 mm², and suitable ranges may be selected from between any of these values, (for example, about 180 to about 250, about 180 to about 230, about 180 to about 200, about 185 to about 250, about 185 to about 235, about 185 to about 215, about 190 to about 250, about 190 to about 240, about 190 to about 200, about 195 to about 250, about 195 to about 225, about 195 to about 215, about 200 to about 250, about 200 to about 235, about 200 to about 210, about 205 to about 250 or about 205 to about 240, about 210 to about 250 mm²). The surface area of the outlet may be calculated based on the physical surface area of the patient interface outlet 14 as shown in FIGS. 4A to 4H. Alternatively, the outlet surface area may be calculated on the notional surface area of flow. That is, where the interface leads to a flow that has a much broader range of flow directions compared to the unidirectional flow of the gas prior to traversing the interface. This is demonstrated in FIGS. 7A to 7D (discussed below) where the initial gas flow direction has a linear unidirectional flow, and after traversing the patient interface 5 the direction of gas flow is much more diverse and distributed.

In one embodiment the gas pressure at the patient interface outlet 14 is less than 30 mmHg. The gas pressure at the interface outlet may be less than 28 mmHg. The gas pressure at the interface outlet may be less than 26 mmHg. The gas pressure at the patient interface outlet 14 may be less than 24 mmHg. The gas pressure at the patient interface outlet 14 may be less than 22 mmHg. The gas pressure at the patient interface outlet 14 may be less than 20 mmHg. The gas pressure at the patient interface outlet 14 may be less than 18 mmHg. The pressure at the patient interface outlet 14 may be measured by a sensor, such as a pressure sensor. The pressure sensor may generate a signal that is sent to a controller for closed loop control. The controller may increase or decrease the flow of gas to maintain the desired outlet pressure. Alternatively, the controller may increase or decrease the pressure of gas to maintain the desired outlet flow.

The flow rate of the gas through the medical gases delivery system 1 may define at least in part how effectively the surgical wound is filled. The pressure has to be sufficient to deliver the desired flow rate. The flow rate may define at least in part how fast the surgical cavity 6 is filled. Flow rate can be adjusted to allow gas to exit from the interface 5 at a selected velocity and/or as a substantially laminar or non-turbulent flow.

In some embodiments the patient interface 5 comprises a support structure 19 having a connector inlet 47 and a connector outlet 52. The connector inlet 47 of the support structure 19 provides an attachment or coupling means to the interface inlet tube 13. In some embodiments the inlet 47 may include a barbed portion for attachment to the interface inlet tube 13. In alternate embodiments the interface inlet tube 13 may be formed integrally with the support structure 19.

In some embodiments, the support structure 19 can provide an attachment, coupling means and/or housing for a gas-permeable substrate 12. In some embodiments, the support structure 19 may comprise, or define, a chamber 55. In some embodiments, the support structure 19 may comprise a passage between the connector inlet 47 and the connector outlet 52 to allow passage of the flow of gas to the gas-permeable substrate 12. In some embodiments the support structure 19 may be in the form of, or define, a frame for the gas-permeable substrate 12. The support structure 19 may abut, adjoin, comprise or at least partially house the gas-permeable substrate 12, which provides a conditioned flow of gas. Without wishing to be bound by theory the gas-permeable substrate 12 provides a diffuse flow of gas from the interface outlet 14. The gas-permeable substrate 12 may fill a portion of the support structure 19 of the patient interface 5. The support structure 19 may comprise a chamber 55 between the gas-permeable substrate and the connector inlet 47. Alternatively, the entirety of the support structure may be filled with the gas permeable substrate 12.

The gas-permeable substrate 12 allows for gas to pass through the substrate 12 and transforms the inlet gas flow to a diffuse flow into the surgical cavity 6. The gas-permeable substrate 12 may be selected from any material and/or structure that is porous, for example, sintered plastics, open cell foam and other porous permeable substrates 18, for instance paper, felt, sintered metal and/or filter material.

If present, the gas-permeable substrate 12 may:
be located at, or may at least partly define, the outlet 14 of the patient interface 5 to define a cavity in the support structure between the gas-permeable substrate 12 and the inlet of the patient interface device 5; or
be located at, or proximate to the inlet of the patient interface device 5 to define a cavity in the support structure between the gas-permeable substrate 12 and the outlet 14 of the patient interface device 5; or
be located in the patient interface device 5 to define cavities in the support structure between the gas-permeable substrate 12 and both the inlet and the outlet of the patient interface device 5.

The cavity within the support structure 19 located upstream of the gas-permeable substrate 12 may allow for the gas flow to distribute about the surface of the gas-permeable substrate 12 providing a larger surface area of gas-permeable material through which the gas flow passes.

Shown in FIGS. 4A and 4C to 4H are examples of patient interfaces 5 wherein the support structure 19 comprises a chamber 55. The chamber of the support structure 19 may comprise or house a gas permeable substrate 12. The entirety of the chamber 55 may be filled with the gas-permeable substrate 12, or a portion thereof.

Gas flow entering the chamber 55 may be via the interface inlet tube 13 or the patient interface conduit 11. The gas flow is conditioned when passing through the gas-permeable substrate 12. For example, the gas flow is conditioned to be a diffuse and/or substantially non-turbulent flow (i.e. a substantially laminar flow) to thereby provide a diffuse and/or non-turbulent gas flow into the surgical cavity 6. The gas flow directed outwardly from the interface outlet 14 may be unidirectional, bidirectional or omnidirectional.

FIGS. 4A to 4F show a patient interface 5 in which the outlet 14 of the patient interface 5 is directed at an angle relative to the direction of inlet gas flow. The offset angle of the outlet relative to the inlet may be between about 45 and about 180 degrees, and more preferably about 120 degrees. The inlet of the patient interface 5, such as the interface inlet tube 13, of FIG. 4B or 4C may swivel relative to the body of the patient interface 5.

In relation to the patient interface 5 of FIG. 4A the angle offset may allow the patient interface device 5 to be positioned close to the wound edge and extend to the upper patient wall with the interface outlet 14 providing a flow of gas downwardly into the surgical cavity 6.

Each of the patient interfaces 5 shown in FIGS. 4B to 4D comprise an interface outlet 14 in which the surface area of the outlet 14 is defined by the gas-permeable substrate 12. The thickness of the gas-permeable substrate 12 may be thin relative to the surface area, the interface providing a large area for the gas flow to distribute over as it passes through the patient interface 5. The patient interfaces 5 of FIGS. 4B to 4D may comprise a gas-permeable substrate 12 that has a thickness less than the thickness of the patient interface 5 thus providing for a support structure between the gas-permeable substrate 12 and the inner back wall of the patient interface 5. In one configuration, the gas-permeable substrate 12 may comprise one or more layers of gas-permeable substrate 12. In one embodiment the inlet gas flow may be directed through and/or between two layers of the gas-permeable substrate 12. There may be a cavity or gap formed between the two layers of the gas-permeable substrate 12. In one embodiment the gas-permeable substrate 12 is fixed to or abuts the support structure 19 (e.g. as seen in FIG. 4B) or a frame (e.g. as seen in FIG. 4C or 4D) of the patient interface 5, for example, along the edge of the gas-permeable substrate 12. This may act to prevent the loss of gas between the support structure 19 of the patient interface 5 and the gas-permeable substrate 12 thus forcing the gas flow to pass through and/or between the gas-permeable substrate 12. The support structure 19 may have a ledge or other surface, extending outwardly from the inlet, onto which the gas permeable substrate 12 may be attached. Attachment of the gas permeable substrate 12 onto a ledge may assist to prevent blockage of an outlet of the support structure 19 providing flow of gases to the gas permeable substrate 12. The gas permeable substrate 12 may sandwich a protrusion (such as a ledge) of the support structure. The gas permeable substrate 12 may form a finger joint, a bridle joint or lap joint with the support structure 19.

As shown in FIGS. 4B and 4C the patient interface 5 may comprise a support structure 19 at the inlet of the patient interface 5. The support structure 19 may have a connector outlet 52 to direct gas flow from the delivery assembly 4 into and through the gas permeable substrate 12.

The patient interface devices 5 of FIGS. 4B to 4D may in use, sit adjacent the upper patient wall of the surgical cavity 6 such that the gas flow is directed across and into the surgical cavity 6.

FIGS. 4C and 4G show a patient interface device 5 in which the interface outlet 14 is substantially in line with direction of the gas flow into the inlet of the patient interface 5. The device of FIGS. 4E and 4G may sit adjacent the wound edge of the surgical cavity to direct a flow of gas across the surgical cavity 6.

As seen in FIGS. 4E and 4F, the outlet 14 may be one or more elongate slits that comprises the gas-permeable substrate 12, such that the gas flow must pass through the gas-permeable substrate 12 to vent from the patient interface 5. A cavity in the support structure may be provided between the gas-permeable substrate 12 and the gas flow inlet to allow gas flow to distribute the full length of the slit to increase the available surface area of the gas-permeable substrate 12 that gas flow can pass through. As seen in FIG. 4F the elongate slit may comprise a series of smaller slits.

FIG. 4H shows a patient interface 5 with a support structure that expands from the inlet through to the outlet 14 with the surface area of the interface outlet 14 defined by the gas-permeable substrate 12. There may be provided a cavity in the support structure between the gas-permeable substrate 12 and the gas flow inlet to allow gas flow to distribute across the full surface of the gas-permeable substrate 12.

In one embodiment the support structure is elongate having a body defining a gas inlet end and a distal end. The support structure 19 may comprise plurality of apertures along its length that define the patient interface outlet 14. The apertures may increase in size towards the distal end of the support structure body. The support structure 19 may be configured as a cylinder or a tube of varying cross section along its length.

Referring to FIG. 4B, there is shown a patient interface 5 comprising a support structure 19 in the form of a chamber 55, the support structure 19 having a connector inlet 47 fluidly connected to the interface inlet tube 13. The gas permeable substrate 12 abuts the support structure 19 and has a front and rear face. The patient interface 5 may have sidewalls that can define the thickness of the patient interface 5. The upper side wall of the gas permeable substrate 12 may abut and/or overlap at least a portion of the support structure 19. The gas permeable substrate 12 may be adhered or otherwise affixed to the support structure 19 with adhesive or any other suitable means. The gas permeable substrate 12 may extend at least partly into the support structure 19 to fill a portion of the support structure 19. Alternatively, the gas permeable substrate 12 may abut or overlap a surface of the support structure 19. Gas flows through the support structure 19 from the interface inlet tube 13 and into the gas permeable substrate 12.

The patient interface 5 may include an adhesive layer 42 on the rear surface or patient-facing surface of the patient interface 5. An example is shown in FIG. 4J. The adhesive layer may be a peel-off layer, that is, a layer is removed to expose the adhesive coating. The adhesive layer 42 may coat the rear surface of the patient interface 5, or a portion thereof. When in use, the patient interface 5 may be positioned in the surgical cavity 6 against the patient wall, on a surface adjacent the surgical site/surgical cavity or on a medical instrument such as surgical retractor. The diffused gas flow thus fills or covers the surgical cavity 6 as it flows out of the gas permeable substrate 12 of the patient interface 5. In some embodiments, such as shown in FIG. 4B, substantially the entire surface area of the front face of the gas permeable substrate 12 defines the interface outlet 14.

The support structure 19 may be formed of a rigid material such as plastic, a flexible material such as silicon, or a combination of rigid and flexible materials.

At least a portion of the outer surface area of the gas permeable substrate 12 may be coated, covered or enclosed in an enclosing or outer layer. The enclosing or outer later may be formed from one or more layers. The term “outer layer” does not limit the layer to be the outermost layer of the interface device, merely that the outer layer is external to the gas flow path. The enclosing or outer layer may be at least partly flexible. Flexibility may be imparted by physical properties of the material of the wall and/or from design features of the interface 5, as will be described later in this specification. The enclosing or outer layer may be substantially gas-impermeable, such that gas flow is directed through the interface 5 from inlet, through the gas permeable substrate 12 and exiting via outlet 14. The enclosing or outer layer may include a substantially gas-impermeable layer 41, such as is shown in FIG. 4K. The enclosing or outer layer may include a gas-impermeable membrane, skin or the like. A flexible, substantially gas-impermeable enclosing or outer layer can provide flexibility to the interface 5 while directing gas flow through the interface 5 from inlet 13, through gas permeable substrate 12 and exiting the interface 5 via outlet 14.

The enclosing or outer layer or part thereof may comprise a breathable material. A breathable material in the context of this specification is a material that allows the passage of water molecules through a wall of the material, without allowing the bulk passage of liquid water or bulk flow of gases all the way through the enclosing or outer layer is described as a ‘breathable’ material. Passage of water molecules through such an enclosing wall or outer membrane, such as a monolithic wall, may be via the solution-diffusion mechanism. It may be appreciated by one of skill in the art that the water molecules in the wall are molecularly dispersed in the media and are therefore without a state (solid, liquid or gas), sometimes referred to in the art as vapour. The rate of transfer is often referred to as a water vapour transmission rate or the like.

A ‘breathable’ material may be breathable due to its composition, physical structure or a combination thereof. Examples of breathable materials include block copolymers, hydrophilic polyester block copolymers, thermoplastic elastomers, styrene block polymers, copolyester elastomers, thermoplastic polyolefin elastomers, thermoplastic polyurethane elastomers, non-porous monolithic polymers, polyurethanes, hydrophilic thermoplastics, hydrophilic polyesters, perfluorinated polymers, polyamides, and woven treated fabrics exhibiting breathable characteristics.

The enclosing or outer layer defining at least part of the gas flow path through the interface 5 may be made of the breathable material. A variety of designs of the patient interface 5 utilising the breathable material are possible. For example, the entire enclosing or outer layer of the interface 5 may be formed of a breathable material; a portion of the enclosing or outer layer of the interface 5 may be formed of a breathable material; a portion of the enclosing or outer layer over a top of the interface 5 may be formed of a breathable material. A region or regions e.g. portions of the entire enclosing or outer layer of the gas flow path, of the interface 5 may be formed of the breathable material. The breathable material may be placed over the gas permeable substrate 12 and/or may be bonded or otherwise attached to the gas permeable substrate 12. The interface 5 may comprise one or more layers of breathable material. The breathable material can provide a water vapour flow path from the interface 5 to ambient air.

Breathable regions of the interface 5 may allow diffusion of water vapour from the interface 5 to eliminate or mitigate build up of condensation within the interface 5. The breathable regions may reduce the risk of condensation accumulation and possible saturation of the gas permeable substrate 12.

The enclosing or outer layer defining at least part of the gas flow path through the interface 5 may comprise a material that has one or more of the following qualities: is robust to sharp objects; prevents adhesive layers or the like from puckering, or otherwise deforming, when it is applied to such adhesive layers; prevents or minimizes puckering of the enclosing outer layer, particularly when the interface 5 is deformed or bent; is impermeable to gas flow. The material may be a thermoplastic polymer, polyethylene terephthalate (PET), polyester film, biaxially-oriented polyethylene terephthalate (BoPET), or the like.

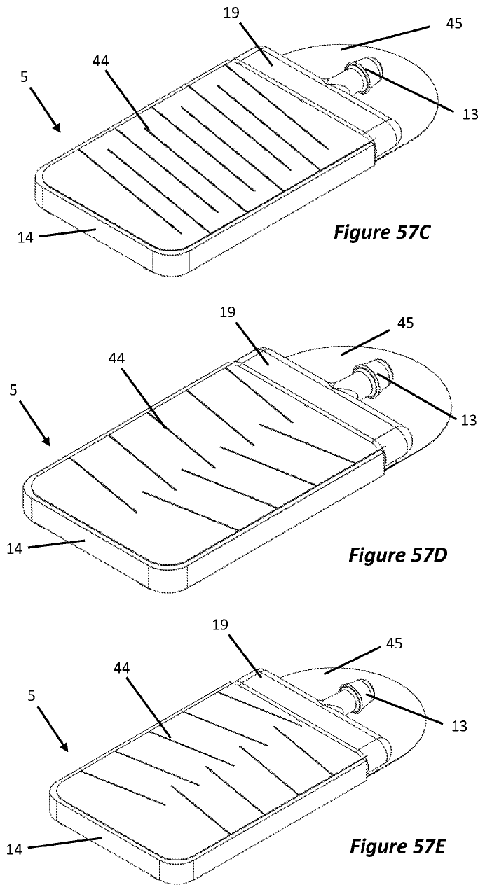

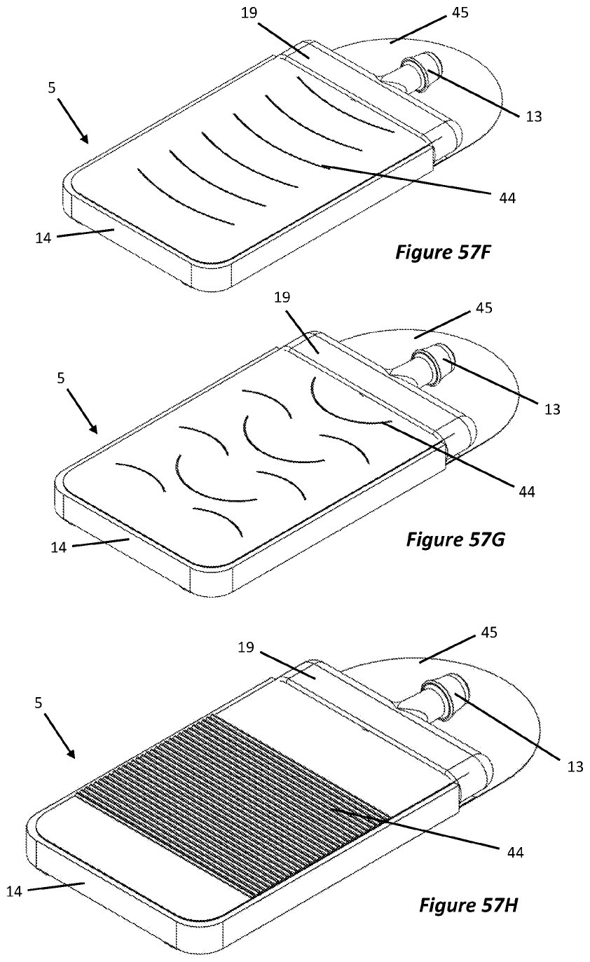

At least part of the enclosing or outer layer covering or defining at least part of the gas flow path of the interface 5 comprising the aforementioned material may have one or more creases, channels, grooves or slits 51 over at least part of a surface thereof to facilitate or impart flexibility of that layer and hence to the patient interface 5. These slits/grooves can be in any suitable arrangement configuration or pattern to impart flexibility, examples of which are shown in FIGS. 57A to 57H.

Referring to FIG. 57A, the grooves or slits may comprise a series of alternating slits/grooves 51 extending from alternating edges. The alternating slits/grooves 51 can be arranged in parallel with each other. The parallel slits/grooves 51 can be continuous, as in FIG. 57A, or discontinuous, such as in FIG. 57B. The alternating slits/grooves 51 can extend parallel to the outlet 14 or can be arranged at an angle relative to the sides and outlet 14, such as shown in FIG. 57C. A series of slits/grooves 51 may extend in from opposite sides such that first set of slits/grooves 51 extends from one edge and a second set of slits/grooves 51 extends from the other edge, the slits/grooves of one set being interleaved with the slits/grooves 51 of the other set.

A series of slits/grooves 51 may extend in from opposite sides. A first group of slits/grooves 51 can be oriented in a non-parallel angle with respect to a second group of slits/grooves 51. The slits/grooves 51 of each set may extend past a centreline or centre portion of the interface such that there is no linear path between inlet end and outlet end that is uncut by a slit/groove 51. The first and second groups of slits/grooves 51 may form a substantially herringbone pattern, an example of which is shown in FIG. 57D.

As shown in FIG. 57F, the slits/grooves 51 may be arranged in a pattern of successive slits/grooves 51 that arcuately extend toward a lengthwise edge of the interface 5. The slits/grooves 51 may be of uniform length and substantially uniformly spaced apart from each other.

The slits/grooves 51 may be arranged in columns of slits/grooves 51 that extend to opposing sides in alternation. Each of the columns of slits/grooves can comprise slits/grooves 51 with semicircular or crescent shapes that face the opposite direction to and are at least partially offset toward semicircular or crescent shapes of slits/grooves in an adjacent column, such as shown in FIG. 57G. In some embodiments, portions of the columns of slits/grooves 51 overlap in the longitudinal direction of the patient interface 5, i.e. in the longitudinal direction between inlet and outlet 14.

The slits/grooves 51 may include a series of parallel ridges and grooves, e.g. corrugations, such as shown in FIG. 57H. The corrugations may be located in one or more regions so as to encourage preferential bending of the patient interface 5 at one or more predetermined locations. In other words, the location of the corrugations can encourage bending in the region(s) of corrugation.

A gas-impermeable layer 41 may also at least partly coat or cover the support structure 19. The gas-impermeable layer 41 may prevent the flow of gasses such as air, carbon dioxide (CO₂), nitrogen gas (N₂), nitrogen dioxide N₂O, Argon (Ar), Helium (He) through the film. For example, the gas-impermeable layer 41 may block at least 95.0, 96.0, 97.0, 98.0, 99.0 or 99.9% of gas flow through the film. The gas-impermeable layer 41 may have a thickness that allow bending and/or deformation of the patient interface 5 when coated or layered by the gas-impermeable layer 41.

In each embodiment, a portion of the outer surface area of the gas permeable substrate 12 remains uncovered by gas-impermeable layer 41, thereby defining the interface outlet 14. The interface outlet 14 may be axially aligned with the interface inlet tube 13. Alternatively, the interface outlet 14 may be aligned perpendicularly or at an angle to the interface inlet tube 13. A portion of the surface area of the front face and/or side wall of the gas permeable substrate 12 may remain uncovered by the gas-impermeable layer 41. In one embodiment the gas permeable substrate 12 may be cylindrical having the gas-impermeable layer 41 coating or covering its surface wherein the interface outlet 14 is defined by one or more slits in the gas-impermeable layer 41. Such an embodiment may resemble the patient interface as shown in FIG. 4F. The diffused gas flow can flow out of the uncovered area of the gas permeable substrate 12. The gas-impermeable layer 41 may overlap or cover a portion of the support structure 19 which may affix the gas permeable substrate 12 to the support structure 19. The gas permeable substrate 12 may be adhered to the support structure 19 with adhesive. The gas-impermeable layer 41 defines a volume of gas permeable substrate 12. The gas-impermeable layer 41 may contain substantially all or most of the gas permeable substrate 12. A portion of the gas permeable substrate 12 may extend beyond the gas-impermeable layer 41, such as at or adjacent the interface outlet 14.

In some embodiments, one or more portions of the front face of the gas permeable substrate 12 is absent a covering of gas-impermeable layer 41. The patient interface 5 may in use, be located in or adjacent to the surgical cavity 6 with the rear face of the patient interface 5 adhered to a suitable surface, typically a surface of a medical instrument, such as a surgical retractor. The diffused gas flow fills the surgical cavity 6 as it flows out of the portion(s) of gas permeable substrate 12 of the front face not coated or layered in gas-impermeable layer 41.

A bottom side wall of the gas permeable substrate 12 may be absent the gas-impermeable layer 41 such that the interface outlet 14 is located opposite the support structure 19 and distal to the interface inlet tube 13. Examples of such an embodiment are shown in FIGS. 4E, 4G and 41. The patient interface 5 may in use be located in or adjacent to the surgical cavity 6 with the rear face of the patient interface 5 adhered to a suitable surface, such as surface of a surgical retractor. The diffused gas flow fills the surgical cavity 6 as it flows out of the interface outlet 14.

Alternatively, the patient interface 5 may be located in use at or adjacent the wound edge of the surgical cavity 6. Gas may fill the surgical cavity 6 as it flows out of the interface outlet 14, which may be located at a bottom side wall of the patient interface 5, such as the example shown in FIGS. 4E and 4G That is, the patient interface 5 may be adhered to the patient's skin or surgical drape adjacent the wound edge with the interface outlet 14 approximately aligned to the wound edge. The patient interface 5 may be positioned adjacent the wound edge so that the interface outlet 14 is directed at an elevated angle relative to horizontal or to the surface at the wound edge. For example, the interface outlet 14 may be directed at an angle 5, 10, 15, 20, 25 or 30 degrees from horizontal. A portion of gas permeable substrate 12 may extend outwardly from the support structure 19, such as is shown in FIG. 4E. This portion of gas permeable substrate 12 may be substantially less than its width. For example, referring to FIG. 4E, the gas permeable substrate 12 may extend the width of the support structure 19 and extend outwardly from the support structure 19 a length that is less than about a half, a third or a quarter of the width of the support structure 19. As discussed above, the gas permeable substrate 12 may abut the support structure 19, may be affixed at least partly over the support structure 19, or may fill a portion of the support structure 19. The support structure 19 may be formed at least partly of a soft and/or flexible material to allow the support structure 19 and hence the patient interface 5 to more readily conform to contours of the patient's body.

As discussed, the patient interface 5 may comprise an adhesive layer 42 on one side of the patient interface 5 as shown in FIGS. 4J and 4K. The adhesive layer 42 may be used to attach the patient interface 5 to the patient, retractor, surgical drape, surgical instrument or surgical equipment. The adhesive layer 42 may cover a portion of the rear face of the patient interface 5. For example, the adhesive layer 42 may cover a portion of the rear face of the patient interface 5 proximate to the interface inlet tube 13. The adhesive layer 42 may cover the upper half of the rear face of the patient interface.

The gas permeable substrate 12 and hence the patient interface 5 formed by the gas permeable substrate 12 may be flexible and/or deformable. In order to allow the patient interface 5 to be bent and to retain its shape, the patient interface 5 may comprise one or more deformable elements 40. The deformable element 40 may be in a variety of forms. For example, the deformable element 40 may be in the form of a wire within the patient interface 5. The wire may form a loop as shown in FIG. 4I. However, other configurations are possible, such as, but not limited to one or more deformable elements 40, members, wires or the like extending outwardly from the support structure 19. The deformable element 40 may also comprise one or more of a deformable plate or panel. The deformable element 40 may be continuous or may have features, such as cutaways, corrugations or pre-bends which may assist in controlling or preferentially defining how it can be deformed in use.

The deformable element 40 may be located within the gas permeable substrate 12, between the gas-impermeable layer 41/support structure 19 and the gas permeable substrate 12, within or on the outside of the gas-impermeable layer 41/support structure 19. In some embodiments the deformable element 40 may be coextruded with the gas-impermeable layer 41 and/or the support structure 19. The deformable element 40 allows the patient interface 5 to be bent to match or conform to geometry of the wound edge or surface onto which the interface is placed and to retain its shape. This allows the patient interface 5 to be located at the wound edge so that an upper portion of the patient interface 5 is located on the patient's skin or surgical drape.

The patient interface 5 can bend to so that the gas flow path defined by the walls is configured to provide a desired direction of gas flow. In other words, the patient interface 5 and its gas flow path can be configured to position the interface outlet 14 to provide a desired direction of gas flow. A desired direction of gas flow may be into the surgical cavity 6. For example, if the interface 5 is located on the wall of the surgical cavity and the interface outlet 14 is located on the lower surface of the interface 5, then the interface 5 can be bent to direct the gas flow horizontally from the interface outlet 14 if that is desired. If the patient interface 5 is positioned on the surface of the skin adjacent the surgical cavity 6, the patient interface 5 may be bent to position the gas flow path and/or outlet 14 such that gas is directed into the surgical cavity 6.

The adhesive layer 42 on the rear face of the patient interface 5 may be limited to the portion of the patient interface 5 that sits on patient's skin, surgical drape, or other suitable surface adjacent the surgical site/surgical cavity 6 (i.e. not against the patient wall of the surgical cavity).

The gas permeable substrate 12 may be removable from the support structure 19. That is, it may be desirable for the support structure 19 to be reusable and for the gas permeable substrate 12 portion of the patient interface 5 to be disposable. In such an embodiment the gas-impermeable layer 41 may not extend to cover the support structure 19 nor would the gas permeable substrate 12 be adhered with adhesive to the support structure 19. Instead, the gas permeable substrate 12 may be fixed to the support structure 19 by friction fitting a portion of the gas permeable substrate 12 within a portion of the support structure 19, through the use of removable tabs that may overlap the gas permeable substrate 12 and the support structure 19, or by any other suitable method of attachment.

Referring now to FIG. 4L, there is shown an embodiment of the patient interface 5 with a support structure 19 in the form of a connector 43. The connector 43 is provided adjacent to the gas permeable substrate 12. The connector 43 may have a substantially solid form, and comprise a passage there through to allow passage of the flow of gas from the interface inlet 47 or interface inlet tube 13 to an outlet of the connector 52. The connector 43 may be formed of a rigid material such as plastic, a flexible material such as silicon, or a combination of rigid and flexible materials.

The connector 43 may have a first surface 53 adjacent the interface inlet 47 and a second opposite surface 54 adjacent the gas permeable substrate 12. The connector 43 may comprise one or more passages through the connector from the interface inlet 47 or interface inlet tube 13 to the connector outlet(s) 52. The interface inlet 47 or interface inlet tube 13 may be on the first surface 53 of the connector 52. The connector outlet(s) 52 may be on the second surface 54 of the connector 43. The first surface 53 may be opposite the second surface 54. The gas flow from the connector outlet(s) 52 provide a flow of gas to the gas-permeable substrate 12.

The gas-permeable substrate 12 can abut to a side of the connector 43. In one embodiment the gas-permeable substrate 12 can abut or attach to a side of the connector 43. In the embodiment shown, the gas-permeable substrate 12 abuts or attaches to the second surface of the connector 43 opposite the inlet, so as to substantially cover the second end of the one or more apertures 44. As described above, the gas-permeable substrate 12 may be selected from any material and/or structure that is porous, for example, sintered plastics, open cell foams and other porous permeable substrates 18, for instance paper, felt, sintered metal and/or filter material.

At least a portion of the outer surface area of the gas permeable substrate 12 may be coated or layered in a gas-impermeable layer 41. The gas-impermeable layer 41 may also coat or cover at least a portion of the connector 43. In some embodiments the gas-impermeable layer 41 may also coat or cover a deformable element 40. The gas-impermeable layer 41 may help the gas permeable substrate 12 to remain abutted to the connector 43, without necessarily requiring any direct adhesion to be applied between the gas permeable substrate 12 and the connector 43.

In some embodiments, the gas-impermeable layer 41 may include one or more tabs 45 that extend over or about the connector 43. For example, as shown in FIG. 4L, the tabs 45 may extend about one or both sides of the interface inlet tube 13 where the interface inlet tube 13 joins to the connector 43.

The gas-impermeable layer 41 may define the interface outlet 14. That is, the interface outlet 14 is defined by the portions of the gas permeable substrate 12 not coated or covered by the gas-impermeable layer 41. The interface outlet 14 may locate at the opposite end of the patient interface 5 to the end of gas permeable substrate 12 that abuts the connector 43. The interface outlet 14 provides a flow of gas. The gas flow out of the outlet may be substantially unidirectional, bi-directional or omnidirectional.

In some embodiments the interface outlet may extend to the corners and/or sides of the gas permeable substrate 12. In some embodiments the gas flow out of the outlet may be omnidirectional. In some embodiments the gas flow out of the outlet may be omnidirectional in a single plane. The gas flow out of the outlet may be omni-directional in two or more planes. An omnidirectional flow may be achieved by having exposed gas permeable substrate 12 on two or more surfaces, where those surfaces are in a different plane relative to each other. For example, on a patient facing surface of the patient interface 5 and a side wall surface of the patient interface 5. The gas flow out of the outlet may be omni-directional in a single plane.

As shown in FIG. 4L the interface outlet 14 may locate on a surface that is opposite the wall that abuts the connector. The gas-impermeable layer 41 may include one or more slots or cutouts 56 to define or extend one or more interface outlets 14 from the gas permeable substrate 12. The one or more slots or cutouts may be located on the front, rear and/or side face(s) of the gas permeable substrate 12. As defined herein the “rear face” of the gas permeable substrate 12 is the patient facing side and the “front face” is the opposite face to the rear face.

As shown in FIG. 4L the impermeable layer 41 has a cutout that exposes a portion of the rear face of the gas permeable substrate 12. This allows diffuse gas flow to exit from the gas-permeable substrate 12 via the cutout, which therefore defines at least a part of the interface outlet 14. The cutout may also function to mitigate or avoid possible occlusion or obstruction of the interface outlet 14 by reducing risk of opposing adhesive layers sticking to each other at or adjacent the outlet 14.

The gas permeable substrate 12 may be flexible and/or deformable. The patient interface 5 may, as mentioned above, comprise one or more deformable elements 40 to allow the interface 5 to be bent as required and to retain its shape once bent. The, or each, deformable element 40 can be located in or adjacent the gas-permeable substrate 12.

In the embodiment shown in FIG. 4L, the patient interface 5 includes a single deformable element 40. The deformable element 40 may be located between the gas-impermeable layer 41, and the gas permeable substrate 12. That is, the deformable element 40 may be located external to the gas permeable substrate 12.

The deformable element 40 may be attached to the connector 43. The deformable element 40 may be fixed or otherwise attached to the second surface of the connector 43. An end or ends of the deformable element 40 may be fixed substantially at or adjacent one side of the second surface of the connector 43. As shown in FIG. 4L, respective ends of the deformable element 40 are attached to and extend outwardly from the second surface in a spaced apart configuration adjacent an upper or non-patient facing side of the connector 43. Ends of the or each deformable element 40 may alternatively be attached to and extend outwardly from the second surface in a spaced apart configuration adjacent the patient facing side of the connector 43. Thus, when the gas-permeable substrate 12 is abutted or attached to the connector 43, the deformable element 40 may locate on a face of the gas-permeable substrate 12.

The deformable element 40 allows the patient interface 5 to be bent as required. For example, the patient interface 5 can be bent or deformed to match or conform to geometry of the surface, such as wound edge or retractor and to retain its shape. This allows the patient interface 5 to be positioned at or adjacent the wound edge or on the surface as required. The patient interface 5 can bend to allow the outlet 14 to be directed generally downwards towards the surgical cavity 6. This can assist the process of filling the surgical cavity 6 with the gas flow directed outwardly from the interface outlet 14.

At least one adhesive layer 42 may be applied to one or more of the faces of the gas-permeable substrate 12. The at least one adhesive layer 42 may be double-sided. In some embodiments the gas-impermeable layer 41 may partially cover the gas permeable substrate 12. Subsequent layers, such as an adhesive layer 42 may thereby attach to the gas permeable layer 41 and the gas permeable substrate 12.

As shown in FIG. 4L the gas-impermeable layer 41 covers a substantial portion of the rear face of the gas-permeable substrate 12 and may extend to the side walls of the gas-permeable substrate 12 and a part thereof of the front face of the gas-permeable substrate 12. FIG. 4L shows one embodiment that includes two separate adhesive layers 42. An adhesive layer 42 may attach to the gas-impermeable layer 41 on the rear face and the gas-impermeable layer 41 and gas-permeable substrate 12 on the front face.

As stated, the at least one adhesive layer 42 may be applied to the face of the gas-permeable substrate 12 that already has the gas-impermeable layer 41 applied. In such an instance, the at least one adhesive layer 42 may be formed to be of a shape, and a size, so that when applied to the face of the gas-permeable substrate 12 it does not extend beyond the area that is covered by the gas-impermeable layer 41.

The patient interface 5 may include a layer, such as a protective backing 48 shown in FIG. 4L, which can be removed to expose the adhesive layer 42. The protective backing may be formed from non-adhesive, substantially robust, material. The adhesive layer 42 may coat the rear face of the patient interface 5, or a portion thereof.

With reference to FIG. 4L, the patient interface 5 may comprise a separate adhesive layer. The patient interface 5 may also comprise a top layer 50 that has a thickness that confers protective properties. The thicker layer 50 may also extend to the side walls of the patient interface 5. The thicker layer 50 may be applied, such as by the use of adhesive. The thicker layer 50 may have adhesive coated on one of its faces.

The at least one adhesive layer 42 may have an external film 50 applied to it. The external film 51 may be composed of a material that has at least the following properties: is robust to sharp objects; prevents adhesive layers or the like from puckering, or otherwise deforming, when it is applied to such adhesive layers; is impermeable to gas flow.

In some embodiments, any of the layers about the gas permeable substrate 12 may include one or more creases, channels, grooves or slits 51 over at least part of a surface thereof to facilitate or impart flexibility of that layer and hence to the patient interface 5. These slits/grooves 51 can be in any suitable arrangement configuration or pattern to impart flexibility.

The patient interface 5 may be positioned in the surgical cavity 6 against the patient wall or a medical instrument such as surgical retractor. The diffused gas flow thus fills the surgical cavity 6 as it flows out of the gas permeable substrate 12 of the patient interface 5. The adhesive layer 42 may be used to attach the patient interface 5 to the patient, retractor, surgical drape, surgical instrument or surgical equipment.

Gas may fill the surgical cavity 6 as it flows out of the interface outlet 14. The patient interface 5 may be adhered to the patient's skin or surgical drape adjacent the wound edge with the interface outlet 14 approximately aligned to the wound edge. The patient interface 5 may be positioned adjacent the wound edge so that the interface outlet 14 is directed at an angle relative to horizontal or to the surface at the wound edge. For example, the interface outlet 14 may be directed at an angle 5, 10, 15, 20, 25 or 30 degrees from horizontal.

FIG. 5A shows a patient interface 5 in the form of a gas-permeable substrate 12. A gas-permeable substrate 12 is efficient at slowing gases in a confined volume of space. The gas-permeable substrate 12 may not be solely dependent on the geometry of the patient interface 5 and can be adapted to suit most geometries.

The gas-permeable substrate 12 may sit at the outlet of the delivery assembly 4, for example within the outlet 14 of the patient interface 5. The gas-permeable substrate 12 may comprise pores that create a labyrinth of gas flow pathways which spread flow out and engage a larger outlet area consequently slowing the exiting gas's velocity. The pores of the gas-permeable substrate 12 may generate a network of passages for the gas to distribute across.

The porosity of the gas-permeable substrate 12 may be altered to influence the resistance to flow. For a given flow rate, increasing the resistance to flow increases the required driving pressure of the gas supply. For a given flow rate, as the resistance to flow is decreased, this decreases the required driving pressure of the gas supply. The alteration of the resistance to flow may also alter the degree of gas distribution throughout the porous medium and consequently change the reduction in gas velocity.

The porous medium may be selected from soft, resilient and/or flexible materials. Preferably the porous medium is selected from woven fabric, felt, porous films, woven mesh or fibrous materials, such as wool or soft filters. In one embodiment the porous medium is selected from hard or rigid materials. In some embodiments, the porous medium is selected from sintered metal, sintered polymer/plastic or sintered ceramic. In one embodiment the porous medium is selected from granular substrates. Preferably porous medium is selected from one or more of sand, carbon, garnet, anthracite or other suitable particulate material

FIG. 5B demonstrates the flow of gas through the gas-permeable substrate 12, showing that the gas flow follows an expanding path through the gas-permeable substrate 12 between the input to the gas-permeable substrate 12 and the outlet from the gas-permeable substrate 12. In one embodiment the patient interface 5 may comprise a porous medium 12 that sits at a point where the cross-sectional area of the patient interface 5 transitions from small to large. That is, the interface outlet 14 is expanded relative to the interface inlet.

The patient interface 5 may have a cross sectional area that expands along its length as shown in FIGS. 6A and 6B. FIG. 6A shows a flow path where a relatively high velocity flows from an inlet with small cross-sectional area and evenly distributes it across an outlet (or outlets) with a higher cross-sectional area. FIG. 6 shows a cross section of an expanding outlet, that is, the flow path expands in such a way that the flow remains attached and laminar. Without wishing to be bound by theory, this design reduces flow separation and allows the gas to slow down as the static pressure of the gas is recovered. In one embodiment this may involve a smooth transition between the inlet and outlet over a long gas path. In one configuration the patient interface does not comprise a gas-permeable substrate 12.

FIG. 6B shows a further embodiment of a patient interface 5 that conditions gas flow at least in part by having a cross sectional area that expands along its length from inlet 13 to outlet 14. In this embodiment, the patient interface 5 is configured to deliver or emit a flow of substantially laminar or non-turbulent gas flow. The gas flow emitted or delivered outwardly from the outlet 14 may be a single direction gas flow. The patient interface 5 of FIG. 6A is similar to the embodiment shown in FIG. 6 in that it has an inlet, outlet 14 and a chamber 55 defining a volume.

The inlet has a smaller cross-sectional area relative to the outlet 14. The chamber 55 widens laterally outwardly from a narrow neck portion adjacent the inlet to a wide portion. The chamber 55 has a bottom or patient-facing surface or wall and an opposing upper wall. The bottom and/or upper surface may each be substantially planar. One or both bottom and upper surface may be generally inwardly or outwardly curving. The inwardly or outwardly curving of the upper and/or bottom surfaces may be generally localised at the wide portion of the chamber 55. In the embodiment shown, the upper surface is generally convex and the patient facing is generally concave. A generally concave lower surface can be configured to match the general shape of the surface to which the patient interface 5 is intended to be applied or placed.

The chamber 55 may have one or more internal structures located or configured to guide gas flow as it enters the chamber 55. The one or more internal structures may comprise flow guide elements, such as baffles. The internal structures are located in the chamber 55 and configured to distribute/direct gas flow from the inlet substantially across the cross-sectional area of the outlet 14.

Gas flow can be additionally conditioned by gas-permeable substrate 12 located in the chamber 55. In the embodiment shown, the gas-permeable substrate 12 is located at or adjacent the outlet 14. The gas-permeable substrate 12 may span the entirety of the outlet 14 and extend a distance inwardly from the outlet. In the embodiment shown, the gas-permeable substrate 12 substantially fills the widest portion of the chamber/patient interface. Alternatively, the gas-permeable substrate may fill a larger volume of the chamber. As per other described embodiments, the gas permeable substrate 12 conditions the gas flow and in the context of this embodiment, conditions the gas flow such that gas exiting the outlet 14 is directed outwardly substantially in one direction or plane and/or as a laminar or non-turbulent flow. The one or more internal structures distribute or direct gas flow from the inlet substantially across the surface of the gas-permeable substrate 12 located at or adjacent the outlet 14. Gas entering the chamber through the inlet can be directed towards the gas-permeable substrate 12 directly from the inlet 13 as well as laterally/outwardly as directed by the internal structures.

The patient interface 5 may additionally comprise a deformable or shape conforming element or component. The deformable element may be substantially as described with reference to the embodiment of FIGS. 4I-4L. The deformable or shape conforming element may alternatively or additionally comprise a deformable component in contact with the lower surface of the chamber. The deformable component may comprise a deformable pad or cushion. The deformable pad or cushion may be comprised of a suitable material, such as foam. The foam may be a memory foam or similar. The deformable pad or cushion can be deformed as appropriate to conform to the surface onto which the patient interface 5 is placed or attached.

In one embodiment different gas pathway geometries are tuned for specific flowrate ranges. Furthermore, the rate of expansion along the length of the gas pathway of the cross-sectional area may be more gradual for higher flows (e.g. 20 L/m) compared to lower flows (e.g. 5 L/min).

In one embodiment the interface produces a gas flow that comprises incident flow. For example, the gas flow exiting into the surgical cavity 6 is a result of colliding flow leading to a gas flow into the surgical cavity 6 that is dispersed and spread. In one embodiment the gas flow is directed to a surface as shown in FIG. 7A. The flow collides with the surface and then spreads laterally. The lateral flow is therefore incident flow from the main flow that has collided with a surface. While a flat linear surface is shown in FIG. 7A, it will be appreciated that a collision surface with a range of different geometries can be used. For example, a convex surface will reflect the collided gas in a range of angles, including compared to a flat surface as shown in FIG. 7A. The nature of the surface may also be varied. For example, a smooth surface may reflect the air much more directly than a rough surface that will produce a flow with its velocity attenuated. The surface roughness can be modified appropriately to achieve the desired result.

The gas flow into the surgical cavity 6 may result from two or more gas flows colliding as shown in FIGS. 7B and 7C. That is, the flow from the gas source is delivered along two or more tubes with their outlet directed substantially toward each other. The two or more tubes comprising the outlet ends of the patient interface 5 may split off from a main tube. The main tube, and the tube comprising the outlet ends of the patient interface 5 may all form part of the patient interface 5 with an inlet end at the main tube for connection to the delivery assembly 4.

In one embodiment the collided gas flow may result from the use of internal baffles within the patient interface 5 as shown in FIG. 7D. As shown by the arrows that indicate the direction of gas flow, the gas hits the opposed wall of the base of the patient interface 5 and then exits via the interface outlet 14 in a direction substantially orthogonal to the initial direction of the gas flow. The collided gas flow produces a diffuse flow of gas into the surgical cavity 6 with a decreased velocity.

The two or more outlets of the patient interface 5 may be located outside of the surgical cavity 6 on opposed surfaces. As shown in FIG. 7C, the gas flow jets out of the respective outlets and the jets collide together over the surgical cavity 6. In one embodiment the collision of gas flow produces a turbulent area above the surgical cavity 6. The turbulent site may assist in repelling airborne contaminants from entering the surgical cavity 6, such as by creating a protective layer or ‘curtain’ of gas.

In the above example, the effective outlet area of the patient interface 5 is notionally the area of turbulence where the two or more jets of gas flow meet.

Due to the collided nature of the gas flow at the interface, the gas flow has an outlet surface area increased compared to the flow of gas exiting the interface prior to the collision. That is, the vector or direction of flow is much more diverse and includes a radial flow component from the point of collision. Additionally, the velocity of the gas flow is reduced at the interface outlet.

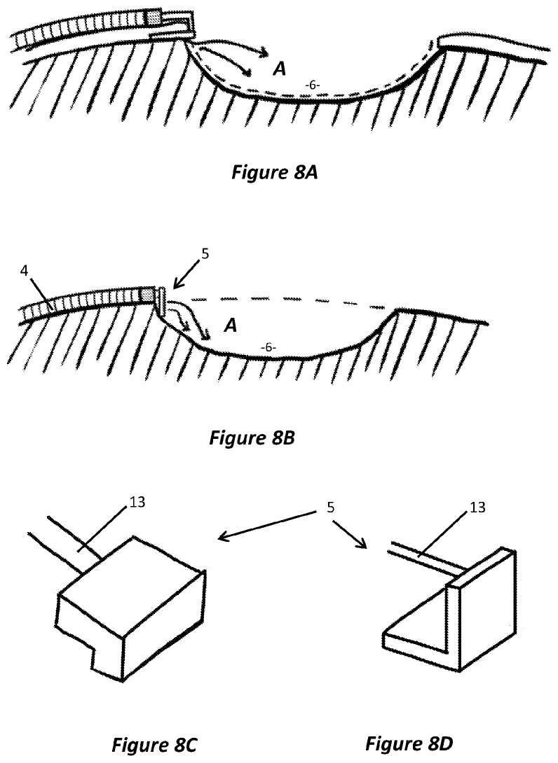

As shown in FIGS. 8A to 8C, the patient interface 5 may be substantially L-shaped or substantially U-shaped. The patient interface 5 may be positioned close to the wound edge and may also extend to the upper patient wall of the surgical cavity 6 to flood the surgical cavity 6 with gas from the top down.

In some embodiments the substantially L-shaped or U-shaped interfaces are made from soft or compliant materials. In an alternate embodiment the substantially L-shaped or U-shaped interfaces are made from resilient or rigid materials.

In one embodiment as shown in FIG. 8A, the patient interface 5 is substantially U-shaped. At least a portion of the patient interface 5 (i.e. one of the arms of the “U”) can hook under the tissue layer of the wound edge as shown in FIG. 8A. As shown, the gas flow from the patient interface conduit 11 of the delivery assembly 4 enters one of the arms of the patient interface 5 and is directed downwards into the surgical cavity 6. The interface outlet 14 of the patient interface 5 can be positioned at the base of the “U” to provide for the gas flow into the surgical cavity 6. For example, the interface outlet 14 of the patient interface 5 may be located on the arm that hooks under the tissue layer, proximal to the vertical portion of the U-shaped patient interface 5, with the distal portion of the arm extending to hook under the tissue layer to retain the patient interface 5 in place. Alternatively, the interface outlet 14 may be located on the vertical portion of the “U” and may connect to the arm that hooks under the tissue layer. That is, the arm that hooks under the tissue layer may or may not be integrally formed with the remainder of the U-shaped patient interface 5. The interface outlet 14 of the patient interface 5 may be in a variety of forms. For example, it may comprise a plurality of apertures, a porous medium or any of the aforementioned mechanisms for providing a conditioned flow of gas from the interface outlet 14. In some embodiments the interface outlet 14 may be located on any surface of the U or L shape. For example, with reference to FIG. 8C the interface outlet 14 may be on the lower surface, or on the vertical surface of the “L”. With reference to FIG. 8D the interface outlet 14 may be on the vertical surface of the “L”.

In one embodiment the patient interface 5 is substantially L-shaped. An example is shown in FIGS. 8B and 8C. An arm of the L connects to the delivery assembly 4, the connection optionally being above or adjacent the surgical cavity 6. The orthogonal arm of the L shape then extends downwardly into the surgical cavity 6. The downwardly extending portion of the patient interface 5 may contain the interface outlet 14. In some embodiments the interface outlet 14 is located on the lower portion of the downwardly extending portion of the patient interface 5. In some embodiments, as shown in FIG. 8C the interface outlet 14 is located on a downward portion of the patient interface 5, i.e. the lower portion of the patient interface 5 that directs air downwardly into the surgical cavity 6. The interface outlet 14 of the patient interface 5 may be in a variety of forms. For example, it may comprise a plurality of apertures, comprise a porous medium, or any of the aforementioned mechanisms for providing a conditioned flow of gas from the interface outlet 14.

In one embodiment the patient interface 5 is substantially L-shaped. An example is shown in FIG. 8D, where a downwardly oriented arm of the “L” connects to the delivery assembly 4 either directly or via an interface inlet tube 13. The orthogonal arm of the “L” may hook under the tissue layer of the wound edge to retain the patient interface 5 in place. The interface outlet 14 of FIG. 8D is located on a downward facing portion of the patient interface 5, (i.e. the lower portion of the patient interface 5 that directs air downwardly into the surgical cavity 6). The interface outlet 14 could be located on any surface of the “L” shaped patient interface 5. For example, the outlet may be located at the join of the two arms of the “L”, or on the face of the downward portion (i.e. the vertical surface) of the patient interface 5. The interface outlet 14 of the patient interface 5 may be in a variety of forms. For example, it may comprise a plurality of apertures, comprise a porous medium, any of the aforementioned mechanisms for providing a conditioned flow of gas from the interface outlet 14.

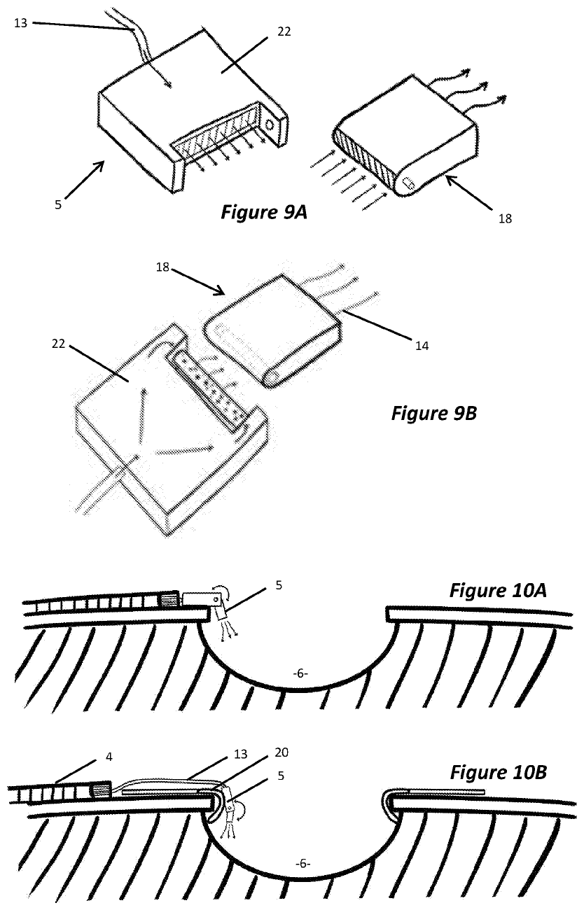

FIGS. 9A and 9B each show an exploded view of a patient interface 5 comprising a hinged or articulated portion 18, wherein the patient interface outlet 14 is located on the hinged or articulated portion 18. The patient interface 5 may therefore comprise a first portion 22 comprising the interface inlet tube 13 or connection to the delivery assembly 4, and a connection for the hinged or articulated portion 18. The patient interface 5 may comprise a gas-permeable substrate 12 in either of the first portion 22 or the hinged or articulated portion 18 of the patient interface 5, or in both portions. The gas-permeable substrate 12 may partly fill the cavity of the first portion 22 and/or the hinged or articulated portion 18. In one embodiment the gas-permeable substrate 12 may fill the cavity of the first portion 22, the hinged or articulated portion 18 or both the first portion 2 the hinged or articulated portion 18 of the patient interface 5.

With reference to FIG. 9A, the hinged or articulated portion 18 is connected to the first portion 22 via a pair of pins and corresponding recesses. Each pin is rotatably located in a corresponding recess, enabling the hinged or articulated portion 18 to move relative to the first portion 22. Gas flow (shown by the arrows) enters the first portion 22 via the interface inlet tube 13 and out of the first portion 22 at an interface with the hinged or articulated portion 18. Gas flow continues through the hinged or articulated portion 18 and out of the patient interface outlet 14. In this configuration, gas can flow outwards across substantially all of the surface area of the patient interface outlet 14. The interface between the first portion 22 and the hinged or articulated portion 18 may comprise gas-permeable substrate 12 at the first portion 22, the hinged or articulated portion 18, or both the first portion 22 and the hinged or articulated portion 18. The hinged or articulated portion 18 may comprise an inlet (that interfaces with the outlet from the first portion 22) that extends the width of the hinged or articulated portion 18. The inlet to the hinged or articulated portion 18 may extend along the edge of the hinged or articulated portion 18. The inlet to the hinged or articulated portion 18 may extend from the edge of the hinged or articulated portion 18 to the lower facing of the hinged or articulated portion 18 such that when the hinged or articulated portion 18 is rotated about the pin, the inlet remains interfaced with the outlet of the first portion 22.

Referring now to FIG. 9B, there is shown another configuration of a patient interface 5 with hinged or articulated portion 18. In this configuration, the connection between first 22 and hinged 18 portions may be a cylinder located at or adjacent the interface between the first portion 22 and hinged or articulated portion 18. The cylinder may have opposing open ends and a plurality of apertures over a surface thereof. Gas flow (shown by the arrows) may flow into the first portion 22 from the interface inlet tube 13 and into the cylinder via the open ends. The gas flow may then flow through the apertures of the cylinder to enter the hinged or articulated portion 18. Gas may then flow outwardly from the patient interface outlet 14, with gas flow distributed substantially evenly across the surface area of the patient interface outlet 14.

As shown in FIGS. 10A and 10B the patient interface 5 having a hinged or articulated portion 18 can be positioned close to the wound edge or upper patient wall of the surgical cavity 6. For example, as shown in FIG. 10A the first portion 22 of the patient interface 5 can be positioned close to the wound edge such that the hinged or articulated portion 18 of the patient interface 5 can be rotated to direct gas flow into the surgical cavity 6. As shown in FIG. 10A, the patient interface 5 may be positioned close to the wound edge to extend to the upper patient wall of the surgical cavity 6. The patient interface 5 may be connected to a retractor 20, the retractor being used to maintain the wound edges apart to allow access to the surgical cavity 6. The hinged or articulated portion 18 of the patient interface 5 may be rotated to direct gas flow into or across the surgical cavity 6.

The hinged or articulated portion 18 of the patient interface 5 can be oriented as desired, at the start of and during surgery to direct gas flow into the surgical cavity 6.

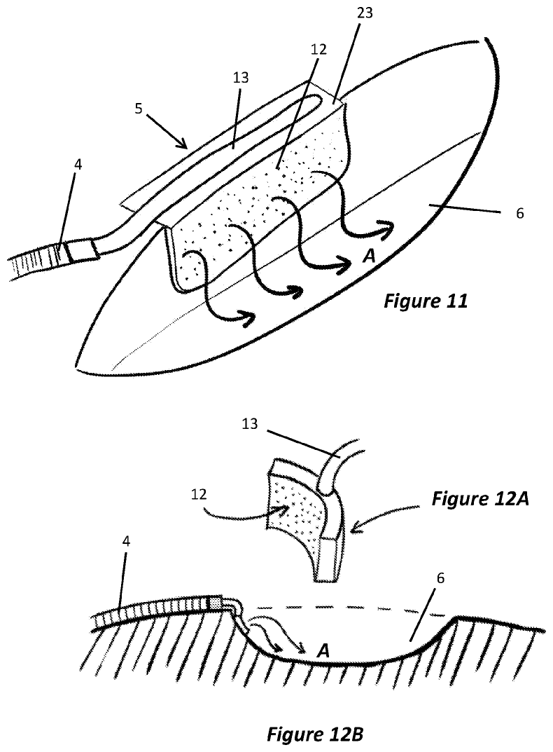

In one embodiment the patient interface 5 comprises a flexible pad 23 comprising a gas outlet zone having one or more gas flow paths for delivering gas into the surgical cavity 6. The patient interface 5 comprises an interface tube 13 for connecting to the delivery assembly 4. The patient interface 5 may sit on, adjacent or about the surgical cavity 6. For example, in one embodiment as shown in FIG. 11, the patient interface 5 sits at the wound edge of the surgical cavity 6, with the flexible pad extending downwardly into the surgical cavity 6. In one embodiment, at least a portion of the patient interface 5 extends into the surgical cavity 6. The portion extending into the surgical cavity 5 may comprise the interface outlet 14. The outlet of the patient interface 5 may be in a variety of forms. For example, it may comprise a plurality of apertures, or comprise a porous medium. As the patient interface 5 includes a flexible pad, it may be rolled, folded or otherwise manipulated or moved out of the way as required. For example, the portion of the patient interface 5 that extends into the surgical cavity 6 may be folded up out of the surgical cavity 6, such as when access is required as part of the medical procedure.

In one embodiment the patient interface 5 comprises an adhesive surface for attachment to the surgical area. The adhesive surface may be used for other forms of the patient interface 5 described herein, and may be used for other components of the example medical gases delivery system 1, such as the delivery assembly 4, or the interface inlet tube 13. For example, the patient interface 5 may attach to a surgical gown and/or drape, the patient's skin or surgical equipment. The adhesive may be under a peel off layer. That is, a layer is peeled off a bottom surface of the flexible pad to expose the adhesive surface. The adhesive zone may be located on any part of the flexible pad. For example, it may be located on a portion thereof of the patient interface 5 that sits outside of the surgical cavity 6, or attaches to the gown, patient's skin or retractor. The adhesive zone may be located on the reverse side of the outlet portion of the patient interface 5. The adhesive zone may be located in both the areas: the portion of the patient interface 5 that sits outside of the surgical cavity 6 and the reverse side of the outlet portion of the patient interface 5.

The patient interface 5 may comprise an interface inlet tube 13 providing a gas flow inlet that is distributed the length of the patient interface 5. The interface inlet tube 13 may have a plurality of apertures, slits or other similar openings substantially along its length. This may assist in providing an even outlet flow of gas from the patient interface 5 along its length. The delivery assembly 4 may attach directly to the patient interface 5 and may attach at one end as shown in FIG. 11, or may attach at any position along the length of the patient interface 5.

The patient interface 5 may have a slim profile (i.e. thin), to assist in keeping the patient interface 5 as unobtrusive as possible.

The patient interface 5 may also connect to a surgical retraction system or surgical retractor 20. A surgical retractor system or surgical retractor 20 is a surgical instrument used to separate the edges of a surgical incision or wound and/or to hold back underlying organs and tissues so that body parts under the incision may be accessed. This is described below. The flexible pad may wrap around, envelop or adhere to the retractor 20. This allows the flexible pad to be retained in place.

In one embodiment the flexible pad 23 may comprise a gas outlet surface and may have an adhesive surface on an opposed side of the pad to the outlet. An example of this is shown in FIG. 12A. The patient interface 5 may comprise a very thin and highly flexible porous film with an adhesive backing that can attach to any surface, such as the wound edge, or within the surgical cavity 6 as shown in FIG. 12B.

The flexible pad 23 may be made from a flexible film having a porous membrane. In one embodiment the flexible pad 23 may be formed of a material which may retain to a manipulated shape.

The flexible pad 23 may comprise an interface inlet tube 13 that delivers the gas flow to the flexible pad 23. The interface inlet tube 13 may be integral with the flexible pad 23 or may attach to the flexible pad 23. In one embodiment the interface inlet tube 13 is formed unitarily with the flexible pad 23 and connects to the delivery assembly 4 at a point spaced from the flexible pad. The interface inlet tube 13 may form an inlet to the flexible pad 23 at one end of the pad, or a side wall of the pad.

As shown in FIG. 12B the flexible pad 23 may sit within the surgical cavity 6, adhering or lying adjacent to a wall of the surgical cavity 6. The flexible pad 23 provides a conditioned gas flow into the surgical cavity 6.

The flexible pad 23 may connect to a retraction system, such as but not limited to one or more ring retractors. The flexible pad 23 may adhere to a portion of the retractor. This allows the flexible pad 23 to be retained in place.

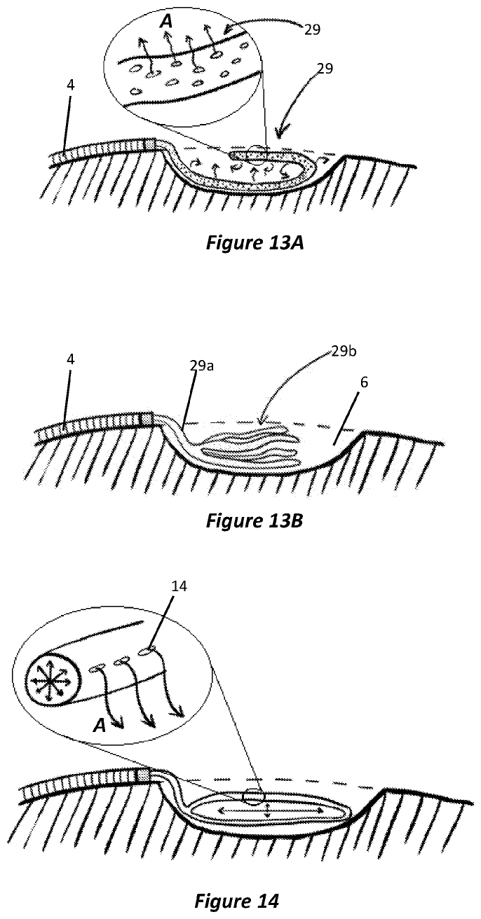

In one embodiment the patient interface 5 comprises one or more elongate body(s) 29 having a substantially tubular profile. An example of this can be seen in FIG. 13A. The elongate bodies 29 of the patient interface 5 may be formed from resilient or soft material, such as, but not limited to silicone or malleable metal. The elongate bodies 29 may be manipulated into a desired shape and retain to that desired shape.

The elongate bodies 29 may be formed of a material that retains to a desired shape. For example, the elongate bodies 29 may comprise soft material comprising a wire along its length. The wire may have a low elastic deformation limit and high fatigue life. A non-limiting example may be armature wire. As the elongate bodies 29 are manipulated into a desired shape, the wire retains the elongate body in that shape.

The elongate bodies 29 may be formed of a material that is resilient (i.e. moves back into shape after bending). The material may be selected from a soft plastic or silicon, or a spring steel.

The elongate body 29 may include one or more secondary elongate bodies 29b that stem off from a primary elongate body 29a. An example of this can be seen in FIG. 13B.

The interface outlet 14 or outlets are located on at least a portion of the primary elongate body 29a and/or secondary elongate bodies 29. As shown in FIG. 13A the interface outlet 14 may be formed of a plurality of small apertures that cover a portion of an elongate body. For example, the apertures may be located on the primary elongate body 29a, a secondary elongate body 29 or both the primary elongate body 29a and a secondary elongate body 29.

One or more of the elongate bodies 29 may sit within the surgical cavity 6. The elongate bodies 29 may be flexible, which facilitates their locating into the surgical cavity 6 as the elongate bodies 29 can be manipulated into convenient positions, particularly around any internal geometry.

In one embodiment the walls of the elongate bodies 29 are formed from a porous medium. In such a design the elongate bodies 29 comprise a tube for transporting the gas flow, the walls of the tube being formed of a porous medium configured to condition the gas flow.

In one embodiment an elongate body 29, or one or more of the elongate bodies 29, may form a loop as shown in FIG. 14. Again, the interface outlet 14 may be located on any portion of the body and may comprise a plurality of apertures or a wall formed from a porous medium.

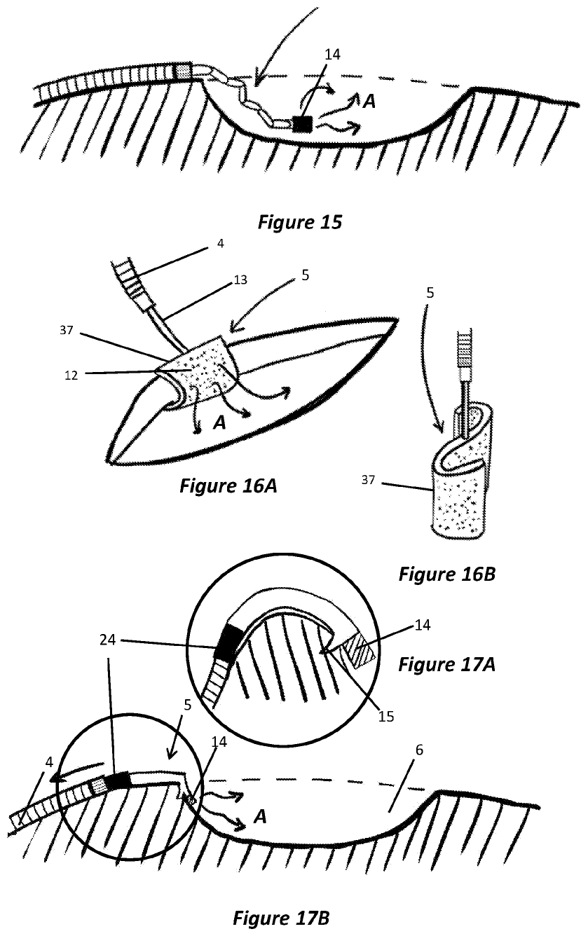

In one embodiment the patient interface 5 is connected to a tube that has one or more articulations. For example, FIG. 15 shows a patient interface 5 in which the interface outlet 14 is connected to the end of an articulating tube. The articulated tube can be deformed, oriented or otherwise manipulated around the surgical environment while still retaining its shape. The interface outlet 14 can be positioned inside the surgical cavity 6 in a way that suits the surgeon, and then be left to hold its position. The articulated tube may be formed as a series of ball/socket connections, corrugations, pliable material selection, or combination thereof, such as a combination of a deformable rod in a flexible tube.

The articulated tube may form part of the patient interface 5 and may be formed unitarily with the patient interface 5. In this embodiment the articulated tube has an inlet that connects to the delivery assembly 4. The connection may be attached with a screw thread or friction fit connection. The connection point between the articulated tube and the delivery assembly 4 may be spaced from the surgical cavity 6. Such a design may assist in keeping bodily fluids off the delivery assembly 4, which may be a reusable item and so protection from bodily fluids may be useful to ensure it can be reused.

In an alternative embodiment the articulated tube may form part of the delivery assembly 4. The outlet of the articulated tube will then interface with the patient interface 5 that attaches to the end of the articulated tube.

In one embodiment the articulated tube is formed from a soft and/or flexible material such that the articulated tube can be manipulated to form any shape but does not maintain that shape.

The patient interface 5 may be, or comprise, a flexible pad 37. An example is shown in FIGS. 16A and 16B. The flexible pad 37 may be formed of a material that retains its shape when subjected to plastic deformation and can be deformed in both the longitudinal and lateral directions. The flexibility of the pad allows it to be located into various parts of the surgical cavity 6 such as between organs, or under the wall of the surgical cavity.

The interface outlet 14 for the patient interface 5 is located on the flexible pad 37. In some embodiments the flexible pad 37 may be formed from a porous medium. The flexible pad 37 may include an interface inlet tube 13 formed unitarily with the flexible pad 37. In this embodiment the interface inlet tube 13 has an inlet that connects to the delivery assembly 4.

In an alternative embodiment the interface inlet tube 13 may form part of the delivery assembly 4. The outlet of the articulated tube will then interface with the flexible pad 37 that attaches to the end of the articulated tube. The articulated tube may connect to a face of the flexible pad 37, or an end of the pad.

When in use the flexible pad 37 may be cut down to a convenient size and then used in the surgical cavity 6. For example, the flexible pad 37 may be cut to a size to fit the physical dimensions of the cavity or to fit around obstructions in the surgical cavity 6 (e.g. surgical instruments, organs, physiological projections). This allows a single flexible pad 37 to be utilised in a variety of different surgical cavities having different sizes, or being of different types of cavities.

In one embodiment the patient interface 5 comprises a body having an interface outlet 14 at one end and an interface inlet tube 13 for connection to the delivery assembly 4 at the other end.

An example of the interface is shown in FIGS. 17A and 17B. The body of the patient interface 5 includes one or more projections 15 adapted to penetrate the tissue layer of the patient to assist retention of the patient interface 5 in place. As shown in FIG. 17A the projection 15 may be located at the outlet end of the patient interface 5, such that retention of the patient interface 5 is carried out by penetration of the projection 15 into the surgical cavity 6, as shown in FIGS. 17A and 17B. The penetration of the projection 15 for retention may be below the wound edge or into the upper patient wall of the surgical cavity 6. Such a design is useful where the patient interface 5 sits about the wound edge or extends between the wound edge and the upper patient wall of the surgical cavity 6. The projection 15 may penetrate the skin or tissue of the patient at a location spaced apart from the surgical cavity 6 with the interface outlet 14 of the patient interface 5 sitting within the surgical cavity 6 as shown in FIG. 17A.

In an alternative embodiment the projection 15 may be located distal from the interface outlet 14 of the patient interface 5, such that retention of the patient interface 5 is carried out by penetration of the projection at the upper patient wall of the surgical cavity 6.

In one embodiment the interface outlet 14 of the patient interface 5 comprises a zone of greater weight and/or density 24. This zone may be provided by employing a material with greater density than the material used to form the body of the patient interface 5. For example, the zone may be formed by a metal or a solid plastic. In one embodiment the zone of greater weight and/or density 24 is provided for by an attachment. The attachment can attach to the body of the patient interface 5 at any point along its length. The attachment may be a sleeve that forms about the body of the patient interface 5 or it may lock into the body of the patient interface 5.

The zone of greater weight 24 may combine with the projection 15 to provide a patient interface 5 that may be held against the upper patient wall of the surgical cavity 6. In one embodiment the combination of the projection 15 and the weighted end of the patient interface 5 provides for a substantially self-locking mechanism once the projection 15 penetrates the tissue layer at the wound edge or the upper patient wall of the surgical cavity 6, particularly when the projection 15 is provided in a hook-like configuration, as shown in FIG. 17A. The body of the patient interface 5 may be formed as an arc to facilitate passage across the upper edge of the surgical cavity 6.

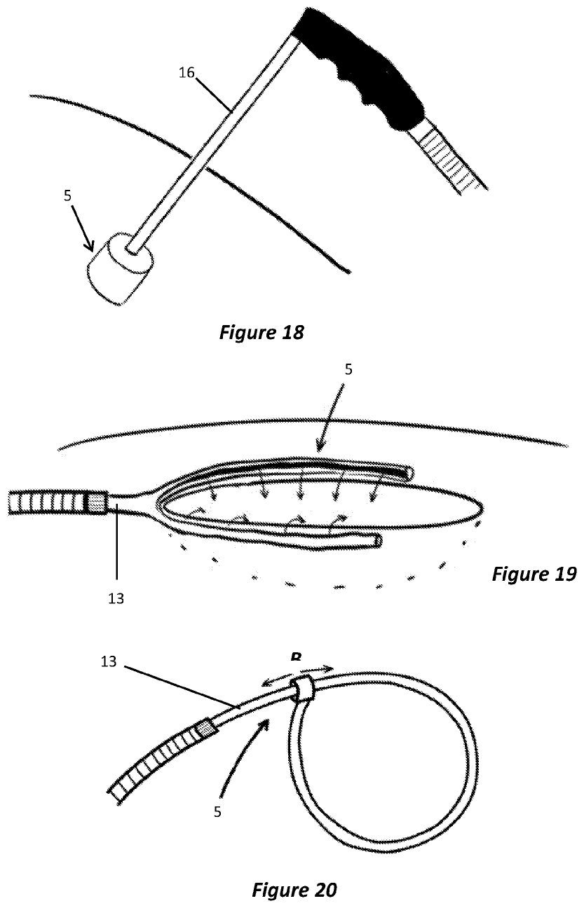

In one embodiment the patient interface 5 is adapted to be connectable to a surgical instrument. The surgical equipment may be an existing instrument, or specifically provided for the patient interface 5. An example is shown in FIG. 18. The interface outlet 14 of the patient interface 5 is located at the distal end of a shaft 16, the shaft 16 being connected to a handle at an opposing end thereof. The handle may comprise a grip that conforms to the user's hand. The patient interface 5 may be formed by a number of different methods, for example, as a porous medium, a multi-outlet body, or an expanded outlet.

In some embodiments the patient interface 5 integrates with an existing surgical instrument, such as, for example, a handheld retractor, electro-cautery tool, clamp, or vessel sealing device. In some embodiments, the patient interface 5 may be removably attached to the side of the surgical instrument.

The shaft 16 may be formed of a rigid material such as a plastic or metal. The shaft 16 may have a degree of flexibility. For example, the shaft 16 may be formed of a flexible material (or include one or more zones of flexible material). The flexible material may be formed of a material that retains to a desired shape.

In one embodiment the patient interface 5 comprises one or more arms that each comprises one or more interface outlets 14 or a plurality of outlets for the patient interface 5. An example of this patient interface 5 is shown in FIG. 19. The patient interface 5 preferably is located at or adjacent the wound edge. The patient interface 5 may surround a portion at least of the wound edge. In one embodiment the patient interface 5 comprises one or more separate arms.

The patient interface 5 may have arms in a bifurcated configuration to resemble a “Y”-shape as shown in FIG. 19. The bifurcated configuration of the arms allows the arms to provide better coverage of the perimeter of the surgical cavity 6. The patient interface 5 may comprise a slidable collar as shown in FIG. 20 to provide a patient interface 5 that forms a loop, and that resembles a “b” or “d” shape. The loop may be of a fixed size, or the sliding collar may provide for an adjustable size as shown in FIG. 20. Advantageously, an adjustable loop enables configuration to suit different wound sizes. The adjustable loop may comprise a sleeve or hoop that provide for hoop adjustability B. For example, the patient interface 5 may be an elongate flexible tube having an interface inlet tube 13 for connection to the delivery assembly 4 and a distal end. The distal end may be attachable to a sleeve or hoop through which the inlet tube is passed to create a loop. The interface inlet tube 13 of the patient interface 5 is connected to the delivery assembly 4. The interface outlet 14 of the patient interface 5 may be located in or on the internal surface of the loop and comprise one or more apertures, slits or bands of porous material.

The arms of the patient interface 5 may be formed from a rigid material. In an alternate embodiment the arms are formed from a flexible material. The flexible material may retain a shape or configuration when subjected to plastic deformation

The interface outlet 14 of the patient interface 5 may be located in one or more zones located in the arms of the patient interface 5 that, when in use, will surround the surgical cavity 6. For example, where the arms of the patient interface 5 have a tubular configuration, the outlet may be located on the face or side of the arm that faces the surgical cavity 6, when in use. This means that the interface outlets are generally located in or on the inner portion of the patient interface 5. The interface outlet 14 of the patient interface 5 may comprise one or more apertures, slits or bands of porous material. For example, as shown in FIGS. 19 and 20, the patient interface 5 when in position surrounds the surgical cavity 6 at least in part, and the portion of the patient interface 5 that is adjacent the surgical cavity 6 comprises at least one outlet such that, in use, gas flow is directed into the surgical cavity 6.

As per previous embodiments, the patient interface 5 may comprise an interface inlet tube 13 for gas flow received from the delivery assembly 4. The patient interface 5 may include an interface inlet tube 13 that extends from one or more of the arms of the patient interface 5 that is adapted for connection to the delivery assembly 4.

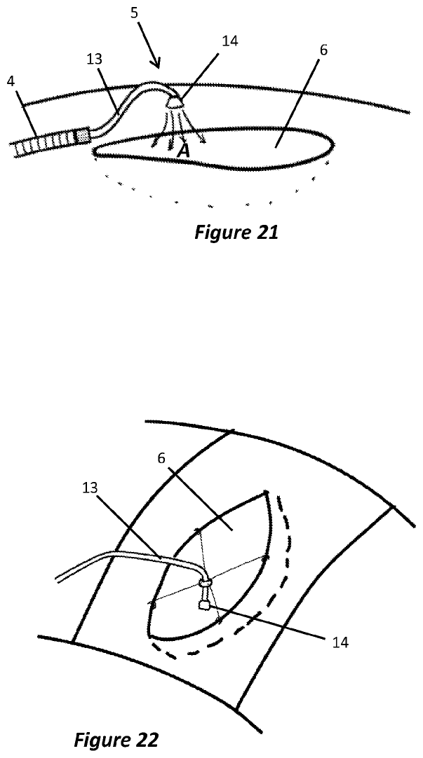

In one embodiment the patient interface 5 comprises at least one elongate interface inlet tube 13, adapted to locate above the surgical cavity 6, and wherein the interface outlet 14 of the patient interface 5 is directed downwards toward the surgical cavity 6. An example of this is shown in FIG. 21 where “A” denotes the gas flow from the interface outlet 14 of the patient interface 5.

The interface inlet tube 13 in the form of a flexible neck may be adapted to locate above the surgical cavity 6 by being bent to form a raised orientation, or may be articulated to form a raised orientation. The interface inlet tube 13 may maintain its shape by being formed of a deformable material that is resilient, such that the material retains its shape when subjected to plastic deformation, or through use of a suitable mechanism such as a series of ball/socket connections, corrugations or pliable material selection.

The interface inlet tube 13 may form part of the patient interface 5 with the interface outlet 14 of the patient interface 5 located at the end of the interface inlet tube 13. The interface inlet tube 13 of the patient interface 5 connects to the delivery assembly 4.

The interface inlet tube 13 allows the user to decide on placement throughout the surgery and maintains the patient interface 5 away from the surgical working space. This patient interface 5 design delivers gases downwardly into the working space of the surgical cavity 6 from a position external to the working space of the surgical cavity 6, including when the patient is positioned at an angle relative to horizontal on the operating table. The gas flow may have a density greater than air to provide effective filling of the surgical cavity 6.

In one embodiment gas flow having a density greater than air is used where the interface outlet 14 is positioned above the surgical cavity 6.

The patient interface 5 may be located above the surgical cavity 6 by being suspended above the surgical cavity 6. For example, FIG. 22 shows a patient interface 5 suspended over the surgical cavity 6, and wherein the patient interface 5 is positionable by at least one cable being attached to opposed sides of the surgical cavity 6. The one or more cables may be attached to an interface inlet tube 13 that connects to the patient interface 5. The interface inlet tube 13 may have attachment means that connect the interface inlet tube 13 to the one or more cables. In one embodiment the connection means may be a ring or sleeve that is located about the interface inlet tube 13. The cable(s) may be connected directly to the ring or sleeve. In one embodiment the suspension system may include two pairs of cables located on opposed sides of the surgical cavity 6. The cables may be formed of any material to suspend the patient interface 5, such as metal, string, rope or suitable polymeric materials.

The cables may be attached to the surgical cavity 6 with a clip at or adjacent the wound edge. The cables may be attached to the wound edge using adhesives, glues, or staples. The cables may be tensioned to fix the height of the patient interface 5 above the surgical cavity 6. The length of each cable may be adjustable so that they can be manipulated to alter the position of the patient interface 5 over the surgical cavity 6.

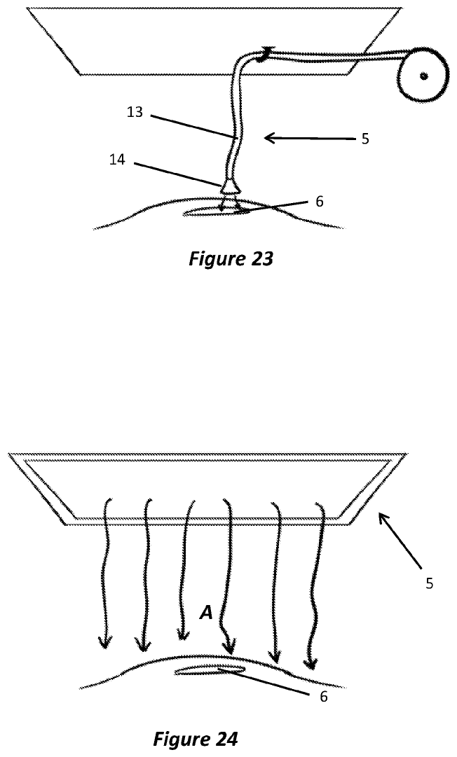

In one embodiment the patient interface 5 is suspended above the surgical cavity 6. For example, FIG. 23 shows a patient interface 5 suspended over a surgical cavity 6 with the interface inlet tube 13 attached to the ceiling to hold the patient interface 5 in position. This embodiment may provide a flexible height adjustment allowing a user to decide on placement throughout the surgery as well as maintaining the patient interface 5 away from the surgical site. The patient interface 5 may connect to a reel that varies the length of the interface inlet tube 13 as required, similar to a retractable hose.

In one embodiment the patient interface 5 may be integrated into, or form part of, a vent that is positioned above the surgical site. For example, shown in FIG. 24 is a vent that may be located in the ceiling above the surgical site. The gas flow system may be integrated or connected to the vent. The vent may receive gas flow from the gas source 2, 3 and then direct the gases downwardly to the surgical site via the vent. In some embodiments the patient interface 5 may be integrated to, or connects into, an existing air conditioning or ventilation system. The medical gases delivery system 1 may be incorporated into a theatre down flow system, as opposed to a bespoke or separate system. The medical gases delivery system 1 may deliver specific gases, as opposed to conditioned air.

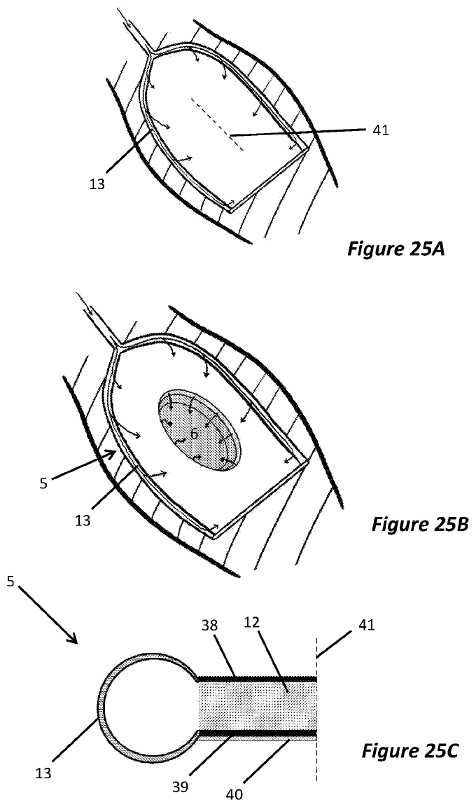

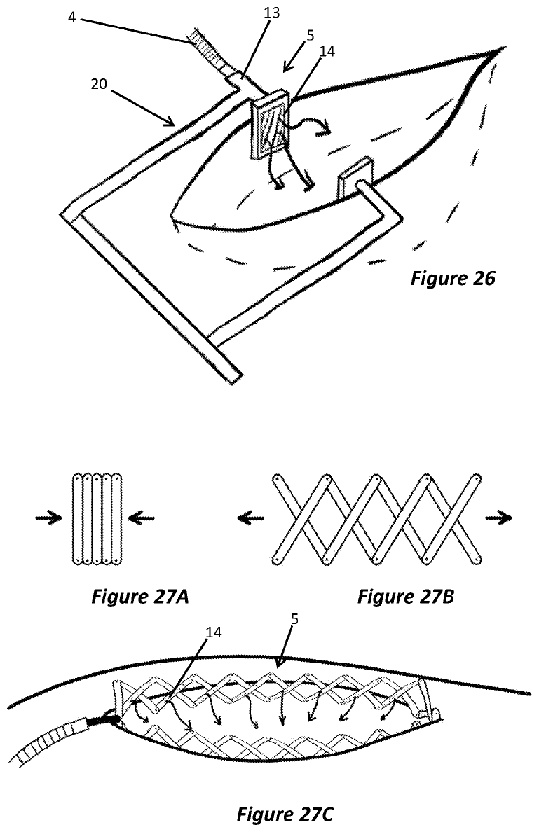

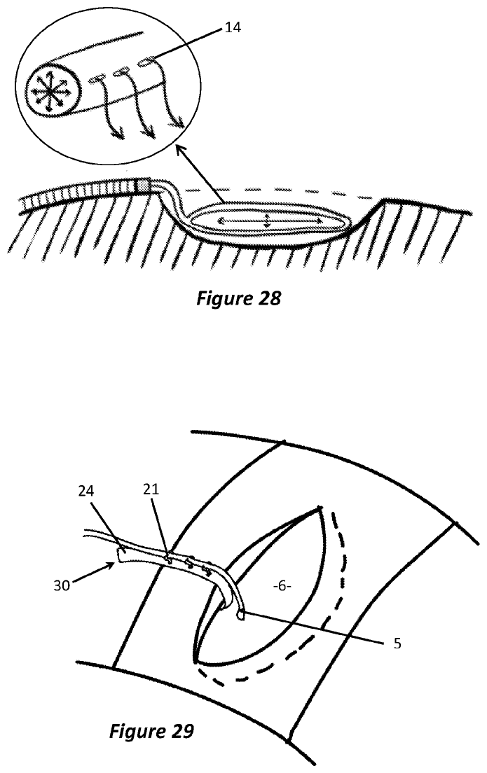

In one embodiment the patient interface 5 forms part of a surgical drape. As shown in FIG. 25A, a surgical drape is placed over the intended site of the surgical cavity 6. The surgical drape may comprise two or more layers. The surgical drape may comprise an upper 38 and lower layer 39 with a layer of gas permeable substrate 12 interposed between the two layers. The gas permeable substrate 12 may be a porous medium such as a fabric layer. The upper 38 and lower 39 layers may be comprised of a substantially gas-impermeable material. The lower layer 38 may include an adhesive layer 40 or adhesive portions to attach the drape to skin of the patient. The surgical drape may include one or more gas pathways. The gas pathways may be incorporated into the surgical drape, such as through the gas permeable substrate 12.

The surgical drape may be marked with an intended line of incision 41. In use, the surgical drape can be placed on the patient such that the intended line of incision 41 is located on top of the intended surgical site. The surgeon can make an incision through the layers of the drape to enter the surgical site and to create the surgical cavity 6. The making of the incision through the gas permeable substrate layer 12 effectively creates outlet(s) 14, located adjacent the wound edge, as shown in FIG. 25b. The interface outlet(s) 14 provides gas flow into the surgical cavity 6. The outlet(s) 14 may be located adjacent the wound edge. The outlet(s) may provide gas flow into the surgical cavity.

The interface inlet tube 13 extends into the surgical drape and provides gas flow to the interface outlet 14 of the patient interface 5. The interface inlet tube 13 may be interposed between the upper and bottom layers and/or into the gas permeable substrate layer. The interface inlet tube 13 extends out of the surgical drape to connect to the delivery assembly 4.

Incorporating the patient interface 5 into the surgical drape provides for a system that is self-positioning and provides little or no inconvenience to the surgical team. The drape may also include its own heating system to warm gas circulating in and/or flowing through the gas permeable substrate layer. The heating system may therefore effectively warm gas throughout the gas permeable substrate layer, providing heating to a large surface area of the drape and hence to the region of the patient located beneath the drape. The heating system may also contribute to conditioning of the gas. Some embodiments may include gas permeable substrate layer throughout a majority or substantially the entirety of the drape. In such embodiments, heating of the gas introduced into the gas permeable substrate layer can effectively provide patient warming once the drape is placed over the patient body.

In some embodiments the surgical drape may be attached to the patient or other suitable equipment, such as surgical retractor. The drape may stick directly onto the patient and may be designed to be cut through when making the initial surgical incision. This advantageously allows the drape to be used with or sized for a range of wound sizes and shapes.

The medical gases delivery system 1 may also include a surgical retractor or surgical retraction system.

In some embodiments the patient interface 5 is connected to the surgical retractor 20 by an attachment mechanism (such as an attachment mechanism as described below) or integrally formed with the surgical retractor 20 (e.g. see FIG. 26).

In some embodiments the surgical retractor 20 includes a heating system which can be incorporated into the surgical retractor or provided upstream of the surgical retractor. The heating system allows the gas flow to be conditioned to a certain temperature, reducing or preventing condensation, and can be configured to heat the patient and/or maintain a desired patient body temperature.

Various forms of retractors 20 are described herein and may be used in combination with any one or more of the diffusors and attachment mechanisms described.

FIG. 26 provides an example of a self-retaining retractor 20 having one or more adjustable arms that can be adjusted and locked to a width suitable to hold the wound edges and/or walls of the surgical cavity 6 apart. As shown the patient interface 5 can be incorporated into the retractor such that the patient interface 5 forms part of the retractor blades. The retractor 20 may include one or more gas flow paths within or attached to the retractor 20. As shown in FIG. 26, the retractor is formed of a tubular material that provides for the gas flow path. The interface inlet tube 13 is then connectable to the delivery assembly 4 such as by a threaded connection or other suitable connection means.

In one embodiment the patient interface 5 is removably connected to the retractor 20 by a suitable connection or fastening means. This may assist in allowing the retractor 20 to be reusable and resterilizable, providing for the patient interface 5 to be replaced as needed. Alternately, the portion of the retractor 20 comprising the gas path may attach to the retractor 20, the portion of the retractor 20 providing the gas path being replaceable. Advantageously, the patient interface 5 can be used on different types of retractors.

Any of the embodiments of the patient interface 5 described above can be utilized with or attached to the retractor. A benefit of this system is that the patient interface 5 can be attached at a range of positions on the retractor 20 to provide for positioning of the patient interface 5 outside of the surgical cavity 6, within the surgical cavity 6, or spanning the surgical cavity 6 as required.

As shown in FIG. 27A to 27C the retractor may be in the form of an expandable framework or linkage system. Such a system is adjustable to fit a wide range of surgical cavity 6 sizes and combines two functionalities in a single device. The retractor may be lockable so that it holds its position within the surgical cavity 6. The expandable framework or linkage system is adapted for radial extension and retraction within the surgical cavity 6. The body of the expandable framework or linkage system may be formed from a tubular material to provide for a gas flow path within the expandable framework or linkage system. The expandable framework or linkage system may include one or more interface outlets 14 for the patient interface 5. For example, the interface outlets 14 can be distributed the length of the expandable framework or linkage system so that gas flow is introduced into the surgical cavity 6 from a range of discrete entry points. The expandable framework or linkage system may have an interface inlet tube 13 that is connectable to the delivery assembly 4.

The expandable framework or linkage system may position in the surgical cavity 6, such as at the wound edge, or wholly within the surgical cavity 6 to retain the walls of the surgical cavity.

As shown in FIG. 28 the retractor may be in the form of an inflatable conduit. The inflatable conduit may be an inflatable tube or provided in any other suitable configuration. The conduit may have a circular or elliptical cross section. The inflatable tube may be positioned inside the surgical cavity, whether at the margin or wholly within the surgical cavity to retain the walls of the surgical cavity 6. The tube can be inflated to apply an outward and/or opening force to the walls of the surgical cavity 6. The tube may comprise one or more outlets 14 for the patient interface 5 in the surface of the inflatable tube, the inflatable tube having an inlet that is connectable to the delivery assembly. Alternately the patient interface 5 may be attached to the surface of the inflatable tube, such as with an attachment mechanism as described below.

In one embodiment the inflatable retractor is formed as an elongate tube having a connection mechanism at the distal end of the tube so that it can be formed into a donut shape.

The body of the inflatable retractor may have one or more interface outlets 14 for the patient interface 5 spaced along the surface of the inflatable retractor. The interface outlets 14 may be in the form of holes or slits as shown in FIG. 28.

### 4. Attachment Mechanism

Without some method of securing the medical gases delivery system 1 to the environment around the surgical cavity 6, any one or more of the components of the medical gases delivery system 1 (e.g. the patient interface 5 or delivery assembly 4) may become prone to movement. This can cause frustrations to the surgeon as components impinge on their working space. Movement of components of the system can also cause gas flow to be directed into tissues or body fluids that cause unwanted noise or splatter and move into gas pockets which can inflate and cause organ movement (see paragraph [0297] above). Effective retention of the patient interface 5 or delivery assembly 4 greatly reduces the likelihood of these problems occurring and improves the usability of the system.

The patient interfaces 5 described can be positioned relative to the surgical cavity 6 by an attachment mechanism 30. The attachment mechanism 30 attaches to any one or more of the delivery assembly 5 or interface inlet tube 13, and to a position on the surgical site. The attachment mechanism can attach to the patient interface 5 (for example, to the interface inlet tube 13 or the body of the patient interface 5) or the delivery assembly 4 to hold the patient interface 5 in a desired position. The attachment mechanism 30 may attach the patient interface 5 or the delivery assembly 4 (or both) to the patient, retractor, surgical drape, surgical instrument or surgical equipment. For example, the described attachment mechanisms 30 may be separate to or unitary with a retractor or surgical equipment.

Various attachment mechanisms 30 for the patient interface 5 systems described above are now described. Any one of the attachment mechanisms 30 described below can be used with any of the above described patient interface 5 embodiments.

In one embodiment the attachment mechanism 30 may be provided as a malleable structure, such as a malleable hook. The attachment mechanism 30 may advantageously be part of or comprise a retractor or retractor system. One example is shown in FIG. 29 and comprises an elongate body 24 with one or more clip structures 21 for the delivery assembly 4 and/or patient interface 5. The elongate body 24 may be formed of a malleable material and may take the form of a malleable retractor, such as those known in the art. The elongate body 24 may be shaped to hook around the wall of the surgical cavity 6, with a portion extending externally to the surgical cavity 6 to span the wall of the surgical cavity 6. Thus, the elongate body 24 may be in the form of a paddle or blade that can be bent or otherwise configured to be positioned on top of the edge of the surgical cavity 6, tucking under the wound edge. The attachment mechanism 30 may be attachable to the delivery assembly 4 and/or patient interface 5 and can include any mechanism described herein including clips or brackets to hold the delivery assembly 4 and/or patient interface 5 in place. In the embodiment shown in FIG. 29, the attachment mechanism 30 comprises a series of clip structures 21 that project outwardly from a surface of the elongate body 24. The clip structures 21 may be configured to receive a portion of the patient interface 5, such as the interface inlet tube 13 and hold the patient interface 5 in a desired position adjacent and/or in the surgical cavity 6. The clip structures 21 may be configured to receive a portion of the delivery assembly 4, to hold the patient interface 5 in a desired position adjacent and/or in the surgical cavity 6.

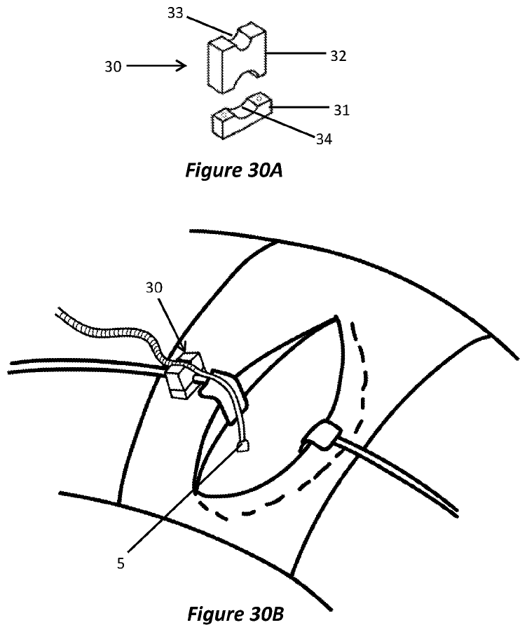

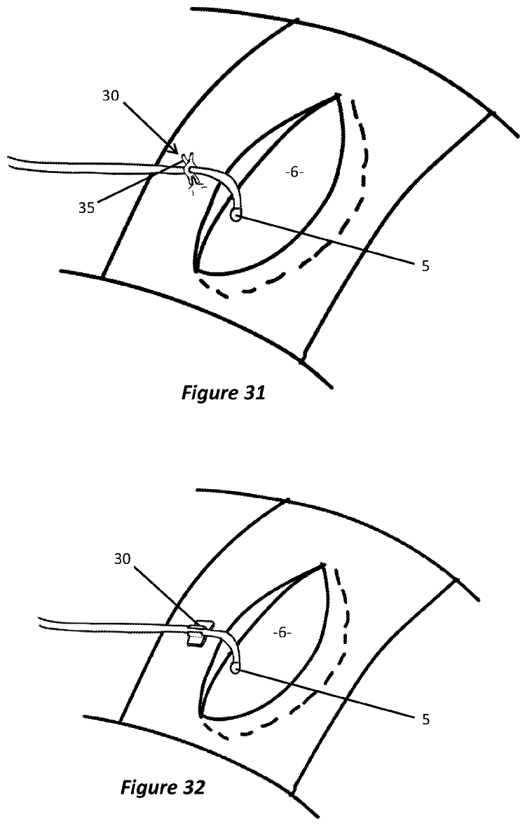

As shown in FIGS. 30A and 30B the patient interface 5 may incorporate a two-part clip or bracket. The clip or bracket may support the connection of the delivery assembly 4 and/or the patient interface 5. The connection for the delivery assembly 4 and/or the patient interface 5 may be provided for by an attachment mechanism 30 in the form of a clip having a base 31, and a bracket 32 interposed over the base 31, wherein the clip comprises an aperture 34 between the clip and bracket 32. The aperture 34 is configured to receive and retain a retractor 20 such as by engaging an arm of the retractor 20. The clip may additionally comprise a slot 33 to receive a portion of the delivery assembly 4 or interface 5, such as tube 13, assisting in retaining the interface 5 in a desired position adjacent and/or in the surgical cavity 6.

In one embodiment the bracket 32 of the clip connects to the base 31 by magnetic interactors, although other methods could be used such as the use of adhesive or friction fit.

The clip may include a rotating joint, wherein the clip is attached, (e.g. with adhesive), to the surgical drape, patient, retractor, or surgical instrument, the rotating joint allowing the two-part clip to adjust its position to facilitate positioning and/or movement of the patient interface 5 in the surgical cavity.

In one embodiment the patient interface 5 or delivery assembly 4 (or both) are held by an attachment mechanism 30 in the form of a ring clip element. As shown in FIG. 31 the ring clip element is fastened or clipped to a surface such as the surgical gown/drapes or the patient's skin. The ring clip element may be formed with an aperture through it, for example, to locate the patient interface 5 or the delivery assembly 4 (or both). In the embodiment shown in FIG. 31, the ring clip element comprises a ring with a central aperture, the ring configured to encircle a portion of the interface 5. The ring clip element may further include a pair of fastening arms 35 extending outwardly from the ring. The aperture may be located at the pivot point of the fastening arms 35. In one embodiment the ring clip element may be spring loaded. The ring of the ring clip element may be formed of a flexible material that the patient interface 5 or delivery assembly 4 (or both) can be pushed through to help retain position, while still maintaining some degree of freedom for positioning adjustments relative to the clipped position. Alternatively, the ring and fastening arms may be integrally formed with the interface 5.

The ring clip element may have a clamping surface formed of a soft material, such as foam, rubber or silicone. The use of soft materials may provide for better grip, or improved safety as the soft material may reduce the likelihood of applying excessive force,

The fastening arms 35 of the ring clip element may be biased to a clamped or closed position. For example, the ring clip element may be spring loaded or be mechanically biased to the clamped position.

The attachment mechanism 30 may alternatively comprise a suction cup to attach to the surgical site, for example to the patient, or to the surgical drape. The suction cup may comprise a holder or ring to attach the patient interface 5, delivery assembly 4 or both.

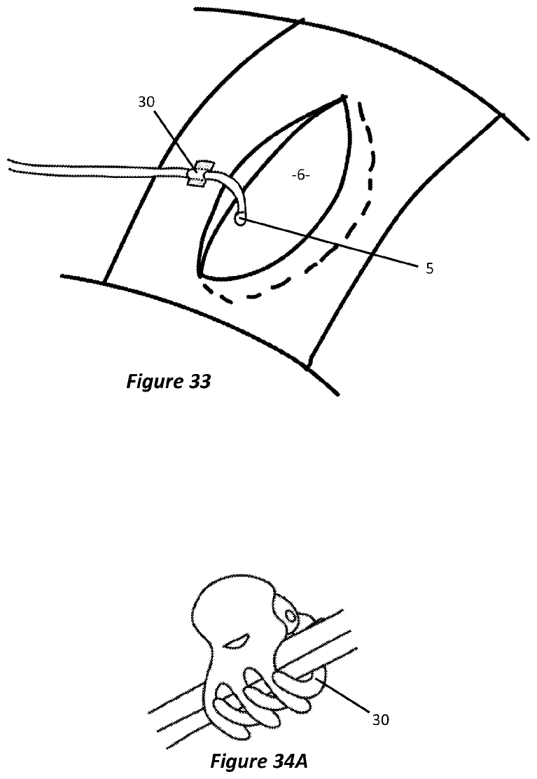

Referring to FIGS. 32 and 33, there are shown embodiments of attachment mechanisms for the patient interface 5 or the delivery assembly 4 (or both) comprising a clip for attachment to a patient's skin, retractor, surgical gown, surgical instrument or furniture. The clip includes a base portion and an interface engaging portion. The base portion may have an adhesive surface for attachment to the desired surface, e.g. skin. The adhesive may be chosen based on the surface that the clip is to be attached to. The base portion may include a suction cup, glue, tape pads or Velcro to hold the clip in place. The clip may alternatively or additionally be attached to the patient's skin, surgical cavity, or surgical drape with sutures, surgical staples or pins. The interface engaging portion of the clip may engage and retain the conduit or tube into place. The interface engaging portion may partially or fully surround the interface inlet tube 13 or a patient interface conduit 11 of the delivery assembly 4.

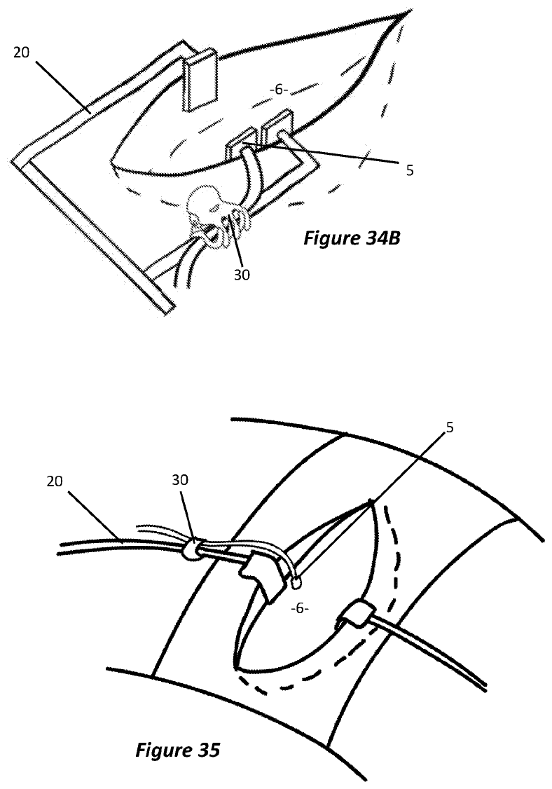

In one embodiment the attachment mechanism 30 for the patient interface 5 or the delivery assembly 4 (or both) may be a clip fastener as shown in FIG. 34A or 34B having one or more interlocking features at its distal end to engage with the patient interface 5 or the delivery assembly 4 (or both) to a retractor, surgical drape, surgical instrument or surgical furniture.

The clamping surface of the clip may use foam, rubber or silicone. The clip may be biased to a closed position, for example with a spring or mechanical closure.

The clip may be a separate component to the patient interface 5 and/or delivery assembly 4. The clip may first be attached to a first component such as a retractor, delivery assembly 4, or interface inlet tube 13. The clip is then attached to another of the components.

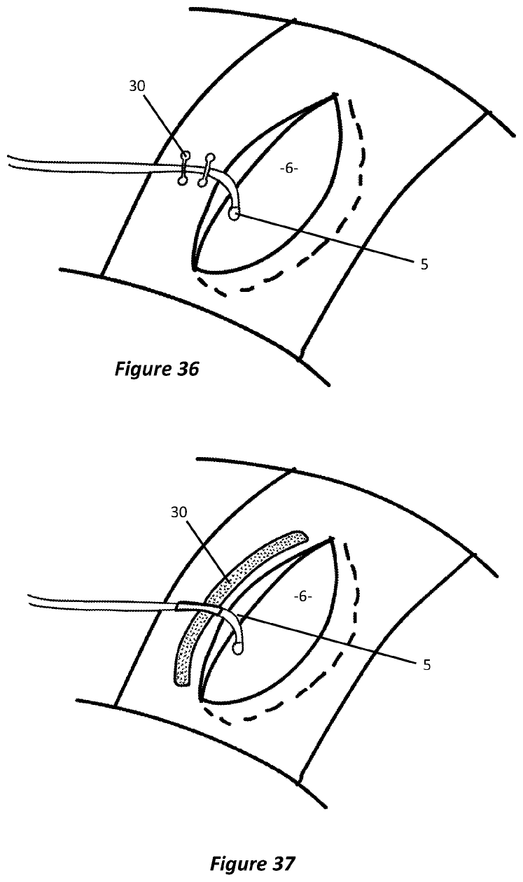

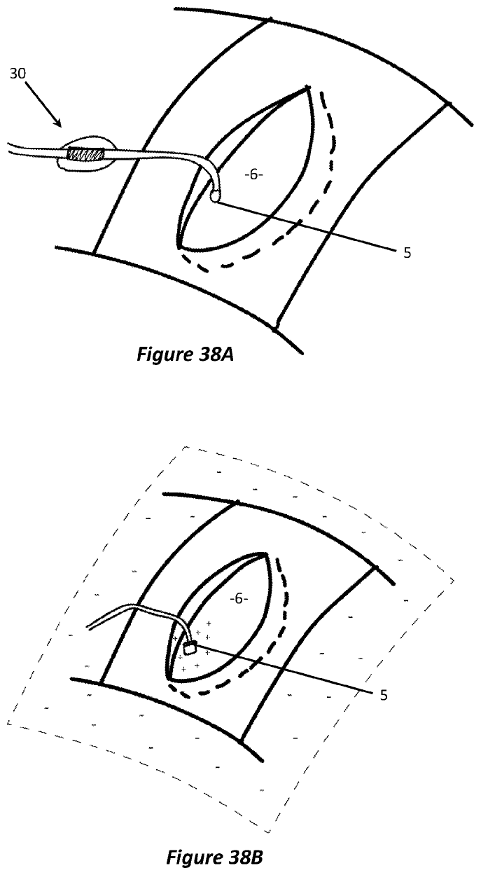

In one embodiment the attachment mechanism 30 is a clip, a tie, a band, a releasable mechanical fastener such as a hook and loop structure (e.g. Velcro® strap), an elastic strap, bent wire or knotted string. As shown in FIG. 35 the attachment mechanism 30 may attach the patient interface 5 or the delivery assembly 4 (or both) to a retractor, a surgical instrument or surgical furniture.

In one embodiment the patient interface 5 or the delivery assembly 4 (or both) could include a unitary bracket that allows a strap, tie, wire or string to pass through in a desired orientation. For example, the delivery assembly and/or patient interface 5 may include a projection with a slot or aperture provided therein to allow insertion of the strap, tie, wire or string. The aperture or slot may be formed by an arm that loops from a surface of the delivery assembly or patient interface 5 to form the aperture or slot. The arm may attach to the delivery assembly or patient interface 5 at one end only to allow the strap, tie, wire or string to be slipped into the aperture or slot.

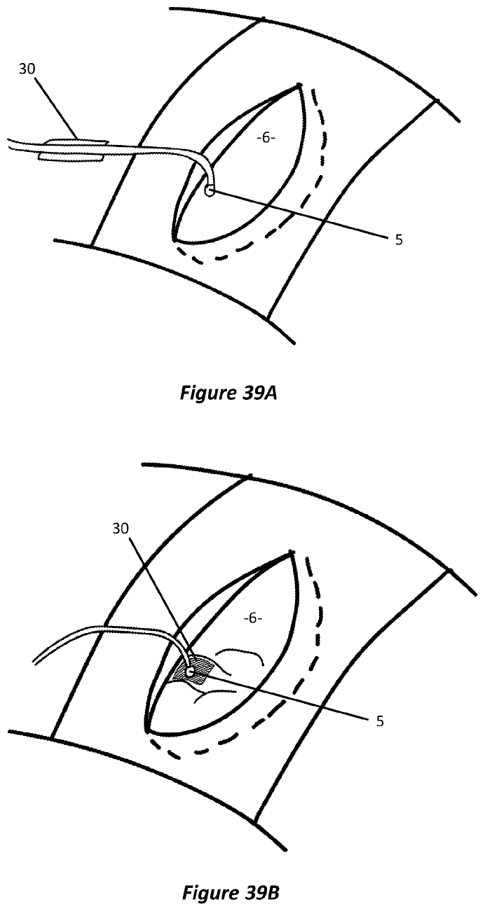

In one embodiment the attachment mechanism 30 is a resilient adhesive tie that retains the patient interface 5 or the delivery assembly 4 (or both) to a retractor 20, a surgical instrument or surgical furniture as shown in FIG. 36. The ties may be pre-assembled with the patient interface 5 and/or the delivery assembly 4. The sticky ties may be surgical tape that holds the patient interface 5 or the delivery assembly 4 (or both) to a retractor 20, a surgical instrument or surgical furniture. A water or alcohol soluble glue may be washed away after surgery.

As shown in FIG. 37 the retention mechanism 30 may comprise an attachment strip comprising complementary releasable mating structures (such as a releasable mechanical fastener e.g. hook and loop structure, or ball lock structure) having an adhesive surface for attachment to a surgical drape, the patient, a surgical tool or furniture, and a corresponding attachment strip about the delivery assembly 4, the patient interface 5 or the delivery assembly 4 and patient interface 5.

The attachment strip may extend adjacent the surgical cavity 5. The attachment strip may comprise a single strip or a plurality of smaller strips. The attachment strip may allow for detachment of the complementary mating structures allowing re-positioning of the attachment strip.

As shown in FIGS. 38A and 38B, in one embodiment the retention mechanism 30 comprises a magnetic strip or pad for positioning on, or about, a surgical drape, the patient, a surgical tool or furniture, and a corresponding mating element (i.e. magnetic or ferromagnetic element) about the delivery assembly 4, the patient interface 5 (or both). The magnetic pad may comprise an adhesive surface that sticks to the surgical drape, the patient, a surgical tool or furniture. The magnetic element about the delivery assembly 4 and/or the patient interface 5 connects to the magnetic pad.

The magnetic pad may be placed below the patient's body. A patient interface 5 comprising a ferrous material or other magnetic metal when placed in the surgical cavity 6 may then be located into position by the magnetic forces from the magnetic pad.

The delivery assembly 4 and/or patient interface 5 may comprise the magnetic material, with the mating element positioning on, or about, a surgical drape, the patient, a surgical tool or furniture.

As shown in FIGS. 39A and 39B the retention mechanism 30 may be an adhesive pad or strip that comprises adhesive on both the top and lower surfaces of the adhesive pad or strip. The adhesive pad or strip may be attached to a surface adjacent the wound edge as shown in FIG. 39A, or the surgical site outside of the surgical cavity 6 as shown in FIG. 39B. The sticky pad can be formed over the delivery assembly 4, the patient interface 5 (or both) and secure to a surgical drape, retractor, the patient, a surgical tool or furniture.

As shown the retention mechanism 30 may be an adhesive pad or strip that sits within the surgical cavity 6. The patient interface 6 may then attach to the adhesive pad or strip.

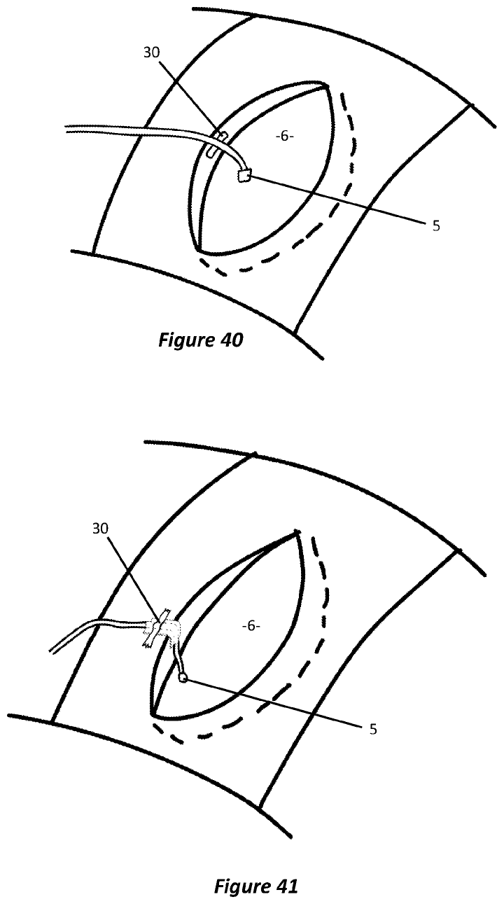

As shown in FIG. 40 the patient interface 5 or the delivery assembly 4 (or both) may include a retention mechanism 30 that uses glue or other suitable surgical or medical adhesive. For example, the retention mechanism 30 may be a pad or band formed unitarily with the patient interface 5 or the delivery assembly 4 (or both) and glued to a surface of the patient, surgical cavity, a surgical drape, a surgical tool or furniture. A surface of the patient interface 5 or delivery assembly 4 may have a substantially planar surface to support attachment. The glue may be a water-based glue that can be removed after the surgery. For example, the glue may be dissolvable to assist in removal after surgery.

As shown in FIG. 41 the retention mechanism 30 may comprise a cover that extends over the delivery assembly 4, the patient interface 5 (or both), the cover being attachable to a surgical drape, or patient surface by a suitable fastener (e.g. glue, sutures or adhesive). The cover may be formed of a rigid or resilient (such as silicone) material. Alternatively, the cover may be formed of a soft and pliable material. The cover may be moulded to form about or over the wound edge. The cover may form a protective shell which may protect the patient interface 5 or delivery assembly 4 from damage by surgical tools in the surgical cavity 6.

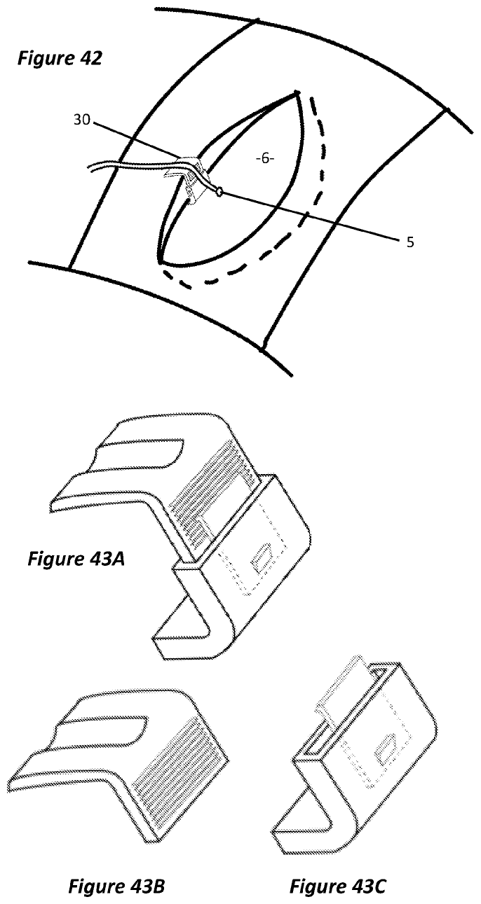

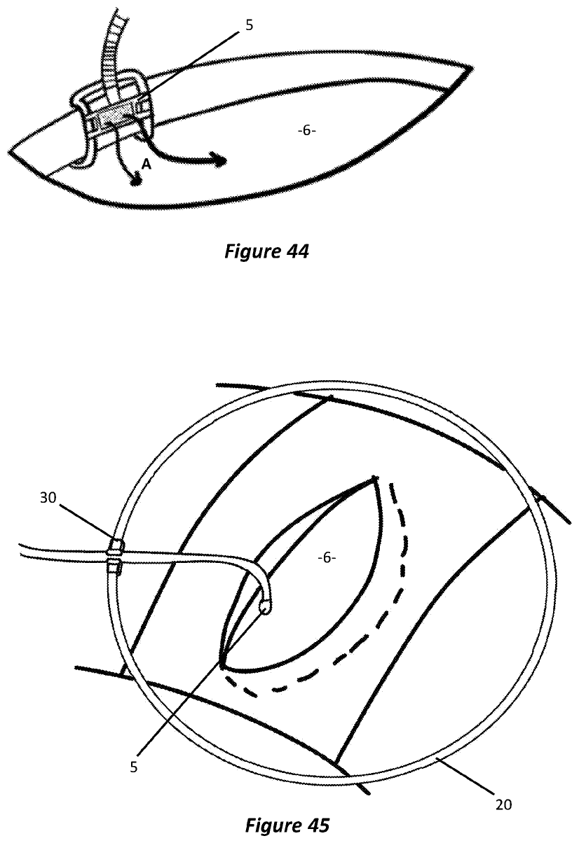

As shown in FIGS. 42 and 43A to 43C the retention mechanism 30 is a clamp for attachment about the patient wall of the surgical cavity, the clamp comprising an attachment for the delivery assembly 4, the interface or both. The clamp may be height adjustable, for example by use of a ratchet mechanism as shown in FIGS. 43A to 43C.

The clamp may comprise two complementary pieces that combine, with one piece having an aperture or slot, and the other piece a complementary projection. As shown in FIGS. 43A to 43C the clamp may install at the wound edge extending into the surgical cavity 6. A benefit of the two-part clip is that it can be applied to a variety of wound wall thicknesses. The clamp may include an attachment device for the delivery assembly 4, the patient interface 5 or both the delivery assembly 4 and the patient interface 5. The attachment device may be selected from any mechanism that can be used to attach the delivery assembly 4 or patient interface 5 to the clamp, and non-limiting examples include a clip, glue, sticky pad, magnets, suction, clips or press fit groove. Alternatively, the attachment device (may be integrally formed with the clamp (not shown). In one embodiment the retention mechanism (i.e. the clamp) may be formed integrally with the patient interface 5.

In one embodiment the clamp comprises one or more magnetic parts. That is, the elements of the clamp may stick together by a magnetic force. For example, a first part having one polarity and a second part having a different polarity, or a first part being a magnet and a second part formed of a ferrous material. The two parts may be placed either side of a structure to be clamped, the magnetic interaction drawing the two parts together to clamp over the desired structure. The clamp may be a spring-loaded clamp. That is, the spring biases the clamp to a closed position such that opening the clamp against the bias, to span the wound wall thickness, fixes the clamp in place. The spring-loaded clamp may rotate about an axis or move two or more rails.

The clamp may include a release mechanism such as a button to displace and release the projection within a slot.

As shown in FIG. 44 the retention mechanism 30 comprises an elongate rail attachable to, or adjacent, the surgical cavity 6, and a clip for attachment to the rail. The clip is moveable along said rail and provides for attachment of the delivery assembly 4, the patient interface 5 or the delivery assembly 4 and patient interface 5. The retention mechanism 30 may be integrated into a retractor 20. The delivery assembly 4, the patient interface 5 or both the delivery assembly 4 and the patient interface 5 preferably attach to the tracks of the rail, and preferably in a slidable arrangement. The rail may comprise two vertically spaced tracks for attachment of the delivery assembly 4 and/or patient interface 5. The rail may be provided in alternative geometries, such as in the form of an “L” shape or the vertically spaced tracks may be curved and arranged to approximate a ring structure. The tube or conduit of the delivery assembly 4 or patient interface 5 may be adapted to attach to the outlet of the patient interface 5 that may interpose between the two vertically spaced tracks.

The rail may integrate with a mechanical retraction system. For example, the retraction system may be adapted for attachment of the rail, and wherein the rail can span across a portion at least of the wall of the surgical space.

As shown in FIG. 45, the rail may alternatively be fastened on a surface of the patient surrounding the surgical cavity 6, such that most or all of the rail does not enter the surgical cavity 6. As shown, the circular railing system may be provided in a ring configuration located in the surgical site (outside the surgical cavity 6) such that it surrounds the surgical cavity 6. The circular railing system may rest on the patient or the surgical drape, or a combination thereof.

The circular railing system enables the patient interface 5 to track around the ‘circumference’ of the surgical cavity 6 and be repositioned during surgery as required. In one embodiment the circular railing system may be integrated with a retractor 20.

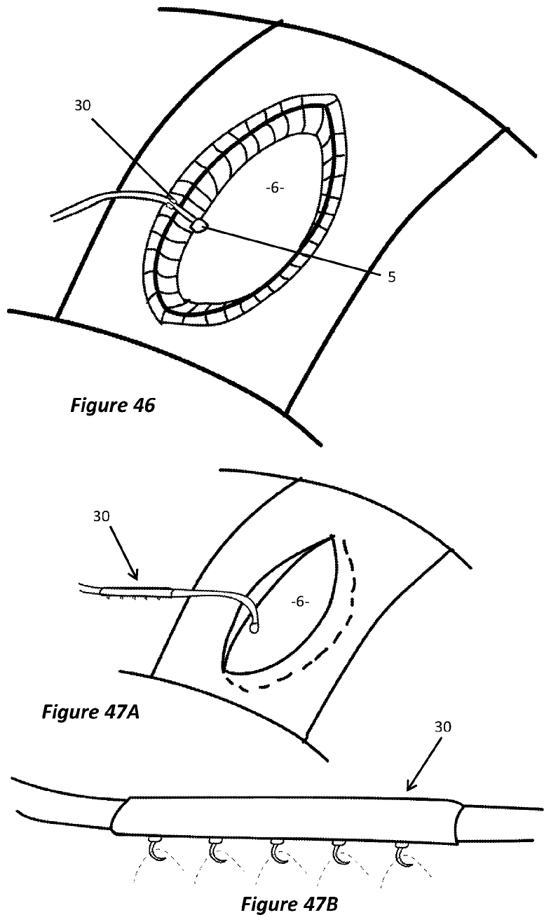

In one embodiment the retention mechanism 30 is attached to a surgical drape, wherein the drape covers at least the wound edge of the surgical cavity 6, and optionally at least a portion of the patient wall of the surgical cavity 6. For example, shown in FIG. 46 is a surgical drape which surrounds the wound edge and may extend into the surgical cavity 6. The surgical drape may include an attachment mechanism 30 located adjacent the wound edge or at the upper patient wall of the surgical cavity 6 for attachment of the delivery assembly 4 and/or patient interface 5. The attachment holds the delivery assembly 4 and/or patient interface 5 in place relative to the surgical drape. Non-limiting examples of possible attachment mechanisms 30 that attach the delivery assembly 4 and/or patient interface 5 to the surgical drape include releasable mechanical fasteners (e.g. Velcro), magnetic devices, suction cups, ties or a clip.

The drape may include a rail system (as discussed above) that may comprise horizontal or vertically spaced rails. For example, if horizontally spaced rails, the rail system may travel along the wound edge of the surgical site, either outside of the surgical cavity 6, or along the patient wall of the surgical cavity 6. The rails may be sewn into the surgical drape or attached for example with adhesive.

In one embodiment the retention mechanism 30 comprises a frame that extends the perimeter of the edge of the surgical cavity 6. The frame may be rigid and attach to a retention mechanism 30 or sit over the surgical cavity 6. In an alternative embodiment, the retention mechanism 30 may comprise a frame that extends at least a portion of the perimeter of the surgical cavity 6. The rigid frame may be incorporated into a surgical drape or may be separate to the drape.

The retention mechanism 30 may be a liner for the surgical cavity 6. For example, the liner may be present as a flexible lattice (such as a net or cross-hatched rope) that extends about the edge of the surgical cavity 6, such as the patient wall of the surgical cavity 6, or the upper patient wall of the surgical cavity 6. In some embodiments the liner may traverse inside and outside of the surgical cavity 6 with an attachment for the delivery assembly 4 and/or patient interface 5.

In one embodiment the retention mechanism 30 comprises one or more projections for passage through a surgical drape. For example, the projections may pass through holes already present in the surgical drape, or pierce through the drape when in use allowing the user to place the retention mechanism where they desire.

As shown in FIGS. 47A and 47B the projections may be in the form of hooks attached to a sleeve formed around the delivery assembly 4 and/or patient interface 5. The hooks can pierce the surgical drape to hold the sleeve in place relative to the surgical drape. In another embodiment the projections may include an attachment means for attachment of a complementary fastening portion once the projections have passed through the surgical drape. For example, the projections may include a threaded portion for attaching to a complementary threaded nut once the projections have passed through the surgical drape.

The retention mechanism 30 may comprise plurality of projections, such as at least 2 to 10 projections. When in use the hooks project into the surgical drape at any orientation, the sleeve then holding the delivery assembly 4 and/or patient interface 5 in place relative to the surgical drape.

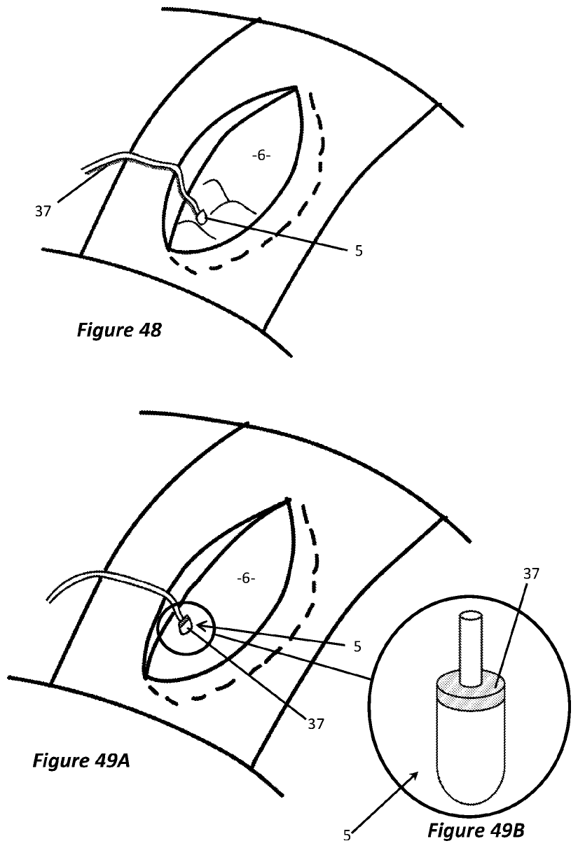

In one embodiment the attachment mechanism 30 comprises a plurality of projections. As shown in FIG. 48 the projections may be less than 5 mm in length, the projections formed from a soft and/or flexible material. The projections may span the delivery assembly 4 and/or patient interface 5, for example by being formed as part of the delivery assembly 4 and/or patient interface 5 or by a sleeve that fits over the delivery assembly 4 and/or patient interface 5. The projections increase the surface area of the interface and consequently creates in increase in friction with other objects. This frictional force is utilised to hold the interface in position around the wound edge or inside the surgical wound. The projections may be formed from a soft material such as soft plastic or silicone.

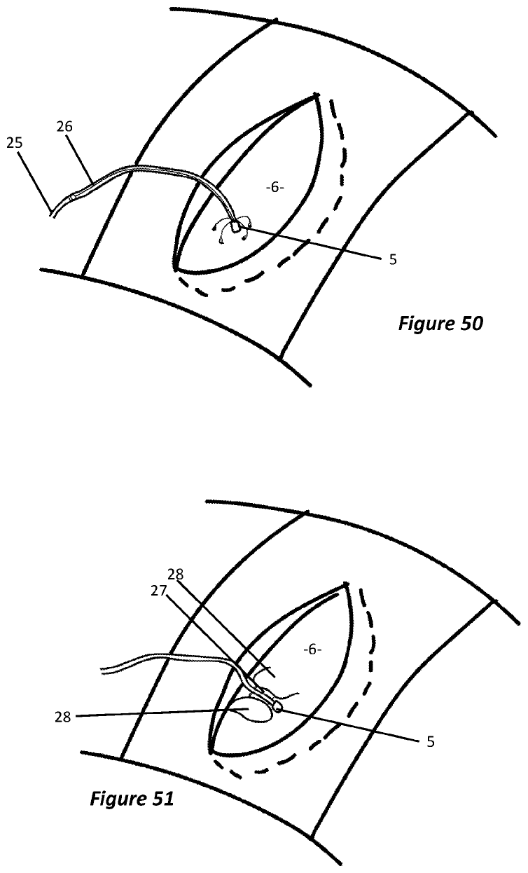

As shown in FIGS. 49A and 49B, the attachment mechanism 30 comprises one or more weighted anchors 37 located on the patient interface 5. The weighted anchor 37 may also be located on the delivery assembly 4 adjacent the patient interface 5 (not shown). The weighted anchor 37 acts to position the patient interface 5 within the surgical cavity when in use. The weighted anchor 37 can be formed of metal, dense plastic or a ceramic. The weighted anchor 37 can form part of the patient interface 5 or be attachable to the interface, for example, using any one or more of the attachment mechanisms as described above.

In one embodiment the attachment mechanism 30 comprises a mechanical claw having two or more fingers, the mechanical claw adapted to include a release mechanism.

As shown in FIG. 50 the mechanical claw may attached to a surface inside the surgical cavity 6, such as the wall of the surgical cavity or an organ. The claw may form part of a sleeve 25 that can be drawn over the delivery assembly 4 and/or patient interface 5. The claw may include a release mechanism, such as a wire 26 that runs to a button for the surgeon to press for gripping/releasing mechanical claw.

In one embodiment the attachment mechanism 30 comprises an inflatable sleeve 27, the inflatable sleeve 27 adapted for positioning inside the surgical cavity 6. The inflatable sleeve may be located between organs 28, or between an organ 28 and the wall of the surgical cavity 6. As shown in FIG. 51 the inflatable sleeve 27 can wrap around the delivery assembly 4 and/or patient interface 5, or the inflatable sleeve may comprise one or more outlets 14 of the patient interface 5 on or in its surface. The inflatable sleeve 27 may be wedged and inflated between the internal anatomy of the patient to fix the position of the patient interface 5 in the surgical cavity 6.

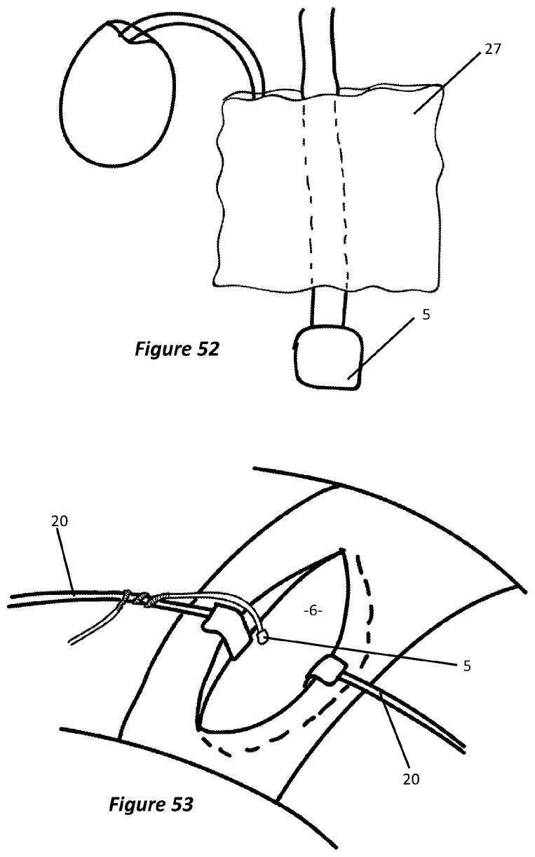

As shown in FIG. 52 the inflatable sleeve may be connected to a pump, e.g. a hand pump, for inflating the sleeve.

In one embodiment the attachment mechanism 30 comprises a flexible region, wherein the flexible region wraps about a retractor, surgical instrument or furniture. As shown in FIG. 53 the flexible region of the delivery assembly 4 and/or patient interface 5 can extend a length of a retractor. The flexible region of the delivery assembly 4 and/or patient interface 5 may be formed from corrugations or a sleeve that has at least a degree of ability to retain a shape or configuration after deformation.

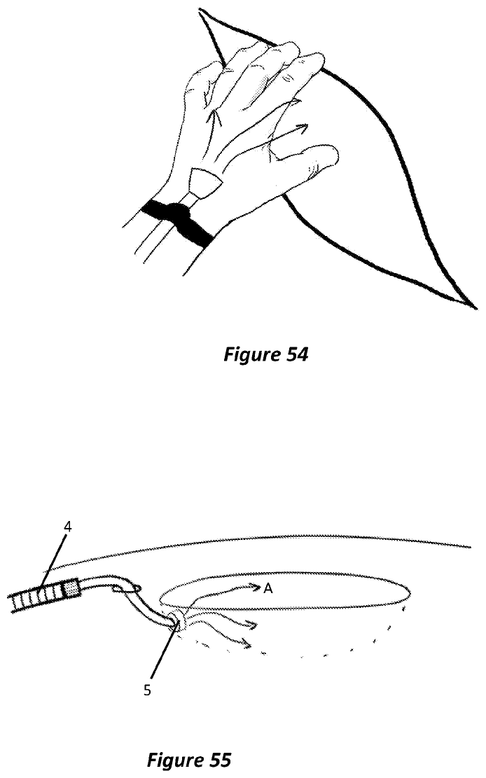

As shown in FIG. 54, the attachment mechanism 30 may allow for attachment of the delivery assembly 4 and/or patient interface 5 to a limb of the surgeon or a surgical tool, such as with a band, strap or clip. The patient interface 5 is positioned on the hand of the surgeon allowing the gases to be delivered into the surgical cavity 6 when the surgeon is working.

As shown in FIG. 55 the patient interface 5 may deliver gas into the surgical cavity 6 via insertion of the patient interface 5 through an incision that creates a passage from the surgical site adjacent the surgical cavity 6 to a patient wall of the surgical cavity 6.

As shown, the gas flow (depicted by “A”) disperses into the surgical cavity 6 from the patient interface 5 located at the patient wall, or adjacent the patient wall.

The patient interface 5 may include an attachment mechanism 30 in the form of a plug, and wherein the plug is adapted for placement inside the incision located in the surgical cavity. An incision made in the side wall of the surgical cavity enables the plug to be inserted and the tube of the patient interface 5 threaded through the plug so that the interface outlet 14 of the patient interface 5 is positioned within the surgical cavity 6.

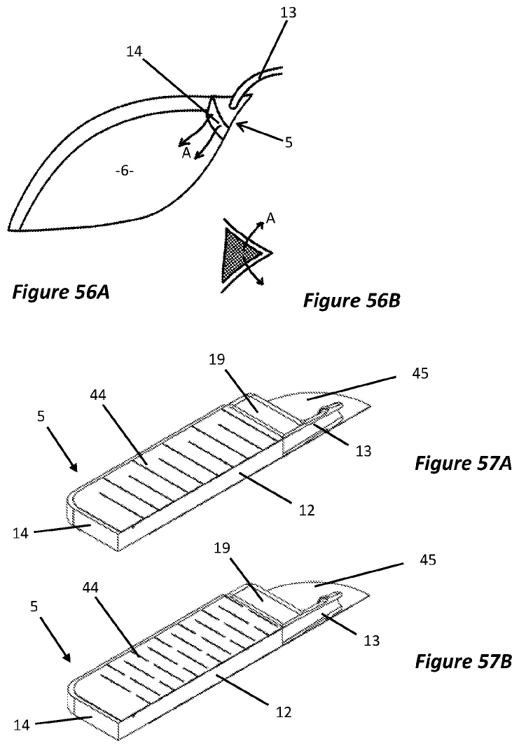

As shown in FIGS. 56A and 56B the patient interface 5 may include a compressible component at or adjacent the patient interface outlet 14 that presses into the surgical cavity. The compressible nature of the compressible component holds the patient interface 5 in place. That is, the component is compressed and then inserted into a particular area. The component then returns to an at least partially uncompressed condition the act of which retains the component in the area. The compressible component may include one or more outlets 14 of the patient interface 5.

As described above, the attachment mechanism 30 may at least in part comprise an adhesive layer, component or region, such as adhesive layer 42 on the rear surface or patient facing surface of the patient interface 5. Such an adhesive layer or component is useful for attaching the patient interface 5 to a variety of surfaces including surface adjacent the surgical cavity 6 or a surface of a surgical retractor. It will be appreciated that there are many types of surgical retractors available for use in a surgical procedure. Some surgical retractors may have minimal or limited surface area onto which an adhesive surface can be reliably attached to. For example, Balfour retractors may have blades formed from a rod of material, wherein the rod is the only surface of the blade onto which anything could be attached. This may not provide sufficient surface area for satisfactory attachment by adhesive.

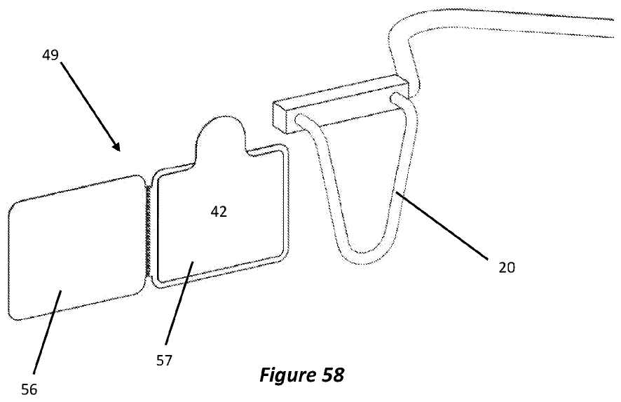

To address this potential problem, the attachment mechanism 30 may comprise a fixation structure 49. The fixation structure 49 is adapted to cooperate with the patient interface 5 to secure the patient interface 5 in place as required for a surgical procedure. An example embodiment of a fixation structure 49 is shown in FIG. 58. The fixation structure comprises a body having first 56 and second portions 57. The first portion is configured to provide a surface onto which an adhesive surface, region or component of the patient interface 5 may attach. In the embodiment shown, the first portion is configured to be positioned on one side, i.e surgical cavity-facing side, of the surgical retractor blade 20. The second portion is configured to be positioned on an opposite, i.e. patient wall side of the retractor blade 20.

The first and second portions may be pivotably attached to each other at respective first ends thereof. The first and second portions can in use, be pivoted or folded such that the first and second portions are placed over each side of the retractor blade 20. The first and/or second portion may have an extension or tab extending outwardly from an edge. In the embodiment shown in FIG. 58, the extension or tab extends from an upper edge of the first portion. The extension or tab may have adhesive to allow folding over and securement of the first and second portions to each other once in position around the retractor blade. Alternatively, or in addition, corresponding surfaces of the first and second portions may be provided with adhesive or other fixation element, e.g. mechanical fastener such as hook and loop. The corresponding surfaces can be attached to each other to retain the fixation structure 49 on the retractor blade 20 and/or to retain the patient interface 5 therebetween.

The first and second portions can be attached at respective first and second ends, forming a sleeve or pocket which can be placed over the retractor blade 20. The fixation structure 49 can be held in place on the retractor blade 20 by adhesive or other fixation element on surfaces of the first and/or second portions as described above. The surgical cavity facing side of the first portion may provide a surface area onto which the patient interface 5 can be attached.

Reference to any prior art in this specification is not, and should not be taken as, an acknowledgement or any form of suggestion that the prior art forms part of the common general knowledge in the field of endeavour in any country in the world.

Where reference is used herein to directional terms such as ‘up’, ‘down’, ‘forward’, ‘rearward’, ‘horizontal’, ‘vertical’ et cetera, those terms refer to when the apparatus is in a typical in-use position and/or with reference to particular orientations shown in the Figures, and are used to show and/or describe relative directions or orientations.

1. A patient interface for protecting a surgical cavity, the patient interface comprising:
one or more enclosing or outer layers defining at least in part a gas flow path capable of directing gases to the surgical site, the gas flow path having a first end and an opposed second end,
an inlet positioned at the first end, and
an outlet positioned at the second end.

2. A patient interface for protecting a surgical cavity, the patient interface comprising:
an inlet fluidly connectable with a gas source, and
an outlet,
wherein the patient interface is configured to deliver a conditioned flow of gas to the surgical site, the conditioned flow of gas
i) having a reduced outlet velocity relative to the velocity of the inlet gas,
ii) having a diffuse flow,
iii) being heated,
iv) being substantially laminar or non-turbulent, or
v) any combination of (i) to (iv).

3. (canceled)

4. A patient interface for protecting a surgical cavity, comprising
an inlet,
an outlet,
one or more enclosing or outer layers at least partly defining a gas flow path between the inlet and the outlet, the gas flow path including a formable section, and
at least one deformable element positioned within the formable section, the at least one deformable element that forms at least one bending direction for the formable section.

5. (canceled)

6. The patient interface of claim 4 wherein the deformable element is malleable.

7. (canceled)

8. (canceled)

9. The patient interface of claim 4 further comprising a gas permeable substrate.

10. (canceled)

11. (canceled)

12. The patient interface of claim 9 wherein the gas permeable substrate is, or comprises, an open cell foam.

13. (canceled)

14. (canceled)

15. (canceled)

16. The patient interface of claim 9 wherein a length and/or a width of the gas permeable substrate is greater than a height of the gas permeable substrate.

17. (canceled)

18. (canceled)

19. (canceled)

20. (canceled)

21. (canceled)

22. The patient interface of claim 4 wherein the gases exiting the outlet are substantially laminar and/or non-turbulent.

23. (canceled)

24. (canceled)

25. (canceled)

26. The patient interface of claim 4 wherein the one or more enclosing or outer layer-layers each comprise a flexible film or membrane.

27. The patient interface of claim 4 wherein the one or more enclosing or outer layers are each formed of a breathable material that allows the passage of water molecules through the material.

28. (canceled)

29. (canceled)

30. (canceled)

31. (canceled)

32. The patient interface of claim 9 further comprising a support structure at a first end, the support structure defining a housing inlet and housing outlet, the housing outlet being in fluid communication with the inlet.

33. The patient interface of claim 32 wherein the deformable element attaches to the support structure.

34. The patient interface of claim 32 wherein the support structure abuts the gas permeable substrate.

35. (canceled)

36. (canceled)

37. (canceled)

38. (canceled)

39. The patient interface of claim 32 further comprising a tube having a first and second end, the first end connectable to the housing inlet of the support structure.

40. (canceled)

41. (canceled)

42. (canceled)

43. The patient interface of claim 4 further comprising a front surface and a rear surface, and wherein the rear surface further comprises an adhesive.

44. (canceled)

45. (canceled)

46. (canceled)

47. The patient interface of claim 4 further comprising a removal tab.

48. (canceled)

49. The patient interface of claim 43 wherein the enclosing or outer layer includes a cut-out on the rear surface adjacent the outlet.

50. The patient interface of claim 9 wherein at least a portion of a side region of the gas permeable material is unobstructed.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. A method of protecting a patient from a loss of moisture and/or loss of heat and/or from an infection of a surgical site during surgery, the method comprising:
positioning an interface of on a surface in or adjacent the surgical site, the interface comprising one or more enclosing or outer layers defining at least in part a gas flow path capable of directing gases to the surgical site, the gas flow path having a first end and an opposed second end; an inlet positioned at the first end, and an outlet positioned at the second end.

63. (canceled)

64. (canceled)

65. (canceled)

66. (canceled)
